(19) United States
(12) Patent Application Publication
(10) Pub. No.: US 2023/0210845 A1
(43) Pub. Date: Jul. 6, 2023

(54) DOSING METHODS FOR TREATMENT OF CARDIOVASCULAR CONDITIONS

(71) Applicant: IMBRIA PHARMACEUTICALS, INC., Boston, MA (US)

(72) Inventors: Jaikrishna Patel, Cary, NC (US); Paul Chamberlin, Brookline, MA (US); David-Alexandre Gros, Rancho Santa Fe, CA (US); Larry Tremaine, Merritt Island, FL (US)

(21) Appl. No.: 17/922,890

(22) PCT Filed: May 3, 2021
(86) PCT No.: PCT/US2021/030450

§ 371 Date: Nov. 2, 2022

Related U.S. Application Data
(60) Provisional application No. 63/019,545, filed on May 4, 2020.

(51) Int. Cl. A61K 31/496; A61K 9/00; A61P 9/10

(52) U.S. Cl. CPC A61K 31/496; A61K 9/0053; A61P 9/10

(57) ABSTRACT

The invention provides methods of treating cardiovascular conditions and methods of increasing the efficiency of cardiac metabolism.

### CROSS-REFERENCE TO RELATED APPLICATIONS

This application claims the benefit of, and priority to, U.S. Provisional Pat. Application No. 63/019,545, filed May 4, 2021, the contents of which are incorporated by reference.

### FIELD OF THE INVENTION

The invention relates to methods of treating cardiovascular conditions and methods of increasing the efficiency of cardiac metabolism.

### BACKGROUND

Heart disease is the leading cause of death worldwide, accounting for 15 million deaths across the globe in 2015. In coronary artery disease (CAD), the most common cardiovascular disease, blood flow to the heart muscle is reduced due to accumulation of plaque in the arteries of the heart. CAD is often associated with angina, a sensation of pain or pressure in the chest. In patients with refractory angina, the pain or discomfort continues despite optimal medical therapy.

Existing therapies for treating cardiovascular disease are problematic. Several approaches that focus on restoring blood flow to the heart require risky surgical interventions. For example, coronary artery bypass graft is a major surgery associated with various complications. On the other hand, less invasive procedures, such as balloon angioplasty and stent insertion, can lead to vascular damage, blood clotting, and infection, and they are susceptible to restenosis. Many classes of drugs, such as cholesterol-lowering medicine, beta blockers, and calcium channel blockers, are also used to treat CAD, but such drugs fail to rectify changes in cardiac energy metabolism. In patients with CAD, cardiac mitochondria rely predominantly on oxidation of fatty acids rather than glucose to provide energy. Because oxidation of fatty acids is a less efficient means of producing energy, the pumping capacity of the heart is reduced. Therefore, drugs that do not restore glucose oxidation in the heart have limited efficacy. Consequently, no safe, effective therapy is available for millions of patients with CAD or refractory angina.

### SUMMARY

The invention provides dosing methods for administering compositions containing a compound that improves cardiac mitochondrial function to treat cardiac conditions. The dosing methods include providing such compositions at least once per day to patients. The compositions contain a compound that is metabolized in the body into multiple products that improve cardiac mitochondrial metabolism by independent but cooperative mechanisms. One set of metabolic products, which may include trimetazidine and its derivatives, shifts cardiac metabolism from fatty acid oxidation to glucose oxidation. Metabolic products in another set serve as precursors for synthesis of nicotinamide adenine dinucleotide (NAD⁺) and thus facilitate mitochondrial respiration. The compositions may be delivered orally, obviating the need for specialized equipment or personnel. The methods are useful for treating a wide variety of cardiovascular conditions, including CAD and refractory angina.

In an aspect, the invention provides methods of treating a cardiovascular condition in a subject by providing to a subject having, or at risk of developing, a cardiovascular condition at least one dose per day of a composition containing a compound represented by formula (VII) or (VIII):

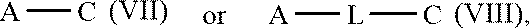

in which A is a compound that shifts cardiac metabolism from fatty acid oxidation to glucose oxidation, L is a linker, and C is a NAD⁺ precursor molecule. A may be covalently linked to C or to L, and L may be covalently linked to C.

The compound that shifts cardiac metabolism from fatty acid oxidation to glucose oxidation may be trimetazidine, etomoxir, perhexiline, a PPAR agonist, a malonyl CoA decarboxylase inhibitor, or dichloroacetate.

The NAD⁺ precursor molecule may be nicotinic acid, nicotinamide, or nicotinamide riboside.

The compound of formula (VII) or (VIII) may be PEGylated with an ethylene glycol moiety. The ethylene glycol moiety may be attached to one or more of A, L, and C. L may be or include an ethylene glycol moiety. The compound may have multiple ethylene glycol moieties, such as one, two three, four, five, or more ethylene glycol moieties. The ethylene glycol moiety may be represented by (CH₂CH₂O)ₓ, in which x = 1-15. The ethylene glycol moiety may form a covalent linkage between the compound that shifts cardiac metabolism from fatty acid oxidation to glucose oxidation and the NAD⁺ precursor molecule. The ethylene glycol moiety may be separate from a covalent linkage between the compound that shifts cardiac metabolism from fatty acid oxidation to glucose oxidation and the NAD⁺ precursor molecule. The compound that shifts cardiac metabolism from fatty acid oxidation to glucose oxidation may be a PEGylated form of trimetazidine.

The compound of formula (VII) or the compound of formula (VIII) may include nicotinic acid that is covalently linked to a PEGylated form of trimetazidine. The nicotinic acid may be covalently linked via the PEGylated moiety, i.e., via an ethylene glycol linkage. The nicotinic acid may be covalently linked via the trimetazidine moiety.

The compound of formula (VII) or the compound of formula (VIII) may have a structure represented by formula (X):

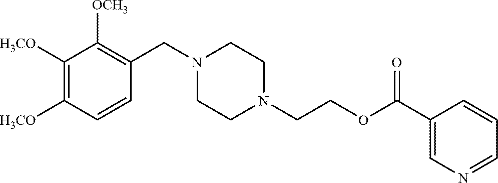 [SMILES: COc1ccc(CN2CCN(CCOC(=O)c3cccnc3)CC2)c(OC)c1OC]

The dose may be provided by any suitable route or mode of administration. The dose may be provided orally, intravenously, enterally, parenterally, dermally, buccally, topically, transdermally, by injection, subcutaneously, nasally, pulmonarily, or with or on an implantable medical device (e.g., stent or drug-eluting stent or balloon equivalents).

The composition may be provided in one dose per day. The composition may be provided in multiple doses per day. The composition may be provided in two, three, four, five, six, eight, or more doses per day.

The dose may contain from about 10 mg to about 2000 mg, from about 10 mg to about 1000 mg, from about 10 mg to about 800 mg, from about 10 mg to about 600 mg, from about 10 mg to about 400 mg, from about 10 mg to about 300 mg, from about 10 mg to about 200 mg, from about 25 mg to about 2000 mg, from about 25 mg to about 1000 mg, from about 25 mg to about 800 mg, from about 25 mg to about 600 mg, from about 25 mg to about 400 mg, from about 25 mg to about 300 mg, about 25 mg to about 200 mg, from about 50 mg to about 2000 mg, from about 50 mg to about 1000 mg, from about 50 mg to about 800 mg, from about 50 mg to about 600 mg, from about 50 mg to about 400 mg, from about 50 mg to about 300 mg, about 50 mg to about 200 mg, from about 100 mg to about 2000 mg, from about 100 mg to about 1000 mg, from about 100 mg to about 800 mg, from about 100 mg to about 600 mg, from about 100 mg to about 400 mg, from about 100 mg to about 300 mg, about 100 mg to about 200 mg, from about 200 mg to about 2000 mg, from about 200 mg to about 1000 mg, from about 200 mg to about 800 mg, from about 200 mg to about 600 mg, from about 200 mg to about 400 mg, from about 200 mg to about 300 mg, from about 300 mg to about 2000 mg, from about 300 mg to about 1000 mg, from about 300 mg to about 800 mg, from about 300 mg to about 600 mg, or from about 300 mg to about 400 mg of the compound. The dose may contain about 10 mg, about 25 mg, about 50 mg, about 100 mg, about 200 mg, about 300 mg, or about 400 mg of the compound.

The dose or doses may be provided for a defined period. One or more doses may be provided daily for at least one week, at least two weeks, at least three weeks, at least four weeks, at least six weeks, at least eight weeks, at least ten weeks, at least twelve weeks or more.

The cardiovascular condition may be an aneurysm, angina, atherosclerosis, cardiomyopathy, cerebral vascular disease, congenital heart disease, coronary artery disease, coronary heart disease, diabetic cardiomyopathy, heart attack, heart disease, heart failure, hypertension, ischemic heart disease, pericardial disease, peripheral arterial disease, rheumatic heart disease, stroke, transient ischemic attacks, or valvular heart disease. The angina may be refractory to other medical interventions.

In another aspect, the invention provides methods of improving cardiac mitochondrial function in a subject by providing to a subject at least one dose per day of a composition containing a compound represented by formula (VII) or (VIII), as defined above.

The compound of formula (VII) or the compound of formula (VIII) may have a structure represented by formula (X).

The dose may be provided by any suitable route or mode of administration, such as any of those described above. For example, the dose may be provided orally.

The composition may be provided in one dose per day. The composition may be provided in multiple doses per day. The composition may be provided in two, three, four, five, six, eight, or more doses per day.

The dose may contain the compound in a defined mass amount or range of mass amounts, such as any of those described above.

The dose or doses may be provided for a defined period, such as any of those described above.

The subject may have a condition or may be at risk of developing a condition. The condition may be a cardiovascular condition, such as any of those described above.

### BRIEF DESCRIPTION OF THE DRAWINGS

FIG. 1 shows a schematic of the study design for testing the safety and efficacy of IMB-1018972.

FIG. 2 is a table of the disposition of subjects of an FIH study of IMB-1018972.

FIG. 3 is a Schedule of Assessments for SAD part Group A5 of an FIH study of IMB-1018972.

FIG. 4 is a table of assessments given for the SAD part (and integrated FE arm) Groups A1 to A4 of an FIH study of IMB-1018972.

FIG. 5 is a table of assessments given for the MAD part of an FIH study of IMB-1018972.

FIG. 6 is a table of analysis data sets for the SAD Part (and integrated FE Arm) per dose level and total for IMB-1018972 of an FIH study of IMB-1018972.

FIG. 7 is a table of analysis data sets for the MAD Part per dose level and total for IMB-1018972 of an FIH study of IMB-1018972.

FIG. 8 is a table of a summary of demographic characteristics - SAD Part (and Integrated FE Arm) (Safety Set of an FIH study of IMB-1018972.

FIG. 9 is a table of a summary of demographic characteristics - MAD Part (Safety Set) of an FIH study of IMB-1018972.

FIG. 10 is a table of the Extent of Exposure - SAD Part (and Integrated FE Arm) (Safety Set) of an FIH study of IMB-1018972.

FIG. 11 is a table of the Extent of Exposure - MAD Part of an FIH study of IMB-1018972.

FIG. 12 is a graph of Geometric Mean IMB-1028814 Plasma Concentration-Time Profiles (Linear) - SAD Part (PK Set) of an FIH study of IMB-1018972.

FIG. 13 is a graph of Geometric Mean IMB-1028814 Plasma Concentration-Time Profiles (Semi-Logarithmic) - SAD Part (PK Set) of an FIH study of IMB-1018972.

FIG. 14 is a graph of Geometric Mean Trimetazidine Plasma Concentration-Time Profiles (Linear) - SAD Part (PK Set) of an FIH study of IMB-1018972.

FIG. 15 is a graph of Geometric Mean Trimetazidine Plasma Concentration-Time Profiles (Semi-Logarithmic) - SAD Part (PK Set) of an FIH study of IMB-1018972.

FIG. 16 is a graph of Geometric Mean IMB-1028814 + Trimetazidine Plasma Concentration-Time Profiles (Semi-Logarithmic) - SAD Part (PK Set) of an FIH study of IMB-1018972.

FIG. 17 is a graph of Geometric Mean IMB-1028814 + Trimetazidine Plasma Concentration-Time Profiles (Semi-Logarithmic) - SAD Part (PK Set) of an FIH study of IMB-1018972.

FIG. 18 is a table of Summary Statistics (Geometric Mean [Range]) of IMB-1028814, Trimetazidine, and IMB-1028814 + Trimetazidine Plasma Pharmacokinetic Parameters - SAD Part (PK Set) of an FIH study of IMB-1018972.

FIG. 19 is a table of Exploratory Analysis of Dose Proportionality for IMB-1028814 and Trimetazidine over the Dose Range of 50 mg to 400 mg IMB-1018972 under Faster Conditions -SAD Part (PK Set) of an FIH study of IMB-1018972.

FIG. 20 is a graph of Plot of Combined Individual and Geometric Mean Dose-Normalized IMB-1028814 Cₘₐₓ over the Dose Range of 50 mg to 400 mg IMB-1018972 under Fasted Conditions - SAD Part (PK Set) of an FIH study of IMB-1018972.

FIG. 21 is a graph of Plot of Combined Individual and Geometric Mean Dose-Normalized IMB-1028814 AUC₀₋ₜ over the Dose Range of 50 mg to 400 mg IMB-1018972 under Fasted Conditions - SAD Part (PK Set) of an FIH study of IMB-1018972.

FIG. 22 is a graph of Plot of Combined Individual and Geometric Mean Dose-Normalized IMB-1028814 AUC_{0-inf} over the Dose Range of 50 mg to 400 mg IMB-1018972 under Fasted Conditions - SAD Part (PK Set) of an FIH study of IMB-1018972.

FIG. 23 is a graph of Plot of Combined Individual and Geometric Mean Dose-Normalized Trimetazidine Cₘₐₓ over the Dose Range of 50 mg to 400 mg IMB-1018972 under Fasted Conditions - SAD Part (PK Set) of an FIH study of IMB-1018972.

FIG. 24 is a graph of Plot of Combined Individual and Geometric Mean Dose-Normalized Trimetazidine AUC₀₋ₜ over the Dose Range of 50 mg to 400 mg IMB-1018972 under Fasted Conditions - SAD Part (PK Set) of an FIH study of IMB-1018972.

FIG. 25 is a graph of Plot of Combined Individual and Geometric Mean Dose-Normalized Trimetazidine AUC_{0-inf} over the Dose Range of 50 mg to 400 mg IMB-1018972 under Fasted Conditions - SAD Part (PK Set) of an FIH study of IMB-1018972.

FIG. 26 is a graph of Geometric Mean IMB-1028814 Plasma Concentration-Time Profiles (Linear) - FE Arm of SAD Part (PK Set) of an FIH study of IMB-1018972.

FIG. 27 is a graph of Geometric Mean IMB-1028814 Plasma Concentration-Time Profiles (Semi-Logarithmic Scale) - FE Arm of SAD Part (PK Set) of an FIH study of IMB-1018972.

FIG. 28 is a graph of Geometric Mean Trimetazidine Plasma Concentration-Time Profiles (Linear) - FE Arm of SAD Part (PK Set) of an FIH study of IMB-1018972.

FIG. 29 is a graph of Geometric Mean Trimetazidine Plasma Concentration-Time Profiles (Semi-Logarithmic Scale) - FE Arm of SAD Part (PK Set) of an FIH study of IMB-1018972.

FIG. 30 is a graph of Geometric Mean IMB-1028814 + Trimetazidine Plasma Concentration-Time Profiles (Linear) - FE Arm of SAD Part (PK Set) of an FIH study of IMB-1018972.

FIG. 31 is a graph of Geometric Mean IMB-1028814 + Trimetazidine Plasma Concentration-Time Profiles (Semi-Logarithmic Scale) - FE Arm of SAD Part (PK Set) of an FIH study of IMB-1018972.

FIG. 32 is a table of Summary Statistics (Geometric Mean [Range]) of IMB-1028814, Trimetazidine, and IMB-128814 + Trimetazidine, and IMB-1028814 + Trimetazidine Plasma Pharmacokinetic Parameters - FE Arm of SAD Part (PK Set) of an FIH study of IMB-1018972.

FIG. 33 is a table of Exploratory Analysis of Food Effect for IMB-1028814 and Trimetazidine following Administration of 150 mg IMB-1018972 - FE Arm of SAD Part (PK Set) of an FIH study of IMB-1018972.

FIG. 34 is a table of Summary Statistics (Arithmetic Mean [SD]) of Urine Pharmacokinetic Parameters for IMB-1028814, Trimetazidine, and IMB-1028814 + Trimetazidine - SAD Part (PK Set) of an FIH study of IMB-1018972.

FIG. 35 is a graph of Geometric Mean IMB-1028814 Plasma Concentration-Time Profiles from Day 1 through Day 14 (Linear) - MAD Part (PK Set) of an FIH study of IMB-1018972.

FIG. 36 is a graph of Geometric Mean IMB-1028814 Plasma Concentration-Time Profiles from Day 1 through Day 14 (Semi-Logarithmic Scale) - MAD Part (PK Set) of an FIH study of IMB-1018972.

FIG. 37 is a graph of Geometric Mean Trimetazidine Plasma Concentration-Time Profiles from Day 1 through Day 14 (Linear) - MAD Part (PK Set) of an FIH study of IMB-1018972.

FIG. 38 is a graph of Geometric Mean Trimetazidine Plasma Concentration-Time Profiles from Day 1 through Day 14 (Semi-Logarithmic Scale) - MAD Part (PK Set) of an FIH study of IMB-1018972.

FIG. 39 is a graph of Geometric Mean IMB-1028814 + Trimetazidine Plasma Concentration-Time Profiles from Day 1 through Day 14 (Linear) - MAD Part (PK Set) of an FIH study of IMB-1018972.

FIG. 40 is a graph of Geometric Mean IMB-1028814 + Trimetazidine Plasma Concentration-Time Profiles from Day 1 through Day 14 (Semi-Logarithmic Scale) - MAD Part (PK Set) of an FIH study of IMB-1018972.

FIG. 41 is a graph of Geometric Mean IMB-1028814 Plasma Concentration-Time Profiles from Day 1 through Day 14 (Linear) - MAD Part (PK Set) of an FIH study of IMB-1018972.

FIG. 42 is a graph of Geometric Mean IMB-1028814 Plasma Concentration-Time Profiles from Day 1 through Day 14 (Semi-Logarithmic Scale) - MAD Part (PK Set) of an FIH study of IMB-1018972.

FIG. 43 is a graph of Geometric Mean Trimetazidine Plasma Concentration-Time Profiles after Dosing on Day 1 through Day 14 (Linear) - MAD Part (PK Set) of an FIH study of IMB-1018972.

FIG. 44 is a graph of Geometric Mean Trimetazidine Plasma Concentration-Time Profiles after Dosing on Day 1 through Day 14 (Semi-Logarithmic Scale) - MAD Part (PK Set) of an FIH study of IMB-1018972.

FIG. 45 is a graph of Geometric Mean IMB-1028814 + Trimetazidine Plasma Concentration-Time Profiles after Dosing on Day 1 through Day 14 (Linear) - MAD Part (PK Set) of an FIH study of IMB-1018972.

FIG. 46 is a graph of Geometric Mean IMB-1028814 + Trimetazidine Plasma Concentration-Time Profiles after Dosing on Day 1 through Day 14 (Semi-Logarithmic Scale) -MAD Part (PK Set) of an FIH study of IMB-1018972.

FIG. 47 is a table of Summary Statistics (Geometric Mean [Range]) of IMB-1028814, Trimetazidine, and IMB-1028814 + Trimetazidine Plasma Pharmacokinetic Parameters - MAD Part (PK Set) of an FIH study of IMB-1018972.

FIG. 48A and FIG. 48B is a table Summary of All TEAEs by System Organ Class, Preferred Term and Treatment - SAD Part (and integrated FE Arm) (Safety Set) with the following notifications: of an FIH study of IMB-1018972.

FIG. 49A and FIG. 49B is a table Summary of All TEAEs by System Organ Class, Preferred Term and Treatment - MAD Part (Safety Set) of an FIH study of IMB-1018972.

FIG. 50 is a table Summary of All TEAEs by Treatment, Relationship, and Severity-SAD Part (and Integrated FE Arm) (Safety Set) of an FIH study of IMB-1018972.

FIG. 51 is a table Summary of All TEAEs by Treatment, Relationship, and Severity -MAD Part (Safety Set) of an FIH study of IMB-1018972.

### DETAILED DESCRIPTION

The invention provides dosing methods for administering compositions containing a compound that improves cardiac mitochondrial function to treat cardiac conditions. In some embodiments, the methods include providing such a composition to subject one or more times per day. Because the compositions may be formulated for oral administration, the methods are simple and may be performed by a patient without direct medical supervision. The methods may be used to treat cardiovascular conditions, such as coronary artery disease (CAD) and refractory angina.

In many types of heart disease, the overall efficiency of energy production by cardiac mitochondria is diminished. In part, this is due to an increased reliance on fatty acid oxidation over glucose oxidation in many types of heart disease. Glucose oxidation is a more efficient pathway for energy production, as measured by the number of ATP molecules produced per O₂ molecule consumed, than is fatty acid oxidation. However, other metabolic changes contribute to decreased cardiac efficiency in patients with heart disease. For example, overall mitochondrial oxidative metabolism can be impaired in heart failure, and energy production is decreased in ischemic heart disease due to a limited supply of oxygen.

Glucose oxidation and fatty acid oxidation are energy-producing metabolic pathways that compete with each other for substrates. In glucose oxidation, glucose is broken down to pyruvate via glycolysis in the cytosol of the cell. Pyruvate then enters the mitochondria, where it is converted to acetyl coenzyme A (acetyl-CoA). In beta-oxidation of fatty acids, which occurs in the mitochondria, two-carbon units from long-chain fatty acids are sequentially converted to acetyl-CoA.

The remaining steps in energy production from glucose oxidation of glucose and fatty acid oxidation are common to the two pathways. Acetyl-CoA is oxidized to carbon dioxide (CO₂) via the citric acid cycle, which results in the conversion of nicotinamide adenine dinucleotide (NAD⁺) to its reduced form, NADH. NADH, in turn, drives the mitochondrial electron transport chain. The electron transport chain comprises a series of four mitochondrial membrane-bound complexes that transfer electrons via redox reactions. In doing so, the complexes pump protons across the membrane to create a proton gradient. The redox reactions of the electron transport chain require molecular oxygen (O₂). In the final step of mitochondrial energy production, the proton gradient enables another membrane-bound enzymatic complex to form high-energy ATP molecules, which are the source of energy for most cellular reactions.

The methods of the invention improve cardiac efficiency by using multiple mechanisms to alter mitochondrial metabolism. In certain embodiments, the methods entail providing compounds that are metabolized in the body into multiple products that have different effects. A first metabolic product or set of metabolic products shifts cardiac metabolism from fatty acid oxidation to glucose oxidation, and a second product or set of products promotes mitochondrial respiration. Thus, administering such compounds triggers a change in the pathway used to produce energy and concomitantly improves overall mitochondrial oxidative function. Consequently, the methods of the invention are more effective at restoring cardiac capacity in patients with heart disease than are other methods that target a single metabolic deficiency. Moreover, such methods avoid the use of risky surgical procedures that can lead to serious complications.

### Dosing Methods

In certain embodiments, methods of the invention include providing a composition containing a compound that improves cardiac mitochondrial function to a subject.

The dose may be provided by any suitable route or mode of administration. The dose may be provided orally, intravenously, enterally, parenterally, dermally, buccally, topically, transdermally, by injection, subcutaneously, nasally, pulmonarily, or with or on an implantable medical device (e.g., stent or drug-eluting stent or balloon equivalents).

Doses may be provided at any suitable interval. For example and without limitation, doses may be provided once per day, twice per day, three times per day, four times per day, five times per day, six times per day, eight times per day, once every 48 hours, once every 36 hours, once every 24 hours, once every 12 hours, once every 8 hours, once every 6 hours, once every 4 hours, once every 3 hours, once every two days, once every three days, once every four days, once every five days, once every week, twice per week, three times per week, four times per week, or five times per week.

The dose may contain a defined amount of the compound that improves cardiac mitochondrial function. For example and without limitation, the dose may contain from about 10 mg to about 2000 mg, from about 10 mg to about 1000 mg, from about 10 mg to about 800 mg, from about 10 mg to about 600 mg, from about 10 mg to about 400 mg, from about 10 mg to about 300 mg, from about 10 mg to about 200 mg, from about 25 mg to about 2000 mg, from about 25 mg to about 1000 mg, from about 25 mg to about 800 mg, from about 25 mg to about 600 mg, from about 25 mg to about 400 mg, from about 25 mg to about 300 mg, about 25 mg to about 200 mg, from about 50 mg to about 2000 mg, from about 50 mg to about 1000 mg, from about 50 mg to about 800 mg, from about 50 mg to about 600 mg, from about 50 mg to about 400 mg, from about 50 mg to about 300 mg, about 50 mg to about 200 mg, from about 100 mg to about 2000 mg, from about 100 mg to about 1000 mg, from about 100 mg to about 800 mg, from about 100 mg to about 600 mg, from about 100 mg to about 400 mg, from about 100 mg to about 300 mg, about 100 mg to about 200 mg, from about 200 mg to about 2000 mg, from about 200 mg to about 1000 mg, from about 200 mg to about 800 mg, from about 200 mg to about 600 mg, from about 200 mg to about 400 mg, from about 200 mg to about 300 mg, from about 300 mg to about 2000 mg, from about 300 mg to about 1000 mg, from about 300 mg to about 800 mg, from about 300 mg to about 600 mg, or from about 300 mg to about 400 mg of the compound. The dose may contain about 10 mg, about 25 mg, about 50 mg, about 100 mg, about 200 mg, about 300 mg, or about 400 mg of the compound.

The dose may be provided in a single dosage, i.e., the dose may be provided as a single tablet, capsule, pill, etc. Alternatively, the dose may be provided in a divided dosage, i.e., the dose may be provided as multiple tablets, capsules, pills, etc.

The dosing may continue for a defined period. For example and without limitation, doses may be provided for at least one week, at least two weeks, at least three weeks, at least four weeks, at least six weeks, at least eight weeks, at least ten weeks, at least twelve weeks or more.

The subject may be a human. The subject may be a human that has a cardiovascular condition, such as one of those described below. The subject may be a human that is at risk of developing a cardiovascular condition, such as one of those described above. A subject may be at risk of developing a condition if the subject does not meet established criteria for diagnosis of the condition but has one or more symptoms, markers, or other factors that indicate the subject is likely to meet the diagnostic criteria for the condition in the future. The subject may be a pediatric, a newborn, a neonate, an infant, a child, an adolescent, a pre-teen, a teenager, an adult, or an elderly subject. The subject may be in critical care, intensive care, neonatal intensive care, pediatric intensive care, coronary care, cardiothoracic care, surgical intensive care, medical intensive care, long-term intensive care, an operating room, an ambulance, a field hospital, or an out-of-hospital field setting.

### Compounds

Certain embodiments of the invention include providing to a subject a composition containing a compound represented by formula (VII) or (VIII):

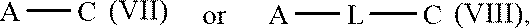

in which A is a compound that shifts cardiac metabolism from fatty acid oxidation to glucose oxidation, L is a linker, and C is a NAD⁺ precursor. Examples of each component are described in detail below. A may be covalently linked to C or to L, and L may be covalently linked to C.

The compound of formula (VII) may include nicotinic acid that is covalently linked to a PEGylated form of trimetazidine. The nicotinic acid may be covalently linked via a PEGylated moiety, i.e., via an ethylene glycol linkage. The nicotinic acid may be covalently linked via the trimetazidine moiety.

The compound of formula (VII) or the compound of formula (VIII) may have a structure represented by formula (X):

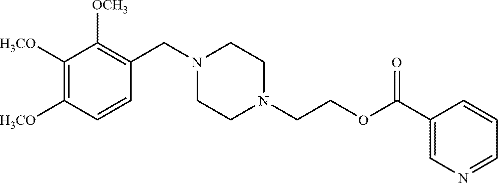 [SMILES: COc1ccc(CN2CCN(CCOC(=O)c3cccnc3)CC2)c(OC)c1OC]

Compounds of formulas (VII), (VIII), and (X) are described in, for example, International Patent Publication No. WO 2018/236745, the contents of which are incorporated herein by reference.

### Compounds That Shift Cardiac Metabolism From Fatty Acid Oxidation to Glucose Oxidation

Component A may be any suitable compound that shifts cardiac metabolism from fatty acid oxidation to glucose oxidation. Such compounds can be classified based on their mechanism of action. See Fillmore, N., et al., Mitochondrial fatty acid oxidation alterations in heart failure, ischemic heart disease and diabetic cardiomyopathy, Brit. J. Pharmacol. 171:2080-2090 (2014), the contents of which are incorporated herein by reference.

One class of glucose-shifting compounds includes compounds that inhibit fatty acid oxidation directly. Compounds in this class include inhibitors of malonyl CoA decarboxylase (MCD), carnitine palmitoyl transferase 1 (CPT-1), or mitochondrial fatty acid oxidation. Mitochondrial fatty acid oxidation inhibitors include trimetazidine and other compounds described in International Patent Publication No. WO 2002/064576, the contents of which are incorporated herein by reference. Trimetazidine binds to distinct sites on the inner and outer mitochondrial membranes and affects both ion permeability and metabolic function of mitochondria. Morin, D., et al., Evidence for the existence of [³H]-trimetazidine binding sites involved in the regulation of the mitochondrial permeability transition pore, Brit. J. Pharmacol. 123:1385-1394 (1998), the contents of which are incorporated herein by reference. MCD inhibitors include CBM-301106, CBM-300864, CBM-301940, 5-(1,1,1,3,3,3-hexafluoro-2-hydroxypropan-2-yl)-4,5-dihydroisoxazole-3-carboxamides, methyl 5-(N-(4-(1,1,1,3,3,3-hexafluoro-2-hydroxypropan-2-yl)phenyl)morpholine-4-carboxamido)pentanoate, and other compounds described in Chung, J.F., et al., Discovery of Potent and Orally Available Malonyl-CoA Decarboxylase Inhibitors as Cardioprotective Agents, J. Med. Chem. 49:4055-4058 (2006); Cheng J.F. et al., Synthesis and structure-activity relationship of small-molecule malonyl coenzyme A decarboxylase inhibitors, J. Med. Chem. 49:1517-1525 (2006); U.S. Patent Publication No. 2004/0082564; and International Patent Publication No. WO 2002/058698, the contents of each of which are incorporated herein by reference. CPT-1 inhibitors include oxfenicine, perhexiline, etomoxir, and other compounds described in International Patent Publication Nos. WO 2015/018660; WO 2008/109991; WO 2009/015485; and WO 2009/156479; and U.S. Pat. Publication No. 2011/0212072, the contents of each of which are incorporated herein by reference.

Another class of glucose-shifting compounds includes compounds that stimulate glucose oxidation directly. Examples of such compounds are described in U.S. Pat. Publication No. 2003/0191182; International Patent Publication No. WO 2006/117686; U.S. Pat. No. 8,202,901, the contents of each of which are incorporated herein by reference.

Another class of glucose-shifting compounds includes compounds that decrease the level of circulating fatty acids that supply the heart. Examples of such compounds include agonists of PPARα and PPARγ, including fibrate drugs, such as clofibrate, gemfibrozil, ciprofibrate, bezafibrate, and fenofibrate, and thiazolidinediones, GW-9662, and other compounds described in U.S. Pat. No. 9,096,538, the contents of which are incorporated herein by reference.

### Linkers

Component L may be any suitable linker. Preferably, the linker can be cleaved in vivo to release components A and B. The linker may be an alkoxy group. The linker may be polyethylene glycol of any length. The linker may be represented by (CH₂CH₂O)ₓ, in which x = 1-15 or (CH₂CH₂O)ₓ, in which x = 1-3. Other suitable linkers include 1,3-propanediol, diazo linkers, phosphoramidite linkers, disulfide linkers, cleavable peptides, iminodiacetic acid linkers, thioether linkers, and other linkers described in Leriche, G., et al., Cleavable linkers in chemical biology, Bioorg. Med. Chem. 20:571-582 (2012); International Patent Publication No. WO 1995000165; and U.S. Pat. No. 8,461,117, the contents of each of which are incorporated herein by reference.

### NAD⁺ Precursor Molecules

Component C may be any molecule that can serve as a precursor to NAD⁺ in vivo. NAD⁺ is an important oxidizing agent that acts as a coenzyme in multiple reactions of the citric acid cycle. In these reactions, NAD⁺ is reduced to NADH. Conversely, NADH is oxidized back to NAD⁺ when it donates electrons to mitochondrial electron transport chain. In humans, NAD⁺ can be synthesized de novo from tryptophan, but not in quantities sufficient to meet metabolic demands. Consequently, NAD⁺ is also synthesized via a salvage pathway, which uses precursors that must be supplied from the diet. Among the precursors used by the salvage pathway for NAD⁺ synthesis are nicotinic acid, nicotinamide, and nicotinamide riboside. By providing a NAD⁺ precursor, such as nicotinic acid, nicotinamide, or nicotinamide riboside, the compound facilitates NAD⁺ synthesis.

The inclusion of a NAD⁺ precursor in compounds of the invention allows the compounds to stimulate energy production in cardiac mitochondria in multiple ways. Component A shifts cardiac metabolism from fatty acid oxidation to glucose oxidation, which is inherently more efficient. The NAD⁺ precursor provides an essential coenzyme that cycles between oxidized and reduced forms to promote respiration. In the oxidized form, NAD⁺ drives reactions of the citric acid cycle. In the reduced form, NADH promotes electron transport to create a proton gradient that enables ATP synthesis. Consequently, the chemical potential resulting from oxidation of acetyl CoA is efficiently converted to ATP that can be used for various cellular functions.

The NAD⁺ precursor molecule may be covalently attached to the compound in any suitable manner. For example, it may linked to A or L, and it may be attached directly or via another linker. Preferably, it is attached via a linker that can be cleaved in vivo. The NAD⁺ precursor molecule may be attached via a 1,3-propanediol linkage.

### PEGylation

The compound may be covalently attached to one or more molecules of polyethylene glycol (PEG), i.e., the compound may be PEGylated. In many instances, PEGylation of molecules reduces their immunogenicity, which prevents the molecules from being cleared from the body and allows them to remain in circulation longer. The ethylene glycol moiety may serve as a linker, as described above in relation to Component L, or it may be attached to only one component, e.g., Component A, L, or C, of the compound. The ethylene glycol moiety may be separate from a covalent linkage between the compound that shifts cardiac metabolism from fatty acid oxidation to glucose oxidation and the NAD⁺ precursor molecule.

The compound may contain a PEG polymer of any size. For example, the PEG polymer may have from 1-500 (CH₂CH₂O) units. The ethylene glycol moiety may be represented by (CH₂CH₂O)ₓ, in which x = 1-15. The PEG polymer may have any suitable geometry, such as a straight chain, branched chain, star configuration, or comb configuration. The compound may be PEGylated at any site. For example, the compound may be PEGylated on component A, component L (if present), or the NAD⁺ precursor. The compound may be PEGylated at multiple sites. For a compound PEGylated at multiple sites, the various PEG polymers may be of the same or different size and of the same or different configuration.

The compound that shifts cardiac metabolism from fatty acid oxidation to glucose oxidation may be PEGylated with an ethylene glycol moiety. The compound that shifts cardiac metabolism from fatty acid oxidation to glucose oxidation may have multiple ethylene glycol moieties, such as one, two three, four, five, or more ethylene glycol moieties. The ethylene glycol moiety may be represented by (CH₂CH₂O)ₓ, in which x = 1-15. The ethylene glycol moiety may form a covalent linkage between the compound that shifts cardiac metabolism from fatty acid oxidation to glucose oxidation and the NAD⁺ precursor molecule. The ethylene glycol moiety may be separate from a covalent linkage between the compound that shifts cardiac metabolism from fatty acid oxidation to glucose oxidation and the NAD⁺ precursor molecule.

The compound of formula (VII) may include nicotinic acid that is covalently linked to a PEGylated form of trimetazidine. The nicotinic acid may be covalently linked via a PEGylated moiety, i.e., via an ethylene glycol linkage. The nicotinic acid may be covalently linked via the trimetazidine moiety.

### Isotopically-Enriched Compounds

The compounds may include one or more atoms that are enriched for an isotope. For example, the compounds may have one or more hydrogen atoms replaced with deuterium or tritium. Isotopic substitution or enrichment may occur at carbon, sulfur, or phosphorus, or other atoms. The compounds may be isotopically substituted or enriched for a given atom at one or more positions within the compound, or the compounds may be isotopically substituted or enriched at all instances of a given atom within the compound.

### Compositions

In certain embodiments, methods of the invention include providing pharmaceutical compositions containing one or more of the compounds described above. A pharmaceutical composition containing a compound may be in a form suitable for oral use, for example, as tablets, troches, lozenges, fast-melts, aqueous or oily suspensions, dispersible powders or granules, emulsions, hard or soft capsules, syrups or elixirs. Compositions intended for oral use may be prepared according to any method known in the art for the manufacture of pharmaceutical compositions and such compositions may contain one or more agents selected from sweetening agents, flavoring agents, coloring agents and preserving agents, in order to provide pharmaceutically elegant and palatable preparations. Tablets contain the compounds in admixture with non-toxic pharmaceutically acceptable excipients which are suitable for the manufacture of tablets. These excipients may be for example, inert diluents, such as calcium carbonate, sodium carbonate, lactose, calcium phosphate or sodium phosphate; granulating and disintegrating agents, for example corn starch, or alginic acid; binding agents, for example starch, gelatin or acacia, and lubricating agents, for example magnesium stearate, stearic acid or talc. Preparation and administration of compounds is discussed in U.S. Pat. No. 6,214,841 and U.S. Pat. Publication No. 2003/0232877, the contents of each of which are incorporated by reference herein.

Formulations for oral use may also be presented as hard gelatin capsules in which the compounds are mixed with an inert solid diluent, for example calcium carbonate, calcium phosphate or kaolin, or as soft gelatin capsules in which the compounds are mixed with water or an oil medium, for example peanut oil, liquid paraffin or olive oil.

Aqueous suspensions may contain the compounds in admixture with excipients suitable for the manufacture of aqueous suspensions. Such excipients are suspending agents, for example sodium carboxymethylcellulose, methylcellulose, hydroxypropylmethylcellulose, sodium alginate, polyvinylpyrrolidone, gum tragacanth and gum acacia; dispersing or wetting agents such as a naturally occurring phosphatide, for example lecithin, or condensation products of an alkylene oxide with fatty acids, for example, polyoxyethylene stearate, or condensation products of ethylene oxide with long chain aliphatic alcohols, for example heptadecaethyleneoxycetanol, or condensation products of ethylene oxide with partial esters derived from fatty acids and a hexitol such a polyoxyethylene with partial esters derived from fatty acids and hexitol anhydrides, for example polyoxyethylene sorbitan monooleate. The aqueous suspensions may also contain one or more preservatives, for example ethyl, or n-propyl p-hydroxybenzoate, one or more coloring agents, one or more flavoring agents, and one or more sweetening agents, such as sucrose or saccharin.

Oily suspensions may be formulated by suspending the compounds in a vegetable oil, for example, arachis oil, olive oil, sesame oil or coconut oil, or in a mineral oil such as liquid paraffin. The oily suspensions may contain a thickening agent, for example beeswax, hard paraffin or cetyl alcohol. Sweetening agents such as those set forth above, and flavoring agents may be added to provide a palatable oral preparation. These compositions may be preserved by the addition of an anti-oxidant such as ascorbic acid.

Dispersible powders and granules suitable for preparation of an aqueous suspension by the addition of water provide the compounds in admixture with a dispersing or wetting agent, suspending agent and one or more preservatives. Suitable dispersing or wetting agents and suspending agents are exemplified, for example sweetening, flavoring and coloring agents, may also be present.

The pharmaceutical compositions use in methods of the invention may also be in the form of oil-in-water emulsions. The oily phase may be a vegetable oil, for example olive oil or arachis oil, or a mineral oil, for example liquid paraffin or mixtures of these. Suitable emulsifying agents may be naturally-occurring gums, for example gum acacia or gum tragacanth, naturally occurring phosphatides, for example soya bean, lecithin, and esters or partial esters derived from fatty acids and hexitol anhydrides, for example sorbitan monooleate and condensation products of the said partial esters with ethylene oxide, for example polyoxyethylene sorbitan monooleate. The emulsions may also contain sweetening and flavoring agents.

Syrups and elixirs may be formulated with sweetening agents, such as glycerol, propylene glycol, sorbitol or sucrose. Such formulations may also contain a demulcent, a preservative, and agents for flavoring and/or coloring. The pharmaceutical compositions may be in the form of a sterile injectable aqueous or oleaginous suspension. This suspension may be formulated according to the known art using those suitable dispersing or wetting agents and suspending agents which have been mentioned above. The sterile injectable preparation may also be in a sterile injectable solution or suspension in a non-toxic parenterally acceptable diluent or solvent, for example as a solution in 1,3-butanediol. Among the acceptable vehicles and solvents that may be employed are water, Ringer’s solution and isotonic sodium chloride solution. In addition, sterile, fixed oils are conventionally employed as a solvent or suspending medium. For this purpose any bland fixed oil may be employed including synthetic mono- or di-glycerides. In addition, fatty acids such as oleic acid find use in the preparation of injectables.

### Cardiac Conditions

The methods of the invention may be used to treat a cardiac condition in a subject. For example and without limitation, the cardiac condition may be aneurysm, angina, atherosclerosis, cardiomyopathy, cerebral vascular disease, congenital heart disease. coronary artery disease (CAD), coronary heart disease, diabetic cardiomyopathy, heart attack, heart disease, heart failure, high blood pressure (hypertension), ischemic heart disease, pericardial disease, peripheral arterial disease, refractory angina, rheumatic heart disease, stable angina, stroke, transient ischemic attack, unstable angina, or valvular heart disease.

Angina pectoris (angina) is chest pain or pressure that is typically due to insufficient blood flow to the heart muscle. The pain or discomfort is retrosternal or left-sided and may radiate to the left arm, neck, jaw, or back. Several classifications of angina are known.

Stable angina, also called effort angina, is related to myocardial ischemia. In stable angina, chest discomfort and associated symptoms are usually triggered by some physical activity, such as running or walking, but symptoms are minimal or non-existent when the patient is at rest or has taken sublingual nitroglycerin. Symptoms typically abate several minutes after activity and recur when activity resumes. Symptoms may also be induced by cold weather, heavy meals, and emotional stress.

Unstable angina is angina that changes or worsens. Unstable angina has at least one of the following features: (1) it occurs at rest or with minimal exertion, usually lasting more than 10 minutes, (2) it is severe and of new onset, i.e., within the prior 4-6 weeks, and (3) it occurs with a crescendo pattern, i.e., distinctly more severe, prolonged, or frequent than before.

Cardiac syndrome X, also called microvascular angina, is angina-like chest pain, in the context of normal epicardial coronary arteries on angiography. Its primary cause is unknown, but factors apparently involved are endothelial dysfunction and reduced flow in the tiny resistance blood vessels of the heart. Microvascular angina may be part of the pathophysiology of ischemic heart disease.

Refractory angina is a chronic condition (≥ 3 months in duration) in which angina (1) occurs in the context of coronary artery disease (CAD), (2) cannot be controlled by a combination of optimal medical therapy, angioplasty, or bypass surgery, and (3) in which reversible myocardial ischemia has been clinically established to be the cause of the symptoms.

### Increasing Cardiac Efficiency

The compounds of the invention are useful for improving cardiac efficiency. A variety of definitions of cardiac efficiency exist in the medical literature. See, e.g.. Schipke, J.D. Cardiac efficiency, Basic Res. Cardiol. 89:207-40 (1994); and Gibbs, C.L. and Barclay, C.J. Cardiac efficiency, Cardiovasc. Res. 30:627-634 (1995), incorporated herein by reference. One definition of cardiac mechanical efficiency is the ratio of external cardiac power to cardiac energy expenditure by the left ventricle. See Lopaschuk G.D., et al., Myocardial Fatty Acid Metabolism in Health and Disease, Phys. Rev. 90:207-258 (2010), incorporated herein by reference. Another definition is the ratio between stroke work and oxygen consumption, which ranges from 20-25% in the normal human heart. Visser, F., Measuring cardiac efficiency: is it useful? Hear Metab. 39:3-4 (2008), incorporated herein by reference. Another definition is the ratio of the stroke volume to mean arterial blood pressure. Any suitable definition of cardiac efficiency may be used to measure the effects of compounds of the invention

### EXAMPLES

### Study Design

The efficacy of methods of the invention is analyzed in a randomized, double-blinded, parallel-group, placebo-controlled study in patients with refractory angina. A total of approximately 600 men and women, aged 18 through 85 years, are randomly assigned equally across 2 groups to receive IMB-1018972 200 mg tablets orally BID (twice per day) or matching placebo for 16 weeks. IMB-1018972 has the structure of formula (X).

Patients participate in the study for approximately 22 weeks, including:
a 4-week screening period (Day -28 to Day -1) during which screening and baseline procedures are performed
a 16-week double-blinded treatment period
a 2-week safety follow-up period: safety follow-up visit, approximately 14 days after the last dose of study drug

The primary endpoint is change from baseline up to Week 8 in TED and the primary time point is Week 8 or early termination, if prior to Week 8 (the same endpoint at the Week 16 time point is an exploratory endpoint). The primary time point for the secondary endpoints is either the Week 8 or Week 16 time point as specified below.

Patients are monitored throughout the study for safety and tolerability. Randomization is facilitated by the use of an IXRS. Study drug is packaged and managed in an appropriate manner to maintain the integrity and blind of the study.

To be eligible for screening (pre-screening), patients should meet those inclusion and exclusion requirements that can be assessed by review of medical history (eg, must have angina, on optimal therapy, not a candidate for revascularization) and do not require any intervention or procedure to make the assessment.

There are 2 visits planned during the screening period, separated by a minimum of 2 weeks. For those patients who are eligible for randomization to treatment, a third study visit is scheduled. During the screening period, procedures, screening laboratory analyses, ETTs, diaries, actigraphy assessment, and other assessments outlined in Table 1 are evaluated.

TABLE 1

| Procedure | Visit 1 | Visit 2 | Visit 3 | Visit 4 | Visit 5 | Visit 6 | Visit 7 |
|---|---|---|---|---|---|---|---|
| | Weeks -3 to 1 | | | Week 4 | Week 8 | Week 16 (End of Treatment) | Follow-up 14 days After the Last dose of Study Drug |
| Study Day | | | | 28 | 56 | 112 | 126 |
| Visit Window (Days) I | | | | -3/+7 | -3/+7 | -3/+7 | -3/+7 |
| Informed consent | X | | | | | | |
| Inclusion/exclusion | X | | X | | | | |
| Medical history | X | | | | | | |
| Demographics | X | | | | | | |
| Vital sign measurements³ | X | | X | X | X | X | X |
| Height | X | | | | | | |
| Weight | X | | | | | | X |
| Physical examination | X | | X | X | X | X | X |
| Prior/concomitant medications | X | | X | X | X | X | X |
| Clinical laboratory testing^{b} | X | | X | X | X | X | X |
| Serum pregnancy test | X | | X | | | X | X |
| Urine pregnancy test | | | X | X | | | |
| Serology (HBsAg, HCV antibody, HIV-⅟HIV-2 antibodies)^{c} | X | | | | | | |
| Urine drug screen | X | | | | | | |
| Standard rest 12-lead ECG (supine)^{d} | X | | X | X | X | X | X |
| Randomization^{e} | | | X | | | | |
| Study drug dispensing | | | X | X | X | | |
| Procedure | Visit 1 | Visit 2 | Visit 3 | Visit 4 | Visit 5 | Visit 6 | Visit 7 |
| Study Day | -28 to -14 | -14 to -7 | 0 | 28 | 56 | 112 | 126 |
| Visit Window (Days) I | | | | -3/+7 | -3/+7 | -3/+7 | -3/+7 |
| Study Day | -28 to -14 | -14 to -7 | 0 | 28 | 56 | 112 | 126 |
| Visit Window (Days) | | | | -3/+7 | -3/+7 | -3/+7 | -3/+7 |
| Study drug compliance and accountabilityf | | | | X | X | X | |
| Modified Bruce protocol treadmill ETTg | X | Xh | | | X1 | X1 | |
| Pharmacokinetic blood samplingJ | | | | Xk | Xg,k | Xg,k | |
| Blood sampling for exploratory analysis of clinical laboratory parameters and biomarkers of cardiovascular health/diseasel,j | X | | | | X | X | |
| Angina frequency and NTG use^{m} | X (daily; Visit 1 through Visit 6) | | | | | | |
| Actigraphy recording and data upload on patient’s data hubⁿ | X | X (Visit 1 through Visit 6) | | | | X | |
| Actigraphy data upload at site¹¹ | | X | X | X | X | X | |
| PGI-S^{o} | X (weekly; Visit 1 through Visit 6) | | | | | | |
| PGI-C^{o} | | | | X (weekly; Visit 4 through Visit 6) | | | |
| CCS grading of angina pectoris | X | | X | X | X | X | |
| SAQ^{p} | X | | X | X | X | X | |
| Study Day | -28 to -14 | -14 to -7 | 0 | 28 | 56 | 112 | 126 |
| Visit Window (Days) | | | | -3/+7 | -3/+7 | -3/+7 | -3/+7 |
| SF-12v2p | X | | X | X | X | X | |
| CGI-S^{P} | X | | X | X | X | X | |
| CGI-C^{p} | | | | X | X | X | |
| AE monitoring | X | | X | X | X | X | X |
| Study disposition | | | | | | | X |

^{a}Vital signs including temperature (oral, tympanic, or skin), pulse, respiratory rate, and systolic and diastolic blood pressure) will be measured in a semi-supine position after 5-minute rest and before any 12-lead ECG assessment or blood sampling is performed. Two readings of blood pressure and pulse will be taken and averaged to give the measurement to be recorded in the eCRF.
^{b}Clinical laboratory testing will include hematology, serum chemistry, coagulation, lipid profile, and urinalysis.
^{c}HCV viral load and genius HIV1/2 differentiation will be performed as confirmatory test for HCV antibody -positive patients and HIV1/2 antibody-positive patients, respectively.
^{d}Prior to each modified Bruce protocol treadmill ETT, a standard rest 12-lead ECG with electrodes in the standard position (NOT TORSO LEADS) with visible calibration pulse, and paper speed of 25 mm/sec will be obtained.
^{e}To qualify for randomization to the treatment period, the following must be met regarding the results for both screening ETTs: (a) patient must have stopped the ETT due to angina on each test, (b) TED must fall between 3 and 9 minutes on each test, (c) for those patients with interpretable ECGs, at least 0.5 mm exercise-induced ST segment depression must occur with onset <9 minutes on at least 1 of the screening ETTs to document inducible myocardial ischemia, (d) difference in TED between the 2 qualifying ETTs must not exceed 20% of the longer test or 1 minute, whichever limit is smaller.
^{f}Study drug compliance will be assessed by tablet count.
^{g}The morning dose of antianginal medications (including IMB-1018972 or placebo) will be withheld (in order to perform the ETT and PK blood collection at trough levels) and resumed after completion of the ETT. Modified Bruce protocol ETT tracings will be interpreted by Saint-Louis University core ECG laboratory.
^{h}Patients who are not eligible after the second screening ETT will be considered screening failures. However, if in the opinion of the investigator a third ETT is warranted, the investigator may consult with the study medical monitor. The study medical monitor may authorize a third ETT if warranted. A third screening ETT must occur a minimum of 5 days but no more than 10 days after the second screening ETT. The value for the baseline ETT parameter TED will be obtained from the last observation on the screening ETT closest to randomization (Visit 3).
ⁱIf the patient discontinues their assigned treatment but continues the study in order to collect data for supplemental analysis, then the patient should return to the site for an unscheduled visit to perform the ETT for the primary endpoint, as soon after discontinuation of study drug as practicable. If the patient discontinues from the study, then every effort should be made to have the patient complete an ETT at an early termination visit, which should occur as soon after withdrawal from the study as practicable.
^{j}Blood collection can be performed either in the fasted or fed state.
^{k}The time of the last dose of study drug and the time of PK blood sampling will be recorded in the eCRF.
^{l}Clinical laboratory parameters and biomarkers that reflect cardiovascular health and/or disease, including but not limited to, CRP, HbA1c, NT-proBNP, troponin, and antifibrotic biomarkers.
^{m}Angina frequency and NTG use will be reported by the patient in an electronic diary each evening for a minimum of 14 days prior to Visit 3 and each evening during the double-blinded treatment period through Visit 6.
ⁿPatients will be asked to wear an activity monitor for 24 hours a day (except as specified in the Actigraph patient guide and while uploading activity data or recharging the device) to measure their movement from Visit 1 through Visit 6. The patient should wear the activity monitor for a minimum of 15 hours per day for a day to be considered compliant. The site should monitor compliance and reeducate the patient if compliance is less than 15 hours per day. The activity monitor and a data hub will be dispensed to the patient at Visit 1. Patients will upload activity data as specified in the Actigraph patient guide. The site should upload activity data at the scheduled visits. The activity monitor and data hub will be returned to the site at Visit 6 for a final data transfer and completion of the activity assessment.
^{o}PGI-S and PGI-C will be completed weekly by the patient in an electronic diary.
^{p}The SAQ, SF-12v2, CGI-S, and CGI-C will be completed on the site electronic device.

Patients are provided a wearable activity monitor and an electronic diary at the time of the first ETT (Visit 1) and are instructed to use these devices for a minimum of 2 weeks and to return them to the site at the time of the second ETT (Visit 2). These devices are not provided to patients who clearly have not met the requirements of the first ETT (described below) or patients who are screen failures after Visit 1. To qualify for randomization to the treatment period, at least 2 screening modified Bruce protocol treadmill ETTs are performed and interpreted by a core ECG laboratory.

At screening Visit 1, informed consent is obtained from patients prior to performing any protocol procedures. Consented patients have their medical history, demographics, and concomitant medications reviewed; vital sign measurements, physical examination, height and weight measurements, standard rest 12-lead ECG, clinical laboratory tests (hematology, serum chemistry, coagulation, lipids, and urinalysis), serum pregnancy test (for women of child-bearing potential), serology tests, and urine drug screen are performed. Review of the inclusion and exclusion criteria is documented. In particular, prior antianginal medication history (eg, start dates, discontinuation dates, tolerability history) is documented. Patients also undergo their first modified Bruce protocol treadmill ETT. All antianginal medications are withheld on the morning of the ETTs and resumed after the ETT. Patients may be determined to be screen failures after the procedures and results of screening Visit 1 are reviewed.

At screening Visit 2, a second modified Bruce protocol ETT is performed a minimum of 14 days later and up to 7 days prior to randomization. This time period of 14 days allows the establishment of baseline data for actigraphy, angina frequency, and NTG use. The activity monitor and electronic diaries are returned to the site on this visit and are reissued to those patients who qualify for randomization at Visit 3.

To qualify for randomization to the treatment period, the following must be met regarding the results for both screening ETTs: (a) patient must have stopped the ETT due to angina on each test, (b) TED must fall between 3 and 9 minutes on each test, (c) for those patients with interpretable ECGs, at least 0.5 mm exercise-induced ST segment depression must occur with onset <9 minutes on at least 1 of the screening ETTs to document inducible myocardial ischemia, (d) difference in TED between the 2 qualifying ETTs must not exceed 20% of the longer test or 1 minute, whichever limit is smaller.

Patients who are not eligible after the second screening ETT are considered screening failures. However, if in the opinion of the investigator a third ETT is warranted, the investigator may consult with the study medical monitor. The study medical monitor may authorize a third ETT, if warranted. A third screening ETT must occur a minimum of 5 days but no more than 10 days after the second screening ETT.

The value for the baseline ETT parameter TED is obtained from the last observation on the screening ETT closest to randomization (Visit 3).

Prior to randomization, the investigator re-reviews all eligibility criteria. For those patients who meet the criteria, the IXRS assigns a randomization number and designate the study drug to dispense to the patient; the patient uses the designated bottle to administer the first dose of double-blinded study drug at home in the evening of Visit 3 and starts BID dosing the following day.

Study drug supply is shipped to the investigator in time for randomization.

After randomization, patients are scheduled for site visits at Weeks 4 (Visit 4), 8 (Visit 5), and 16 (Visit 6) during the double-blinded treatment period. A modified Bruce protocol ETT is performed at Weeks 8 and 16 (Visits 5 and 6); additional safety and efficacy assessments are performed at these visits. Pharmacokinetic blood samples are collected for population PK/PD at Visits 4, 5, and 6; the time of last study drug and time of PK blood collection is recorded in the eCRF; blood collection for plasma PK can be performed either in the fasted or fed state. At Visits 5 and 6, the morning dose of antianginal medications (including IMB-1018972 or placebo) is withheld (in order to perform the ETT and PK blood collection at trough levels) and resumed after completion of the ETT or PK blood collection.

A safety follow-up visit (Visit 7) is scheduled approximately 14 days after the last dose of study drug.

Angina frequency and NTG use is reported by the patient in an electronic diary each evening for a minimum of 14 days prior to the randomization visit (Visit 3), and then each evening throughout the double-blinded treatment period through Visit 6. Actigraphy data is collected during the same time periods using a wearable activity monitor. The data collected prior to the randomization visit (Visit 3) serves as the baseline data for the angina frequency, NTG use, and activity data.

The PGI-S is completed weekly by the patient in an electronic diary during the screening period and during the double-blinded treatment period (Visit 1 through Visit 6). The PGI-C is completed weekly by the patient in an electronic diary during the double-blinded treatment period (Visit 4 through Visit 6).

The SAQ, SF-12v2, CCS grading system of angina pectoris, and CGI-S is completed at baseline (Visit 1), at the randomization visit (Visit 3), and at Weeks 4, 8, and 16 (Visits 4, 5, and 6, respectively). The CGI-C is completed at Weeks 4, 8, and 16 (Visits 4, 5, and 6, respectively).

A blood collection is performed at baseline and at Weeks 8 and 16 (Visits 5 and 6) for exploratory analysis of clinical laboratory parameters and biomarkers that reflect cardiovascular health and/or disease (including, but not limited to, CRP, HbA1c, NT-proBNP, troponin, antifibrotic biomarkers); blood collection for these assessments can be performed either in the fasted or fed state.

A safety follow-up visit (Visit 7) is scheduled approximately 14 days after the last dose of study drug (either after study completion or early discontinuation from the study) to monitor for safety and query for any AEs.

The full schedule of assessments is provided in Table 1.

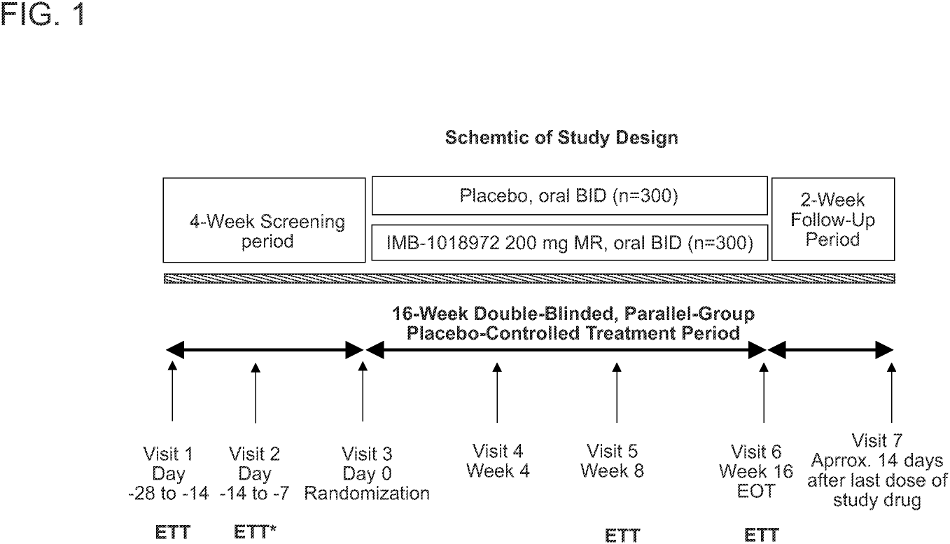

FIG. 1 shows a schematic of the study design for testing the safety and efficacy of IMB-1018972.

### Rationale for Study Design

Current Class 1 recommendations for the treatment of stable ischemic heart disease symptoms from the ACC (Fihn et al 2012) recommend medical therapy with anti-ischemic agents in conjunction with treatment of other cardiovascular risk factors (eg, hypertension, diabetes, smoking cessation) for the treatment of symptoms. Pharmacological agents aimed at improving symptoms include beta-blockers as a first line treatment. Calcium channel blockers or long-acting nitrates should be prescribed for relief of symptoms when beta-blockers are contraindicated or cause unacceptable side effects. Calcium channel blockers and long-acting nitrates should be prescribed for symptom relief if initial treatment with beta-blockers is unsuccessful. Sublingual NTG or NTG spray is recommended for immediate relief.

The traditional hemodynamic approach to reducing oxygen demand by the use of beta-blockers, calcium antagonists, and nitrates is well established. The principal mechanism of achieving a reduction in myocardial oxygen demand is by decreasing blood pressure, contractility, and heart rate. However, when titrated to effect, these agents often reach a plateau of hemodynamic suppression, where adding further dose increments or agents with a similar mechanism of action confers no benefit symptomatically, whereas adverse effects increase. This may be especially problematic in the elderly where the side effects limit their quality of life.

Many patients with angina pectoris that have persistent bouts of angina despite undergoing revascularization procedures or aggressive medical treatment with currently approved drugs, or both, are often referred to as patients with refractory angina. Alternative but complementary metabolic mechanisms for reducing ischemia that do not reduce oxygen demand or increase blood supply have been extensively investigated. By modifying the energy substrates, these metabolic agents reduce ischemia and bring about an improvement in symptoms. IMB-1018972 is one such agent and the drug that is tested in this clinical study.

The randomized, placebo-controlled, parallel-group study design of testing IMB-1018972 on top of optimal medical care in comparison to placebo plus optimal medical care is a strong randomized controlled trial design to test the hypothesis that IMB-1018972 may provide benefit to the millions of patients who experience persistent angina despite optimal therapy.

The dose selection of 200 mg tablet, given orally BID, was based on the comparison of the PK IMB-1018972 compared with the effective dose of the currently approved drug TMZ (outside of the US).

### Patient Selection

Approximately 600 patients are randomly assigned at approximately 190 sites globally, including the US and Canada. Patients are assigned to study drug only if they meet all of the inclusion criteria and none of the exclusion criteria.

Deviations from the inclusion and exclusion criteria (ie, protocol violations) are not allowed because they can potentially jeopardize the scientific integrity of the study, regulatory acceptability, or patient safety. Therefore, adherence to the eligibility criteria as specified in the protocol is essential.

### Inclusion Criteria

Each patient must meet all of the following criteria to be randomized into this study:
1. Is between the ages of 18 and 85 years, inclusive.
2. Is capable of understanding the written informed consent, and providing signed, dated, and witnessed written informed consent.
3. Has complied with and is willing to continue to comply with the specified procedures (eg, angina diary during screening) and complete specific follow-up evaluations.
4. Has coronary artery disease confirmed by at least one of the following:
   ◯ Documented prior MI, CABG surgery, or PCI -OR-
   ◯ Angiography performed within the 24 months prior to Visit 1 confirming CAD (eg, evidence of >70% stenosis of at least one major coronary artery or diffuse CAD).
5. Has evidence of stress-induced ischemia documented by either:
   ◯ Protocol-specified ETT ECG demonstrating at least 0.5 mm exercise-induced ST segment depression with onset <9 minutes on at least 1 of the screening ETT-OR-
   ◯ Prior evidence of stress-induced reversible perfusion defect in the last 24 months (without intervening revascularization) identified by at least one of the following:
      Radionuclide imaging study
      Echocardiographic imaging study
      FFR <0.8
      IFR <0.89
      FFR-CT <0.8
      CFR <2.5
6. Has a minimum 3-month history of exertional angina, including angina with a minimum frequency of 2 anginal episodes per week on average for the 2 weeks prior to enrolling in the study while on optimal guideline-directed medical therapy for their angina.
7. Is on optimal, dual agent, antianginal therapy for their angina for at least 1 month prior to the first screening ETT. Consistent with ACC/AHA and ESC Class I guideline directed medical therapy (GDMT), criteria for optimal therapy include treatment with both: (a) a beta-adrenergic blocking agent and treatment with (b) a calcium channel blocker OR a long-acting nitrate. If unacceptable side effects are intolerable and documented with a therapy listed in (a) or (b), treatment with only 1 antianginal medication is acceptable. Additionally, patients should be treated with short-acting nitrates per GDMT.
8. Is currently not clinically indicated for coronary revascularization (ie, PCI or CABG) in the opinion of the principle investigator at the time of screening through 6 months after randomization.
9. Has a CCS score of II, III or stabilized IV for 1 month prior to screening.
10. Meets the following requirements after 2 screening modified Bruce protocol ETTs:
   a. Stopping the treadmill test due to angina on each test
   b. Total exercise duration must fall between 3 and 9 minutes on each test
   c. To document inducible myocardial ischemia:
      For those patients with interpretable ECGs, at least 0.5 mm exercise- induced ST segment depression must occur with onset <9 minutes on at least 1 of the screening ETTs.
   d. Difference in TED between the 2 qualifying ETTs must not exceed 20% of the longer test or 1 minute, whichever limit is smaller.
   e. Consideration of a third ETT may be discussed by the investigator with the study medical monitor, who may authorize the performance of a third ETT a minimum of 5 days and no more than 10 days after the second ETT.

### Exclusion Criteria

Patients meeting any of the following criteria are excluded from the study:
1. If any of the following have occurred:
   In the prior 6 months:
      ◯ Diagnosis of NYHA Class 3 or 4 (heart failure) o Hospitalization for heart failure o Coronary artery bypass graft surgery
      ◯ Cardiac resynchronization therapy placement or adjustments to CRT parameters
      ◯ Implantable cardioverter defibrillator or biventricular pacemaker placement
   In the prior 3 months:
      ◯ Hospitalization for a cardiovascular indication o
      ◯ Cerebral vascular accident o
      ◯ Transient ischemic attack o
      ◯ Percutaneous coronary intervention
   In the prior 1 month:
      ◯ Use of TMZ, perhexiline, or meldonium.
2. Has a history of moderate to severe valvular heart disease defined as aortic stenosis (valve area <1.5 cm2), aortic insufficiency, mitral stenosis (valve area <1.5 cm2), or mitral valve regurgitation of grade 3 or worse.
3. Has significant hepatic disease, with increased liver function tests such as total bilirubin, aspartate aminotransferase, or alanine aminotransferase more than 2 times of ULN at baseline (excluding patients with documented history of Gilbert syndrome).
4. Has severe renal impairment (ie, creatinine clearance <30 mL/min at screening, measured using 4-variable modification of diet in renal disease equation).
5. Has a history of Parkinson disease, parkinsonian symptoms, tremors, restless leg syndrome, or other related movement disorders.
6. Has a history of vasospastic angina or microvascular angina.
7. Has an exacerbating cause for angina (eg, anemia [ie, hemoglobin <10 g/dL], uncontrolled hypertension [ie, BP >160/90 mmHg], hyperthyroid, or rapid AF [ie, AF with average rate >120 beats per minute]) at screening.
8. Has long-QT and life-threatening LV arrhythmia.
9. Has comorbidities limiting life expectancy to less than 3 years.
10. Is pregnant, planning pregnancy, or lactating.
11. Has a history of alcohol abuse or drug addiction.
12. Has a positive test for drugs (eg, opiates, methadone, cocaine, amphetamines [including ecstasy], or barbiturates) at screening.
13. Has a positive test for HBsAg, HCV antibodies, or HIV1/2 antibodies at screening.
14. Is participating in another research study.
15. Is an employee or family member of the investigator or study site personnel.

### Discontinuation From Study Treatment and/or Withdrawal From the Study

Patients may discontinue from the study drug or withdraw from the study at any time and for any reason without prejudice to their future medical care by the investigator or at the study site. Every effort should be made to keep patients in the study, including if a patient decides to prematurely discontinue study drug. The reasons for patients discontinuing the study drug and/or withdrawing from the study are recorded. A patient may be discontinued from study drug or withdrawn from the study for any of the following reasons:
1. The patient does not meet the protocol inclusion or exclusion criteria.
2. The patient is noncompliant with the protocol.
3. The patient has a serious or intolerable AE(s) that, in the investigator’s opinion, requires discontinuation from study drug or withdrawal from the study.
4. The patient has laboratory safety results that reveal clinically significant hematological or biochemical changes from the baseline values.
5. The patient has symptoms or an intercurrent illness not consistent with the protocol requirements or that justify withdrawal.
6. The patient is lost to follow-up.
7. Other reasons (eg, pregnancy, development of contraindications of use of study drug).
8. The patient withdraws consent, or the investigator or sponsor decides to discontinue their participation in the study.

### Discontinuation From Study Treatment

In rare instances, it may be necessary for a patient to permanently discontinue the study drug (definitive discontinuation). If the study drug is definitively discontinued, the patient remains in the study for continuing evaluations. Patients who discontinue their assigned treatment may continue on study in order to collect data for supplemental analysis; patients should return to the site for an unscheduled visit to perform the ETT for the primary endpoint, as soon after discontinuation of study drug as practicable. Data to be collected at the time of discontinuation of the study drug and follow-up and for any further evaluations that need to be completed are provided in Table 1.

### Withdrawal From the Study

If a patient discontinues from the study, then every effort should be made to have the patient complete an ETT at an early termination visit, which should occur as soon after withdrawal from the study as practicable. Data to be collected at the time of discontinuation from the study are provided in Table 1.

### Lost to Follow-Up

A patient is considered lost to follow-up if he or she repeatedly fails to return for scheduled visits and is unable to be contacted by the study site.

The following actions must be taken if a patient fails to return to the clinic for a required study visit:
The site must attempt to contact the patient and reschedule the missed visit as soon as possible and counsel the patient on the importance of maintaining the assigned visit schedule and ascertain whether or not the patient wishes to and/or should continue in the study.
Before a patient is deemed lost to follow-up, the investigator or designee must make every effort to regain contact with the patient (where possible, 3 telephone calls and, if necessary, a certified letter to the patient’s last known mailing address or local equivalent methods). These contact attempts should be documented in the patient’s medical record or study file.

If the patient continues to be unreachable, he/she is considered to have withdrawn from the study with a primary reason of lost to follow-up.

### Study Treatments

### Method of Assigning Patients to Treatment Groups

Patients are randomly assigned at the randomization visit (Visit 3) to receive IMB-1018972 (active drug) or placebo in a 1:1 allocation ratio. An IXRS system is used to administer the randomization schedule. The randomization schedule is generated using SAS® software Version 9.4 or higher (SAS Institute Inc, Cary, North Carolina). The IXRS links sequential patient randomization numbers and their corresponding blinded treatment assignment to study drug identifiers for dispensing of study drug. The randomization schedule is stratified by region (North America, Western Europe, Eastern Europe, and optionally, rest-of-world) and disease severity, as categorized by CCS angina grades (Grade II, and Grade III combined with stable Grade IV). Treatment is assigned by blocks within a stratum, and countries are assigned to their region stratum. Lastly, as study sites may be added during the course of the study, new countries are assigned to a region with similar patient population, and a fourth rest-of-world region may be employed.

### Treatments Administered

Study drug (IMB-1018972 or placebo) tablets are dispensed to patients at the time points specified in Table 1.

IMB-1018972 200 mg or placebo are orally administered BID for 16 weeks. Each dose consists of a total of 1 tablet consumed with food and at least 240 mL (approximately 8 oz) water.

### Identity of Study Drug

IMB-1018972 tablets consist of white-colored tablets containing 200 mg of IMB-1018972 formulation. Tablets are packaged in bottles of 36 blinded specific to the randomization sequence.

Placebo tablets are identical in appearance to the IMB-1018972 tablets but do not have the active compound. These tablets are packaged and consumed in the same manner as the active study drug.

The study site is provided with adequate supplies of IMB-1018972 and matching placebo and no expired tablets are dispensed to study patients.

### Study Drug Packaging and Storage

IMB-1018972 and matching placebo are prepared in bottles and shipped to the study site. The appropriate number of bottles are dispensed to patients to ensure sufficient quantity to cover until the next scheduled visit plus at least 8 additional days.

IMB-1018972 is stored in a secure area (eg, a locked cabinet), protected from moisture, and kept at a controlled room temperature not to exceed 15° C. - 25° C. (59° F. - 77° F.).

### Study Drug Accountability

Study drug dispensing and accountability checks are performed at the time points specified in Table 1.

The investigator maintains accurate records of receipt of all study drugs, including dates of receipt. In addition, accurate records are kept regarding when and how much study drug is dispensed and returned by each patient in the study. Only patients enrolled in the study may receive the study drug and only authorized site personnel may supply or administer the study drug. Reasons for departure from the expected dispensing regimen must also be recorded. At the completion of the study, to satisfy regulatory requirements regarding drug accountability, all study drug are reconciled and retained or destroyed according to applicable regulations.

### Overdose Management

For this study, any dose of IMB-1018972 greater than 800 mg within a 24-hour time period is considered an overdose. Any overdose must be promptly reported to PPD Pharmacovigilance. Overdose itself is not to be reported as an AE. However, any AEs associated with the overdose are to be reported on relevant AE/SAE sections in the eCRF.

In the event of an overdose, the investigator should notify the study medical monitor.

### Medication Errors

Medication errors with or without an associated AE should be recorded as medication errors in the eCRF. A mis-dosing protocol deviation (Section 11.2.2) should be reported as medication error if it was an “unintended error”.

Medication errors and associated nonserious AE should be recorded in the eCRF as described in Section 6.2.1.3. Medication errors and an associated SAE should be recorded in the eCRF and also reported to PPD Pharmacovigilance as described in Section 6.2.1.3.1.

Patients should be observed and be provided with a supportive standard of care.

### Blinding

The current study is a randomized, double-blinded, parallel-group, placebo-controlled clinical trial. Patients are randomly assigned in a 1:1 allocation ratio to receive IMB-1018972 or placebo. An IXRS is used to enter patient information (eg, qualification criteria and randomization strata) and provide study drug identifiers for dispensing of study drug to the blinded designated study staff member. All study drugs appear identical, in order to protect the study blind.

In order to keep the study blind during the study, any member of the study team involved in study conduct or participating in data handling decisions, the investigator, and patients and their families remain blinded to the patient treatment assignments until the database lock.

### Breaking the Blind

A patient’s treatment assignment is not unblinded until the end of the study unless medical treatment of the patient depends on knowing the study drug the patient received. In the event that the blind needs to be broken because of a medical emergency, the investigator may unblind an individual patient’s treatment allocation without the sponsor’s prior agreement. If a patient’s treatment allocation is unblinded, the study medical monitor must be notified within 24 hours after breaking the blind.

The treatment assignment is unblinded through IXRS. Reasons for treatment unblinding must be clearly explained and justified in the eCRF. The date on which the code was broken together with the identity of the person responsible must also be documented.

### Compliance With Study Treatment

Compliance with the study drug is assessed at each visit. Compliance is assessed based on returned tablet counts during the site visits and documented in the source documents and eCRF at the time points specified in Table 1.

A record of the number of IMB-1018972 or placebo tablets dispensed to and returned by each patient must be maintained and reconciled with the study drug and compliance records. Study treatment start and stop dates, including dates for study drug delays and/or dose reductions, are recorded in the eCRF.

### Prior and Concomitant Medications and Therapies

Use of all concomitant medications taken within 30 days before screening (Visit 1) through the safety follow-up visit (Visit 7) is recorded in the patient’s eCRF. The minimum requirement is that drug name and the dates of administration are to be recorded. This includes all prescription drugs, herbal products, vitamins, minerals, and OTC medications. Any changes in concomitant medications are also recorded in the patient’s eCRF.

Any prior use of anti-anginal medications is recorded in the patient’s eCRF, even if it was discontinued in the past. The minimum requirements are that drug name, start date, end date, and reason for discontinuation (if discontinued) is recorded.

Nitroglycerin use is reported by the patient in an electronic diary each evening for a minimum of 14 days prior to the randomization visit (Visit 3) and each evening throughout the double-blinded treatment period, as described below.

Any concomitant medication deemed necessary for the welfare of the patient during the study may be given at the discretion of the investigator. However, it is the responsibility of the investigator to ensure that details regarding the medication are recorded in full in the eCRF.

For patients outside the US, perhexiline, TMZ, or meldonium therapies within 1 month before screening through the end of the double-blinded treatment period are exclusionary.

### Study Assessments and Procedures

Before performing any study procedures, all potential patients sign an ICF. Additional procedural details related to the ICF are provided below.

Study visits and the timing of assessments are detailed in the Schedule of Events.

### Primary Efficacy Assessments

An ETT is conducted following a modified Bruce protocol at the time points specified in Table 1 and the TtSTD (as determined by a central ECG reader), TtAO (as reported by the patient), and onset of exercise-limiting angina (as reported by the patient) are recorded. The primary assessment is the TED determined by the onset of exercise-limiting angina.

Note: Intercurrent events such as impaired physical functioning due to comorbid conditions may preclude assessment of the primary efficacy endpoint. The primary efficacy endpoint is the change from baseline up to Week 8 in TED.

### Secondary and Exploratory Efficacy Assessments

Exploratory efficacy assessments includes TED, TtSTD, and TtAO obtained from the modified Bruce protocol ETTs conducted at the time points specified in Table 1.

The occurrence of angina episodes and NTG use during the screening period and double-blinded treatment period is reported by the patient as specified in Table 1.

A wearable activity monitor is dispensed to each patient to collect activity during the screening and double-blinded treatment periods, as described in Table 1.

The CCS grading system for angina is employed to assess the severity of angina (as reported by the clinician) at the time points specified in Table 1.

The SAQ is completed to assess physical limitation, anginal stability, and treatment satisfaction at the time points specified in Table 1.

The SF-12v2 is completed to assess HRQoL at the time points specified in Table 1. The CGI-S and CGI-C are completed at the site at the time points specified in Table 1. Sample questionnaires are provided in Appendix 13.2.

The PGI-S and PGI-C are completed by the patient in an electronic diary, as specified in Table 1. Sample questionnaires are provided in Appendix 13.2.

Clinical laboratory parameters and biomarkers that reflect cardiovascular health and/or disease (including, but not limited to, CRP, HbA1c, NT-proBNP, troponin, and antifibrotic biomarkers) are measured at the time points specified in Table 1.

### Adverse Events

The investigator is responsible for reporting all AEs that are observed or reported during the study, regardless of their relationship to the study drug or their clinical significance.

An AE is defined as any untoward medical occurrence in a patient enrolled into this study regardless of its causal relationship to the study drug. Patients are instructed to contact the investigator at any time after randomization if any symptoms develop.

A TEAE is defined as any event not present before exposure to the study drug or any event already present that worsens in either intensity or frequency after exposure to the study drug.

### Serious Adverse Events

An SAE is defined as any event that
results in death
is immediately life threatening
requires inpatient hospitalization or prolongation of existing hospitalization
results in persistent or significant disability/incapacity
is a congenital anomaly/birth defect

Important medical events that may not result in death, be life threatening, or require hospitalization may be considered SAEs when, based upon appropriate medical judgment, they may jeopardize the patient or may require medical or surgical intervention to prevent one of the outcomes listed in this definition. Examples of such medical events include allergic bronchospasm requiring intensive treatment in an emergency room or at home, blood dyscrasias or convulsions that do not result in inpatient hospitalization, or the development of drug dependency or drug abuse.

### Suspected Unexpected Adverse Events

A SUSAR is defined as an adverse reaction, the nature or severity of which is not consistent with the applicable product information (eg, investigator’s brochure).

### Eliciting and Documenting Adverse Events

Adverse events are assessed from the time the patient signs the ICF until exit from the study (up to 14 days after the last dose of study drug). Any SAE that occurs after these time periods and that is believed to be related to study drug should be reported by the investigator (as described in Section 6.2.1.3).

At every study visit, patients is asked a standard nonleading question to elicit any medically-related changes in their well-being. They are also asked if they have been hospitalized, had any accidents, used any new medications, or changed concomitant medication regimens (both prescription and OTC medications).

In addition to patient observations, AEs identified from any study data (eg, laboratory values, physical examination findings, ECG changes) or identified from review of other documents (eg, patient electronic diary) that are relevant to patient safety are documented on the AE page in the eCRF.

### Assessment of Severity

The severity, or intensity, of an AE refers to the extent to which an AE affects the patient’s daily activities. The intensity of the AE is rated as mild, moderate, or severe using the following criteria:

Mild: These events require minimal or no treatment and do not interfere with the patient’s daily activities.

Moderate: These events result in a low level of inconvenience or concern with the therapeutic measures. Moderate events may cause some interference with normal functioning.

Severe: These events interrupt a patient’s usual daily activity and may require systemic drug therapy or other treatment. Severe events are usually incapacitating.

Changes in the severity of an AE should be documented to allow an assessment of the duration of the event at each level of intensity to be performed. Adverse events characterized as intermittent do not require documentation of onset and duration of each episode.

### Assessment of Causality

The investigator’s assessment of an AE’s relationship to the study drug is part of the documentation process, but it is not a factor in determining what is or is not reported in the study. If there is any doubt as to whether a clinical observation is an AE, the event should be reported.

The relationship or association of the test article in causing or contributing to the AE is characterized using the following classification and criteria:
Unrelated: There is no association between the study drug and the reported event.
Possible: Treatment with the study drug caused or contributed to the AE, ie, the event follows a reasonable temporal sequence from the time of drug administration or follows a known response pattern to the study drug, but could also have been produced by other factors.
Probable: A reasonable temporal sequence of the event with drug administration exists and, based upon the known pharmacological action of the drug, known or previously reported adverse reactions to the drug or class of drugs, or judgment based on the investigator’s clinical experience, the association of the event with the study drug seems likely. The event disappears or decreases on cessation or reduction of the dose of study drug.
Definite: A definite causal relationship exists between drug administration and the AE, and other conditions (concurrent illness, progression/expression of disease state, or concurrent medication reaction) do not appear to explain the event. The event reappears or worsens if the study drug is readministered.

### Reporting Adverse Events

All AEs reported or observed during the study are recorded on the AE page in the eCRF. Information to be collected may include, but is not limited to, the following:
drug treatment
dose
event term
date and time of onset
investigator-specified assessment of severity and relationship to the study drug
date and time of resolution of the event
seriousness
any required treatment or evaluations
outcome
action taken with study drug

Adverse events resulting from concurrent illnesses, reactions to concurrent illnesses, reactions to concurrent medications, or progression of disease states must also be reported. All AEs are followed to adequate resolution. The MedDRA is used to code all AEs.

Any medical condition that is present at the time that the patient is screened but does not deteriorate should not be reported as an AE. However, if it deteriorates at any time during the study, it should be recorded as an AE.

### Follow-Up of Patients Reporting Adverse Events

All AEs must be reported in detail on the appropriate page in the eCRF and followed to satisfactory resolution, until the investigator deems the event to be chronic or not clinically significant, the event is considered to be stable, or the patient is lost to follow-up.

### Physical Examinations

Physical examinations is performed at the time points specified in Table 1.

A complete physical examination includes an evaluation of body systems, including but not limited to the following: cardiovascular, respiratory, gastrointestinal, and neurological systems. The investigator should pay special attention to clinical signs related to previous serious illnesses.

Height (cm) and weight (kg) are also measured and recorded at the time points specified in Table 1. Weight and height are measured with shoes off and preferably with the same balance at each visit

### Vital Signs

Vital signs including temperature (oral, tympanic, or skin), pulse, respiratory rate, and systolic and diastolic blood pressure) are assessed at the time points specified in Table 1.

Vital signs are measured in a semi-supine position after 5-minutes rest and before any 12-lead ECG assessment or blood collection for laboratory tests is performed. Two readings of blood pressure and pulse are taken and averaged to give the measurement to be recorded in the eCRF.

### Electrocardiograms

Three types of ECGs, including standard rest ECG (supine), torso ETT rest ECG (standing), and exercise ECG (standing) are obtained at the time points specified in Table 1.

Eligibility related ECG/ETT tracings should be faxed to the core ECG laboratory for assessment of eligibility allowing rapid eligibility turnaround, but the original ECG tracings should also be shipped to the core ECG laboratory for final analysis in follow-up. Copies of the tracings should be retained at the site, and the original rest and exercise ECG tracings should be sent to the core ECG laboratory for final analysis. For all other ECGs obtained in the study, the original tracings should be sent to the core ECG laboratory and copies should be retained at the site.

Prior to each modified Bruce protocol treadmill ETT during the screening period, at Visits 5 and 6 (Weeks 8 and 16), and within 14 days of study termination a standard rest 12-lead ECG with electrodes in the standard position (NOT TORSO LEADS) with visible calibration pulse, and paper speed of 25 mm/sec are obtained. All standard rest ECG tracings should be included with the eligibility ETT tracings for the core ECG/ETT laboratory to review.

If after randomization, a patient is admitted to hospital for a cardiac event, sites should obtain the initial hospitalization, emergency room records, discharge summary, and rest ECG tracings associated with the event. The original (hard copy) ECG tracings should be sent to the core ECG laboratory and the event packet with ECG’s included to the DCC. Copies should be retained by the site.

### Modified Bruce Protocol Treadmill Exercise Tolerance Test

Modified Bruce protocol treadmill ETTs are performed at the time points specified in Table 1.

The only exercise protocol that is accepted for this trial is the modified Bruce protocol, modified with the insertion of 2 three-minute warm-up stages. The first stage is 3 minutes at 1.7 mph (2.74 km/h) and 0% grade; the second stage is 3 minutes at 1.7 mph (2.74 km/h) and 5% grade; the third stage is 1.7 mph (2.74 km/h) and 10% grade (standard stage 1 of the Bruce protocol [approximately 5 METS]).

A patient who can exercise >9 minutes on this protocol during the eligibility phase of the study should not be randomized into the study. All antianginal medications (including IMB-1018972 or placebo) should be withheld on the morning of the ETTs. It is preferred that over the duration of the study, each individual patient’s ETT be performed on the same equipment, under the same conditions, and with the same exercise technician. All eligibility ETTs require confirmation by the core ECG/ETT laboratory before the patient can be randomized. Eligibility related ETT tracings should be faxed to the core ECG laboratory for assessment of eligibility allowing rapid eligibility turnaround, but the original ETT tracings should also be shipped to the core ECG laboratory for final analysis in follow-up. Copies of the tracings should be retained at the site, and the original tracings should be sent to the core ECG laboratory for final analysis. For all randomized patients, the ORIGINAL ETT tracings at baseline (Visit 1), and at Weeks 8 and 16 (Visits 5 and 6) should be sent to the core ECG laboratory, and copies should be retained at the site for their records.

### Actigraphy

Patients enrolled in the study are asked to wear an activity monitor for 24 hours a day (except as specified in the Actigraph patient guide and while uploading activity data or recharging the device) to measure their movement from the first screening visit (Visit 1) through Visit 6. The patient should wear the activity monitor for a minimum of 15 hours per day for a day to be considered compliant. The site should monitor compliance and reeducate the patient if compliance is less than 15 hours per day.

The activity monitor are dispensed to the patient at Visit 1 and the patient is trained on the device and instructed to wear the activity monitor on their nondominant wrist for the duration of the study (the screening period plus the double-blinded treatment period, for a total of 18 to 20 weeks). The patient is also be assigned a data hub that serves to upload data and charge the activity monitor at home (as specified in the Actigraph patient guide). The site should upload activity data at the scheduled visits to further ensure appropriate upload and transfer of data from the activity monitor. During the uploading time, the patient is not wearing the activity monitor. At Visit 6, the patient returns the activity monitor and data hub for a final data transfer and completion of the activity assessment.

### Pregnancy

Serum and urine pregnancy tests are performed at the time points specified in Table 1.

Pregnancy is not regarded as an AE unless there is a suspicion that a study drug may have interfered with the effectiveness of a contraceptive medication. Study drug should be discontinued immediately in case of pregnancy. Any pregnancy that occurs during study participation must be reported as an SAE as described below.

To ensure patient safety, each pregnancy must be reported to the sponsor within 24 hours of learning of its occurrence. The pregnancy must be followed-up to determine outcome (including spontaneous miscarriage, elective termination, normal birth, or congenital abnormality) and status of mother and child, even if the patient was discontinued from the study. Pregnancy complications and elective terminations for medical reasons must be reported as an AE or SAE. Spontaneous miscarriages must be reported as an SAE.

Any SAE occurring in association with a pregnancy, brought to the investigator’s attention after the patient has completed the study, and considered by the investigator as possibly related to the study drug, must be promptly reported to the sponsor.

### Contraception Guidance

Patients of child-bearing potential must be willing to practice an approved method of birth control for the duration of the study and until 90 days after the last dose of study drug.

A female patient is eligible to participate if she is of nonchild-bearing potential, defined as premenopausal females with permanent sterilization (includes hysterectomy, bilateral oophorectomy, or bilateral salpingectomy in a female patient of any age); or postmenopausal, defined as no menses for 12 months without an alternative medical cause; or, if of child-bearing potential, if she is using a highly effective method for avoidance of pregnancy and continues to use these methods for the duration of the study and until 90 days after the last dose of study drug.

Male patients must agree to use one of the contraception methods listed below for the duration of the study and until 90 days after the last dose of study drug.

The following is the all-inclusive list of the highly effective methods (per the CTFG guidance) for avoiding pregnancy in females of child-bearing potential (ie, the methods have a failure rate of less than 1% per year when used consistently and correctly and, when applicable, in accordance with the product label) (Hatcher et al 2007):
Complete abstinence from penile-vaginal intercourse, when this is the preferred and usual lifestyle
Oral contraceptive, either combined or progestogen alone
Injectable progestogen
Implants of etonogestrel or levonorgestrel
Estrogenic vaginal ring
Percutaneous contraceptive patches
Intrauterine device or intrauterine system
Tubal sterilization
Essure micro-insert system (provided confirmation of success 3 months after procedure) Male partner sterilization (vasectomy with documentation of azoospermia) prior to the female patient’s entry into the study, and this male is the sole partner for that patient. The information on the male sterility can come from the site personnel’s review of the patient’s medical records; medical examination of the patient, and/or semen analysis; or interview with the patient on his medical history. Sterilized males (vasectomy with documentation of azoospermia) must use condoms until 90 days after last dose of study drug.

### Laboratory Analyses

Samples for clinical laboratory tests (hematology, serum chemistry, coagulation, lipid profile, and urinalysis), pregnancy testing, serology testing, drug screen, plasma PK, and analysis of clinical laboratory parameters and biomarkers that reflect cardiovascular health and/or disease are collected at the time points specified in Table 1. There is no requirement for fasting prior to sampling. Processing, storage, and shipping procedures for all clinical laboratory samples are provided in a laboratory manual.

Any abnormal laboratory test results (hematology, serum chemistry, coagulation, lipids, or urinalysis) or other safety assessments (eg, ECGs, physical examination findings, vital sign measurements), including those that worsen from baseline and/or are clinically significant in the medical and scientific judgment of the investigator are to be recorded as AEs or SAEs. Clinically significant abnormal laboratory test results are reviewed by the investigator and recorded as AEs or SAEs on the appropriate page in the eCRF.

Blood and other biological samples are collected for the laboratory tests indicated in Table 2.

TABLE 2

| | |
|---|---|
| Hematology | Hematocrit, hemoglobin, red blood cell count, white blood cell count, platelet count, mean corpuscular hemoglobin, mean corpuscular hemoglobin concentration, mean corpuscular volume, mean platelet volume, red cell distribution width, nucleated red blood cells Differential blood counts, including basophils, eosinophils, immature granulocyte, lymphocytes, monocytes, and neutrophils |
| Serum chemistry | Alkaline phosphatase, alanine aminotransferase, aspartate aminotransferase, total bilirubin, calcium, chloride, creatinine, gamma glutamyl transferase, random glucose, lactate dehydrogenase, phosphorous, potassium, total protein, sodium, blood urea nitrogen |
| Coagulation | International normalized ratio, prothrombin time, partial thromboplastin time |
| Lipid profile | High-density lipoprotein cholesterol, low-density lipoprotein cholesterol |
| Urinalysis | Appearance, color, bilirubin, blood, glucose, ketones, leukocyte esterase, nitrites, pH, protein, specific gravity, urobilinogen If there is a clinically significant positive result, urine is sent for microscopy and/or culture. |
| Pregnancy | Serum f-hCG (for female patients of child-bearing potential), serum follicle-stimulating hormone (suspected postmenopausal female patients only), urine pregnancy test |
| Serology testing | HBsAg, HCV antibody, HCV viral load (confirmatory for HCV antibodypositive patients), HIV-1/HIV-2 antibodies, genius HIV1/2 differentiation (confirmatory for HIV1/2 antibody-positive patients) |
| Urine drug screen | Opiates, methadone, cocaine, amphetamines (including ecstasy), barbiturates |
| Plasma PK | IMB-1028814 and TMZ |
| Other assessments of cardiovascular health/disease (including but not limited to) | CRP, HbA1c, NT-proBNP, troponin, antifibrotic biomarkers |

The investigator must review the laboratory reports, document this review, and record any clinically relevant changes occurring during the study. If the laboratory reports are not transferred electronically, the values must be filed with the source information (including reference ranges). In most cases, clinically significant abnormal laboratory findings are those that are not associated with the underlying disease, unless judged by the investigator to be more severe than expected for the patient’s condition.

All laboratory tests with values considered clinically significantly abnormal during participation in the study or within 14 days after the last dose of study drug should be repeated until the values return to normal or baseline or are no longer considered clinically significant by the investigator or study medical monitor. If such values do not return to normal/baseline within a period of time judged reasonable by the investigator, the etiology should be identified and the diagnosis should be recorded as the AE, and the sponsor should be notified.

All protocol-required laboratory assessments must be conducted in accordance with the laboratory manual and the Schedule of Events (Table 1). If laboratory values from nonprotocol-specified laboratory assessments performed at the institution’s local laboratory require a change in patient management or are considered clinically significant by the investigator (eg, SAE or AE or dose modification), then the results must be recorded in the eCRF.

### Sample Collections

Samples are collected at the time points specified in Table 1. Processing, storage, and shipping procedures for all clinical laboratory, PK, and biomarkers samples are provided in a laboratory manual.

Blood collection for plasma PK and exploratory analysis of clinical laboratory parameters and biomarkers of cardiovascular health/disease can be performed either in the fasted or fed state.

The time of PK blood sampling and the time of the last dose of study drug are recorded in the eCRF. All antianginal medications (including IMB-1018972 or placebo) should be withheld on the morning of Visits 5 and 6 to obtain trough levels at the time of the ETT. All anti-anginal medications (including IMB-1018972 or placebo) should be taken as usual in the morning of Visit 4.

### Statistical Considerations

### Primary Estimand

The primary comparison of interest is the difference between treatments in TED from the modified Bruce protocol treadmill ETT, which assesses functional capacity. The targeted patient population consists of patients with refractory angina who took at least 1 dose of study drug, regardless of compliance to the treatment regimen or changes in concomitant medication. Inferential analysis of the primary comparison is conducted with change from baseline to Week 8 (or early termination, if prior to Week 8) in TED as the primary endpoint, summarized as an average and according to randomized treatment group. Intercurrent event types of special interests are summarized in Table 3.

TABLE 3

| | |
|---|---|
| Label | Intercurrent Event Type |
| IcEv1 (NTG) | Rescue medication (NTG) taken as required (in addition to the study drug) |
| IcEv2 (Anti-angina) | Change in background antianginal medication |
| IcEv3 (Discontinue) | Discontinuation of study drug due to AEs |
| IcEv4 (Death) | Death |

A summary of the primary estimand is presented in Table 4.

TABLE 4

| | |
|---|---|
| Estimand Label | Estimand 1 |
| Estimand Description | Mean difference between treatments in change from baseline to Week 8 (or early termination, if prior to Week 8) in TED, irrespective of changes in frequency of NTG, or background of antianginal concomitant medication, or duration of treatment |
| Target Population | Patients with refractory angina who would be on a stable antianginal regime for at least 1 month prior to the first screening ETT and meet the study entry criteria |
| Endpoint | Change from baseline up to Week 8 in TED |
| Treatment Condition(s) | Test: IMB-1018972 + standard care Reference: Placebo + standard care |
| Population-Level Summary | Mean difference |
| Intercurrent Event | |
| Strategy | |
| IcEv1(NTG) | Treatment policy |
| IcEv2 (Anti-angina) | Treatment policy |
| IcEv3 (Discontinue treatment) | Treatment policy |
| IcEv4 (Death) | While on treatment |
| Rationale for Strategies | Treatment policy strategy is used to understand the treatment effect over and above standard of care; while on treatment strategy is used when observations after death do not exist. |

### Primary Efficacy Endpoint

Change from baseline to Week 8 (or early termination, if prior to Week 8) in TED. For simplicity, the primary endpoint is referred to as change from baseline up to Week 8 in TED.

### Secondary Efficacy Endpoints

The secondary efficacy endpoints include the following:
Change from baseline in frequency of angina per week at Week 16 (during 14 days prior to Visit 6)
Change from baseline in frequency of rescue NTG use at Week 16 (during 14 days prior to Visit 6)
Change from baseline in total daily activity at Week 16 (during 14 days prior to Visit 6)
Change from baseline up to Week 8 in TtAO
Change from baseline up to Week 8 in TtSTD
Overall concordant domain responder (responder on 2 or more individual domains with no worsening)

### Exploratory Efficacy Endpoints

The exploratory endpoints include the following:
Change from baseline in TED, TtSTD, and TtAO at Week 16
Incremental change in TED between Week 8 and Week 16
TED, TtSTD, and TtAO predicted by IMB-1018972 trough PK concentration at Weeks 8 and 16
Change from baseline in the TS scale at Weeks 8 and 16
SAQ-AS and SAQ-PL at Weeks 8 and 16
SF-12v2 at Weeks 8 and 16
CGI-S and CGI-C at Weeks 8 and 16
PGI-S and PGI-C at Weeks 8 and 16
CCS grade at Weeks 8 and 16
ETT responder (5% improvement from baseline up to Week 8)
SAQ-AS or SAQ-PL responder (10-point improvement on either scale at Weeks 8 and 16)
CCS angina grade responder (improvement of one of more angina grades at Week 8 and 16)
Change from baseline in CRP at Weeks 8 and 16
Change from baseline in HbA1c and HbA1c characterization (normal/high) levels at Weeks 8 and 16
Change from baseline in NT-proBNP at Weeks 8 and 16
Change from baseline in troponin at Weeks 8 and 16
Change from baseline in antifibrotic biomarkers and other potential biomarkers at Weeks 8 and 16
Change from baseline in peak RPP at Weeks 8 and 16
Change from baseline in RPP at time of angina onset at Weeks 8 and 16
Change from baseline in RPP at time of STD >0.5 mm at Weeks 8 and 16
Plasma concentrations of the IMB-1018972 metabolites IMB-1028814 and TMZ at Weeks 8 and 16 for exposure-PD effects analyses
Actigraphy compliance

### Sample Size Determination

A clinically meaningful treatment difference at the group level is assumed to range from 30 to 32 seconds and the variability (ie, standard deviation) of the primary endpoint (change from baseline up to Week 8 in TED) is assumed to range from 112 to 124 seconds. A clinical trial of approximately 300 evaluable patients per treatment group has at least 90% statistical power if the population treatment difference is 32 seconds and the standard deviation is 112 seconds, whereas the clinical trial has at least 80% statistical power if the population treatment difference is 30 seconds and the standard deviation is 124 seconds.

These sample size calculations are based on a 2-sided testing procedure with a Type I error rate of 5%.

### Analysis Sets

The following analysis sets are used in the statistical analyses.
Enrolled population: The enrolled set consists of all patients who sign the ICF.
Randomized population: The randomized population consists of all patients who are randomized.
Intent-to-Treat Population (ITT): The ITT consists of patients randomly assigned to study intervention and who take at least 1 dose of study intervention; patients are analyzed according to the intervention as assigned.
Evaluable Population: The evaluable population consists of all qualified patients (per disease-defining entry criteria) randomly assigned to study intervention and who take at least 80% of the assigned study intervention (total count/total expected) prior to collection of their end of study ETT; patients are analyzed according to the intervention they actually received.
Safety Population: The safety population consists of patients randomly assigned to study intervention and who take at least 1 dose of study intervention; patients are analyzed according to the intervention they actually received.

The efficacy analyses are performed using the ITT population.

### Description of Subgroups to Be Analyzed

Primary and secondary endpoints used in the serial gatekeeping procedure are summarized by stratification factors.

### Statistical Analysis Methodology

Descriptive statistics (eg, number of patients, mean, standard deviation, median, minimum, maximum, 25th and 75th percentile) are calculated for continuous variables and presented by treatment group. For summaries of continuous PK parameters, the coefficient of variation is also presented. For continuous endpoints (eg, absolute change from baseline and percent change from baseline), an ANCOVA model is fitted with treatment group and stratification factors as categorical variable terms and the corresponding baseline efficacy value as a covariate, unless otherwise specified. Least squares means and 95% confidence intervals for each treatment group as well as the LSM difference between the treatment groups are presented, along with the corresponding confidence intervals and P values. The CDF of change from baseline will be plotted by treatment group. To aid in the interpretation of the graphical representation of the CDF across treatment groups, a two-sample Kolmogorov-Smirnov are conducted.

For categorical variables, frequencies and percentages for each category is presented by treatment group. For responder endpoints, the proportion of responders is compared between treatment groups using a CMH test controlling for stratification factors. The difference in the proportion of responders between the treatment groups as well as the CMH estimates of odds ratio and P values is presented, along with the corresponding confidence intervals.

Baseline for frequency of angina, angina-free days, frequency of NTQ and total daily activity is derived using the data of 14 days prior to the randomization visit (Visit 3). Baseline for all other endpoints is defined as the last non-missing value measured before the randomization visit (Visit 3), unless otherwise specified. All summaries are presented by treatment group, unless specified otherwise. All hypothesis tests are two-sided with a 5% significance level, and 95% confidence intervals are used, unless otherwise specified.

All statistical analyses are performed using SAS software Version 9.4 (or higher).

### Statistical Hypothesis and Multiple Comparison Procedure

The null hypothesis is that there is no difference between treatments in TED, which assesses functional capacity, employing change from baseline to Week 8 (or early termination, if prior to Week 8) as the primary endpoint.

The alternate hypothesis is that there is a difference between treatments in TED, which assesses functional capacity, employing change from baseline to Week 8 (or early termination, if prior to Week 8) as the primary endpoint.

Inferential testing to compare treatment groups proceed using a serial gatekeeping procedure with a hierarchical structure while controlling the family-wise error rate at 5% (a = 0.05).

The first family consists of a single primary endpoint (ie, change from baseline up to Week 8 in TED). If the primary endpoint reaches statistical significance (P<0.05), then the study is considered positive for efficacy. However, if the primary endpoint does not reach statistical significance, no further tests are interpreted inferentially.

Subsequent testing after the first family (“gate”) proceeds in a similar manner where the second family consists of the first secondary endpoint (ie, change from baseline in frequency of angina per week at Week 16) and so forth. Thereby, the global family-wise error rate is maintained at a = 0.05.

The prespecified order of the primary and secondary endpoints in the serial-gatekeeping, multiple-comparison procedure is described in Table 5:

TABLE 5

| | | | |
|---|---|---|---|
| Family | Domain | Endpoint | Method |
| 1 - Primary | Functional Capacity | Change from baseline up to Week 8 in TED | MMRM |
| 2 - Secondary | Symptom Severity | Change from baseline in frequency of angina per week at Week 16 (during 14 days prior to Visit 6) | ANCOVA |
| 3 - Secondary | Symptom Severity | Change from baseline in frequency of rescue NTG use at Week 16 (during 14 days prior to Visit 6) | ANCOVA |
| 4 - Secondary | Functional Capacity | Change from baseline in total daily activity at Week 16 (during 14 days prior to Visit 6) | ANCOVA |
| 5 - Secondary | Functional Capacity | Change from baseline up to Week 8 in TtAO | ANCOVA |
| 6 - Secondary | Functional Capacity | Change from baseline up to Week 8 in TtSTD | ANCOVA |
| 7 - Secondary | Multi-Domain | Overall Concordant Domain responder (responder on 2 or more individual domains with no worsening) | CMH test |

### Analysis of Primary Efficacy Endpoint

The primary efficacy analysis is performed using the ITT population. The observed and change from baseline values are summarized at each applicable visit. The primary efficacy analysis is conducted by first implementing the placebo-based multiple imputation procedure (Mallinckrodt and Lipkovich 2017), then perform MMRM analysis for each imputed dataset, and combine the results. The placebo-based multiple imputation imputes missing data after dropout (monotonic) for all patients based on the placebo group data. The methodology is conservative, assuming patients in the active treatment group behave like the patients in the placebo group after dropout. The MNAR mechanism is assumed for the missing data in this analysis.

### Sensitivity Analyses

To assess the robustness of the study conclusions to missing data, alternative models with different assumptions regarding missingness are employed. Firstly, the MMRM model is employed with the observed data, without imputation, essentially implementing a MAR alternative within the same modeling framework. In addition, a delta adjustment method applied to the treatment arm is implemented in the manner of the primary analysis by applying progressively larger adjustments to the treatment arm until the delta corresponding to loss of statistical significance is located (ie, a “tipping point” analysis). As with the primary analysis, the delta adjustment analysis begins with imputation at Week 8, but now with each imputation adjusted by the current delta. Implausibility of the delta corresponding to loss of significance aids clinical interpretation and be indicative of a robust primary analysis.

### Missing Data

In addition to the counts and percentage of patients enrolled and in each study population by treatment group, the probability of remaining in the study is presented in a Kaplan- Meier plot by treatment group and, in addition, by the primary reasons for discontinuation within treatment group (eg, AE, lack of efficacy, and all other reasons). Logistic regression, with a missing/observed indicator as response, is used to further explore associations between the primary endpoint and missingness.

### Analysis of Secondary Efficacy Endpoints

The secondary efficacy analyses are performed using the ITT population.

For change from baseline endpoints, an ANCOVA model as outlined below is fitted with treatment group and stratification factors as categorical variable terms and the corresponding baseline efficacy value as a covariate. Also, the observed and change from baseline values is summarized at each applicable visit.

For responder endpoints, as outlined below, the proportion of responders is compared between treatment groups using a CMH test controlling for stratification factors. The difference in the proportion of responders between the treatment groups as well as the CMH estimates of odds ratio and P values is presented, along with the corresponding confidence intervals. Also, frequencies and percentages for each category is presented by treatment group.

In addition, change from baseline in TED at Week 16 is analyzed in a similar manner of the primary endpoint.

### Analyses of Exploratory Efficacy Endpoints

The exploratory efficacy analyses are performed using the ITT population.

All endpoints are summarized using descriptive statistics described below, where appropriate. Descriptive summaries are presented for trough concentration. Scatter plots of ETT endpoints (TED, TtSTD, and TtAO) vs trough concentration are produced, along with the regression line.

### Pharmacokinetic/Pharmacodynamic Analyses

The TED, TtSTD, and TtAO are predicted by IMB-1018972 trough PK concentration at Weeks 8 and 16 as described above.

### Safety Analyses

The safety population is used for the safety data analysis unless otherwise stated.

The safety evaluation includes AEs (including AEs leading to discontinuation of study drug and AEs leading to death), clinical laboratory safety tests (eg, hematology, serum chemistry, coagulation, lipids, and urinalysis), change in vital signs, 12-lead ECG findings, physical examinations, and any other parameters deemed necessary for safety assessment including, but not limited to, the following:
Rate of death, nonfatal MIs, urgent revascularization
Number of hospitalization for cardiac or noncardiac reasons

Descriptive statistics of vital signs, 12-lead ECG parameters and clinical laboratory data are presented by treatment group in tabular form with N, arithmetic mean, standard deviation, median, minimum and maximum or in frequency tabulation form as appropriate. For continuous parameters, change from baseline are summarized by treatment group across time on study. Potentially clinically significant changes or values as well as shift analysis for vital signs, ECG, laboratory data, and physical examination are flagged in data listings and may be summarized as appropriate.

All safety data is presented in the data listings and is flagged for events of interest (eg, out of range laboratory data), as appropriate. Adverse events are coded using the current version of MedDRA and are summarized by system organ class and preferred term. Treatment-emergent events are tabulated. Events that are considered related to treatment (possibly, probably, definitely drug-related) are also tabulated. A summary is also provided that enumerates AEs by severity. Deaths, SAEs, and AEs leading to study drug discontinuation is also tabulated.

Other safety parameters are summarized, as appropriate.

### Other Analyses

Summary statistical analyses are provided for demographics, baseline characteristics including BMI, medical history, and physical examination at baseline. Also, prior and concomitant medications, and study drug are summarized.

### Example 2

### Brief Study Design

A Phase 1 first-in-human, randomized, double-blind, placebo-controlled single ascending dose and multiple ascending dose study to investigate the safety, tolerability, and pharmacokinetics (including food effect) or IMB-1018972 in healthy subjects.

### Objectives

The primary objective is to assess the safety and tolerability of single and multiple ascending oral doses of IMB-1018972, and single oral doses of trimetazidine.

Secondary objectives include: To assess the pharmacokinetic (PK) profile of single and multiple ascending oral doses of IMB-1018972, and single oral doses of trimetazidine; To assess the effect of food on the absorption and the PK profile of IMB-1018972 following a single oral dose of IMB-1018972 in healthy subjects; To evaluate the effect of food on the safety and tolerability of IMB-1018972 following a single oral dose of IMB-1018972 in healthy subjects;

### Design and Treatments

This was a double-blind, randomized, placebo-controlled study, consisting of a single ascending dose (SAD) part with integrated food effect (FE) arm, a multiple ascending dose (MAD) part, to assess the safety, tolerability, and PK of ascending single and multiple oral doses of IMB-1018972 (immediate-release [IR] formulation in the SAD and MAD parts). The study started with the SAD part.

### SAD Part (and Integrated FE Arm)

In the SAD part, 5 groups of 8 healthy subjects (6 subjects on active drug and 2 on placebo in Groups A1, A2, A3, and A4, and 8 subjects on active drug in Group A5) were included. In Groups A1, A2, A3, and A4, subjects received a single oral dose of an IR formulation of IMB-1018972 or placebo under fasted conditions (an overnight fast of at least 10 hours). In Group A5, all subjects received a single oral dose of a MR formulation of trimetazidine under fasted conditions (an overnight fast of at least 10 hours). Each subject participated in only 1 group during the study.

Subjects assigned to Group A4 also participated in the FE arm and received the same single dose of IMB-1018972 or placebo under fed conditions (Food and Drug Administration [FDA]-defined high-fat breakfast after an overnight fast of at least 10 hours) in a second period at least 1 week after drug administration under fasted conditions in the SAD part.

The following treatments were administered in the SAD part under fasted conditions:
Group A1: single oral dose of 50 mg IR formulation of IMB-1018972 (n=6) or matching placebo (n=2) on Day 1
Group A2: single oral dose of 150 mg IR formulation of IMB-1018972 (n=6) or matching placebo (n=2) on Day 1
Group A3: single oral dose of 400 mg IR formulation of IMB-1018972 (n=6) or matching placebo (n=2) on Day 1
Group A4: single oral dose of 150 mg IR formulation of IMB-1018972 (n=6) or matching placebo (n=2) on Day 1 (FE group)
Group A5: single oral dose of 35 mg MR formulation of trimetazidine (Vastarel; n=8) on Day 1

The following treatment was administered in the FE arm under fed conditions (FDA-defined high-fat breakfast):
Group A4: single oral dose of 150 mg IR formulation of IMB-1018972 (n=5) or matching placebo (n=2) on Day 1 (same dose as in SAD part)

IMB-1018972 dose-escalation was based on the available safety, tolerability, and PK results of at least 5 dosed subjects in the preceding group. A dose-escalation meeting was held between the Investigator and the Sponsor. Further, a dose-escalation report (DER) was provided by the Investigator to the Independent Ethics Committee (IEC) following completion of each dose level. Escalation to the next higher dose only proceeded when none of the stopping criteria had been reached and if the available safety, tolerability, and PK results (results up to 48 hours postdose) of at least 5 dosed subjects in the preceding group were acceptable to the Investigator and the Sponsor and after a statement of no objection of the DER from the IEC.

In this first-in-human (FIH) study, the subjects participating at the lowest dose level, subjects of Group A1, were dosed according to a sentinel dosing design to ensure optimal safety. This means that initially, 2 subjects were dosed: 1 subject with IMB-1018972 and 1 subject with placebo. Since the safety and tolerability results of the first 24 hours following dosing for the initial 2 subjects were acceptable to the Investigator, the other 6 subjects (5 active drug and 1 placebo) of the lowest dose level were also dosed.

### MAD Part

In the MAD part, 2 groups of 12 healthy subjects (9 subjects on active drug and 3 on placebo in each group) were included. Subjects received multiple oral doses of an IR formulation of IMB-1018972 or placebo once every 12 hours (q12h) for 14 consecutive days. Each subject participated in only 1 group during the study.

The following treatments were administered under fed conditions as determined based on the results of Group A4 in the FE arm. The doses were selected based upon the safety, tolerability, and PK data from the SAD part:
Group B1 multiple oral doses of an IR formulation of 150 mg IMB-1018972 (n=9) or matching placebo (n=3) twice daily (q12h) for 14 days; on Day 14 only a single morning dose was administered
Group B2: multiple oral doses of an IR formulation of 50 mg IMB-1018972 (n=9) or matching placebo (n=3) q12h for 14 days; on Day 14 only a single morning dose was administered

IMB-1018972 dose escalation was based on the available safety, tolerability, and PK results of at least 8 dosed subjects in the preceding group. A dose-escalation meeting was held between the Investigator and the Sponsor. Further, a DER was provided by the Investigator to the IEC following completion of each dose level. Escalation to the next higher dose only proceed when none of the stopping criteria had been reached and if the available safety, tolerability, and PK results (results up to 48 hours after the final morning dose on Day 14) of at least 8 dosed subjects in the preceding group were acceptable to the Investigator and the Sponsor and after a statement of no objection of the DER from the IEC.

### Study Schedule

Screening: Between Day -35 and Day -1 (admission)
Confinement period: SAD part: 1 period in the clinic from Day -1 (admission) to approximately 48 hours after study drug administration (Day 3); an exception was Group A4 also participating in the FE arm in which subjects were in the clinic for 2 periods, each being from Day -1 (admission) to approximately 48 hours after study drug administration (Day 3) MAD part: 1 period in the clinic from Day -1 (admission) to approximately 48 hours after the last study drug administration on Day 14 (Day 16);
Follow-up: SAD part: 7 to 14 days after the last PK blood sample (between Day 10 and Day 17); FE arm: 7 to 14 days after the last PK blood sample in the second period (between Day 10 and Day 17); MAD part: 7 to 14 days after the last PK blood sample (between Day 23 and Day 30);

### Subjects

SAD part: 40 healthy male or female subjects (this included 8 subjects also participating in the FE arm); from Group A4 onwards, all efforts were made to have a ratio of 50:50 for male and female subjects per group, but at minimum at least 3 subjects of each gender were dosed per group
MAD part: 24 healthy male or female subjects; for each group, all efforts were made to have a ratio of 50:50 for male and female subjects, but at minimum at least 4 subjects of each gender were dosed per group

### Main Criteria for Inclusion

Age: 18 years to 65 years, inclusive, at screening
Body mass index (BMI): 18.0 kg/m² to 32.0 kg/m², inclusive
Status: Healthy subjects

### Study Drug

### Active Medication

Drug product: IMB-1018972
Activity: Fatty acid oxidation inhibitor
In development for: Ischemic cardiovascular disease
Strength: 25 mg, 100 mg, and 200 mg IR formulations (based on free base)
Dosage form: Oral IR capsule(s) to be used in the SAD and MAD parts
Manufacturer: Pharmacy at PRA
Batch number: 2479-1810-00441 (drug substance)

### IMB-1018972 Placebo (Visually Matching Active Medication)

Active substance: Not applicable
Activity: Not applicable
Strength: Not applicable
Dosage form: Oral capsule(s)
Manufacturer: Pharmacy at PRA
Batch number: Not applicable

### Active Medication

Drug product: Vastarel MR (trimetazidine dihydrochloride)
Activity: Fatty acid oxidation inhibitor
In development for: Angina pectoris
Strength: 35 mg
Dosage form: Oral modified-release tablet
Manufacturer: Servier Research & Pharmaceuticals (Pakistan) (Pvt.) Ltd.
Batch number: 273782 (drug product)

### Variables

Safety: Adverse events, clinical laboratory, vital signs, 12-lead electrocardiogram, continuous cardiac monitoring (telemetry), and physical examination
Pharmacokinetics: Plasma concentrations of IMB-1018972, IMB-1028814, and trimetazidine Urine concentrations of IMB-1018972, IMB-1028814, and trimetazidine (SAD part only) Plasma PK parameters estimated using noncompartmental analysis, as appropriate. SAD part, integrated FE arm: Cmax, tmax, AUC₀₋ₜ, AUC_{0-inf}, %AUCₑₓₜᵣₐ, kₑₗ, t_{½}, CL/F (IMB-1028814 only), and V_{z}/F (IMB-1028814 only). Day 1 of MAD part: Cₘₐₓ, tₘₐₓ, and AUC_{0-T}. Day 14 of MAD part: Cmax, tmax, Cmin, kₑₗ, tl/2, AUC_{0-T}, CLₛₛ/F (IMB-1028814 only), V_{z}/F (IMB-1028814 only), and R_{ac} Urine PK parameters estimated using noncompartmental analysis, as appropriate: Aeᵤᵣᵢₙₑ, Feᵤᵣᵢₙₑ, and CL_{R}

### Statistical Methods

Sample size calculation: For this FIH study, no prospective calculations of statistical power were made. The sample size was selected to provide information on safety, tolerability, and PK following single and multiple doses of IMB-1018972, single doses of trimetazidine, and is typical for a FIH study. Any p-values to be calculated according to the statistical analysis plan were interpreted in the perspective of the exploratory character of this study.
Safety parameters: Descriptive statistics
PK parameters: Descriptive statistics for all relevant PK parameters: n, mean, SD, minimum, median, maximum, geometric mean, and coefficient of variation; analysis of variance on Cₘₐₓ and AUC parameters to determine dose proportionality and FE

### Results

### Subject Disposition

Of the 220 subjects who were screened, 88 subjects were included in the study and received the study drug. Sixty-six subjects received a dose of IMB-1018972, 8 received trimetazidine, and 14 received placebo. Eighty-five of 88 subjects completed the study. One subject of the FE arm Group A4 withdrew consent on Day 1 of the second period after receiving the single oral dose of 150 mg IMB-1018972 under fed conditions. Another subject of the FE arm Group A4 was withdrawn from the study due to a serious adverse event (SAE) of influenza like illness (of moderate severity and unlikely related) in the first period and only received the single oral dose of 150 mg IMB-1018972 under fasted conditions and not the fed dose in the second treatment period. None of these discontinued subjects were replaced. All 88 subjects were included in the PK and safety sets.

TABLE 6

Disposition of Subject

| | Number of subjects |
|---|---|
| Screened volunteers | 220 |
| Screening failures Approved but not receiving study drug Reserve Group full Personal reasons Group cancelled Rejected in clinic Illness of volunteer Subjects receiving at least 1 dose of study drug Any dose of IMB-1018972 Placebo dose Trirnetazidine dose | 78 54 24 9 9 8 3 1 88 66 14 8 |
| Discontinued subjects | |
| Adverse event Withdraw by subject Completed subjects | 2 1 85 |

### Demographics

### SAD Part (and Integrated FE Arm)

### IMB-10108972 and Placebo

Thirty-two subjects were included of whom 23 were female and 9 were male. Mean age ranged between 29 and 46 years and mean BMI ranged between 23.0 and 26.6 kg/m2 over all treatments, including placebo. Individual age ranged between 18 and 65 years and individual BMI ranged between 19.5 and 30.3 kg/m2. Twenty-nine subjects were of white race, 1 subject was Asian, 1 subject was Black or African American, and 1 subject was Native Hawaiian or Other Pacific Islander.

### Trimetazidine Group

Eight subjects were included of whom 5 were female and 3 were male. Mean age was 32 years and mean BMI was 23.7 kg/m2. Individual age ranged between 20 and 65 years and individual BMI ranged between 19.4 and 26.7 kg/m2. Seven subjects were of white race and 1 subject was of multiple race.

### MAD Part

Twenty-four subjects were included of whom 12 were female and 12 were male. Mean age ranged between 38 and 44 years and mean BMI ranged between 25.2 and 26.7 kg/m2 over all treatments, including placebo. Individual age ranged between 18 and 64 years and individual BMI ranged between 19.1 and 30.9 kg/m2. Eighteen subjects were of white race, 2 subjects were of multiple race, 2 subjects were American Indian or Alaska Native, 1 subject was Asian, and 1 subject was Black or African American.

### Safety

In the SAD part, treatment with single oral doses of 50 mg, 150 mg, and 400 mg IMB-1018972 under fasted conditions, treatment with single oral doses of 150 mg IMB-1018972 under fed conditions, and treatment with single oral doses of 35 mg trimetazidine were well tolerated by healthy male and female subjects. During the SAD part, the most common AEs were 6 TEAEs of flushing (reported terms were ‘niacin flush’ and ‘flushing neck’), of which 5 TEAEs were of moderate severity and 1 TEAE was of mild severity. Four subjects reported flushing after a single dose of 400 mg IMB-1018972 under fasted conditions, and 2 subjects of the FE arm reported flushing after a single dose of 150 mg IMB-1018972 under fasted conditions. These TEAEs were all considered by the Investigator to be related to the study drug. No subjects dropped out due to flushing and flushing was not considered a safety issue. There were no clinically important trends in the physical examinations, vital signs, clinical laboratory, or ECG results. Dose escalation beyond 400 mg IMB-1018972 IR did not proceed as planned based on the PK exposure levels of IMB-1028814 and trimetazidine exceeding the target exposure levels in the 400 mg group and the findings of flushing at that dose. The predefined target exposure level was approximately 3 to 4 ‘trimetazidine equivalents’, ie, the ratio of the combined exposure of the active metabolites of IMB-1018972 to the single oral doses of 35 mg MR trimetazidine as seen in published literature.

In the MAD part, 14-day treatment with oral q12h doses of 50 mg and 150 mg IMB-1018972 under fed conditions was well tolerated by healthy male and female subjects. The most common AEs were 7 incidental and mild TEAEs of flushing that occurred in 6 subjects who had received 150 mg IMB-1018972 q12h. Five of these 6 subjects reported only a single TEAE of flushing during the 14 days dosing period. One subject reported flushing twice, on Day 2 and on Day 14. No TEAEs of flushing were reported following administration of 50 mg IMB-1018972 q12h. No subjects dropped out and no modification of the dose was needed due to the TEAEs of flushing.

Overall, no deaths were reported during the study. The majority of the reported TEAEs were transient and resolved without sequelae by follow-up. Most TEAEs were of mild severity and no severe TEAEs were reported during the study. TEAEs of moderate severity were the 5 TEAEs of flushing mentioned above and 1 TEAE each of restlessness, back pain, nausea, tonsillitis, post procedural hemorrhage, ALT increased, and influenza like illness. The moderate TEAE of influenza like illness was considered to be an SAE and was reported by a subject in the SAD part who had received a single dose of 150 mg IMB-1018972 under fasted conditions in the SAD part. The subject was withdrawn from the study as a result of this SAE. The SAE was considered by the Investigator unlikely to be related to the study drug.

In both the SAD part and MAD part, there was no clear dose dependency of the number and incidence of TEAEs. In the FE arm of the SAD part, dosing under fed conditions appeared to attenuate the number and incidence of TEAEs.

The most frequently reported TEAEs during the study were of the system organ class vascular disorders (mainly TEAEs of flushing), general disorders and administration site conditions, nervous system disorders, gastrointestinal disorders, and musculoskeletal and connective tissue disorders.

The majority of the TEAEs reported during the study were considered by the Investigator not to be related to the study drug.

There were no findings of clinical relevance with respect to clinical laboratory, vital signs, 12-lead ECG, continuous cardiac monitoring (telemetry), or physical examination.

### Pharmacokinetics

All blood samples of subjects that received IMB-1018972 in this study were analyzed for IMB-1018972 in plasma, but IMB-1018972 could be measured in only few plasma samples. Therefore, the IMB-1018972 concentrations have only been listed and no descriptive statistics or concentration-time profiles have been presented in this clinical study report. In addition, no PK parameters have been calculated for plasma IMB-1018972. As a result, urine samples were not analyzed for IMB-1018972 concentrations.

Since the pharmacodynamic effect of IMB-1028814 and trimetazidine is the same, data are presented for IMB-1028814 and trimetazidine individually, as well as for the sum of IMB-1028814 and trimetazidine concentrations.

### SAD Part (and Integrated FE Arm)

### PK in Plasma Following Administration of IMB-1018972 Under Fasted Conditions

The initial hydrolysis of IMB-1018972 to IMB-1028814 and subsequent systemic bioavailability of IMB-1028814 was relatively rapid with median tₘₐₓ around 1 hour postdose for IMB-1028814, and between 1.5 hours and 2 hours postdose for trimetazidine over the studied single-dose range of 50 mg to 400 mg IMB-1018972 under fasted conditions. Median tₘₐₓ did not increase with increasing IMB-1018972 dose.

The geometric mean Cₘₐₓ increased with dose and ranged between 104 ng/mL and 870 ng/mL for IMB-1028814, between 36.9 ng/mL and 274 ng/mL for trimetazidine, and between 516 nmol/L and 3,839 nmol/L (molar units to account for differences in molecular weight) for IMB-1028814 + trimetazidine over the studied single-dose range of 50 mg to 400 mg IMB-1018972 under fasted conditions. Similarly, the geometric mean AUC₀₋ₜ increased with dose and ranged between 290 ng.h/mL and 2,795 ng.h/mL for IMB-1028814, between 424 ng.h/mL and 3,305 ng.h/mL for trimetazidine, and between 2,970 nmol.h/L and 22,365 nmol.h/L for IMB-1028814 + trimetazidine over the studied single-dose range of 50 mg to 400 mg IMB-1018972 under fasted conditions. The predefined stopping criterion for IMB-1028814 plasma exposure of 417,733 and 652,849 ng.h/mL for males and females, respectively, was not reached by any of the subjects during the SAD part.

Dose proportionality of IMB-1028814 and trimetazidine was explored for Cₘₐₓ, AUC0-t, and AUC0-inf. The 95% CIs of the slopes of all 3 exposure parameters included 1 for both IMB-1028814 and trimetazidine indicating no evidence of a deviation from dose proportionality of IMB-1028814 and trimetazidine over the IMB-1018972 single-dose range of 50 to 400 mg under fasted conditions.

The geometric mean t_{½} of IMB-1028814 was relatively short, ranging between 2.6 hours and 3 hours over the IMB-1018972 single-dose range under fasted conditions. For metabolite trimetazidine, geometric mean t_{½} was longer, ranging between 6.76 hours and 8 hours over the IMB-1018972 single-dose range under fasted conditions. Geometric mean t_{½} of IMB-1028814 and trimetazidine did not increase with increasing IMB-1018972 dose indicating that the PK of the 2 moieties was linear.

### PK in Plasma Following Administration of Trimetazidine

Following administration of a single oral dose of 35 mg trimetazidine, median trimetazidine tmax was 5 hours, and geometric mean values were 68.6 ng/mL for Cmax, 912 ng.h/mL for AUC₀₋ₜ, and 929 ng.h/mL for AUC_{0-inf}. The geometric mean t1/2 of trimetazidine was 7.49 hours.

### Effect of Food

The possible effect of food on the PK of IMB-1028814 and trimetazidine was explored by comparing administration of single oral doses of 150 mg IMB-1018972 after an FDA-defined high-fat breakfast and under fasted conditions.

Median IMB-1028814 tₘₐₓ in plasma was reached at 2 hours postdose under fed conditions relative to 1 hour postdose under fasted conditions. Median trimetazidine tₘₐₓ in plasma was reached at 4 hours postdose under fed conditions relative to 1.5 hours postdose under fasted conditions.

The effect of food of IMB-1028814 and trimetazidine was explored for Cmax, AUC0-t, and AUC_{0-inf}. No evidence for an effect of food was observed on the IMB-1028814 exposure parameters AUC₀₋ₜ and AUC_{0-inf} (both with an estimate of 1.12 and 90% CI ranging from 1.02 to 1.23). However, Cₘₐₓ was approximately 36% lower following administration of a single dose of 150 mg IMB-1018972 after an FDA-defined high-fat breakfast relative to administration under fasted conditions (estimate of 0.64; 90% CI ranging from 0.39 to 1.04).

No evidence for an effect of food was observed on the trimetazidine exposure parameters Cₘₐₓ (estimate of 0.91; 90% CI ranging from 0.85 to 0.98), and AUC0-t and AUC0-inf (both with an estimate of 1.04 and 90% CI ranging from 0.98 to 1.10) following administration of a single dose of 150 mg IMB-1018972.

### PK in Urine

The arithmetic mean percent of the dose excreted in urine ranged between 3.99% and 5.74% for IMB-1028814, and between 23.11% and 32.55% for trimetazidine within 48 hours after a single oral IMB-1018972 dose over the studied dose range of 50 mg to 400 mg. Within 48 hours following administration of a single oral dose of 35 mg trimetazidine, an arithmetic mean of 54.47% was excreted in urine as trimetazidine. These results indicate that metabolism is the primary clearance mechanism for IMB-1028814 while renal excretion is the primary clearance mechanism for trimetazidine.

The geometric mean renal clearance (CLR) ranged between 3.76 L/h and 5.37 L/h for IMB-1028814, and between 18.1 L/h and 20.8 L/h for trimetazidine over the studied single-dose range of 50 mg to 400 mg IMB-1018972. Geometric mean CLR for trimetazidine was 20.4 L/h following administration of a single oral dose of 35 mg trimetazidine. The renal clearance of trimetazidine is greater than the glomerular filtration rate (125 mL/min or 7.5 L/h), indicating that trimetazidine undergoes net tubular secretion.

### MAD Part

Over the 2 multiple-dose levels, median tₘₐₓ ranged between 0.5 hours and 1 hours postdose for IMB-1028814 on Day 1, and was 3 hours postdose for trimetazidine on Day 1. On Day 14, median tₘₐₓ was 0.5 hours postdose for IMB-1028814 and 2 hours postdose for trimetazidine.

### Exposure Parameters on Day 1

No dose-proportionality analysis was done since there were only 2 IMB-1018972 dose levels in the MAD part: multiple oral doses of 50 mg or 150 mg q12h for 14 days under fed conditions.

The geometric mean Cₘₐₓ and AUC0-_{T} were higher after 150 mg fed than after 50 mg fed for IMB-1028814 (297% and 336% higher for Cₘₐₓ and AUC_{0-T}, respectively), trimetazidine (154% and 163% higher for Cₘₐₓ and AUC_{0-T} respectively), and IMB-1028814 + trimetazidine (257% and 239% higher for Cₘₐₓ and AUC_{0-T}, respectively).

When comparing the MAD and SAD parts, geometric mean Cₘₐₓ was 97% higher on Day 1 after 150 mg fed in the MAD part than after a single dose of 150 mg fed in the SAD part for IMB-1028814. For trimetazidine however, geometric mean Cₘₐₓ was 32% lower on Day 1 after 150 mg fed in the MAD part than after a single dose of 150 mg fed in the SAD part.

### Exposure Parameters on Day 14 Following Repeated Q12h Dosing

The geometric mean Cₘₐₓ and AUC_{0-T} were higher after 150 mg fed than after 50 mg fed for IMB-1028814 (377% and 367% higher for Cₘₐₓ and AUC_{0-T}, respectively), trimetazidine (127% and 126% higher for Cₘₐₓ and AUC_{0-T}, respectively), and IMB-1028814 + trimetazidine (286% and 211% higher for Cmax and AUC_{0-T}, respectively).

The predefined stopping criterion for IMB-1028814 plasma exposure of 417,733 and 652,849 ng.h/mL for males and females, respectively, was not reached by any of the subjects during the MAD part.

### Trough Concentrations Following Repeated Q12h Dosing

Based upon visual inspection of the geometric mean plasma concentration-time profiles and the geometric mean trough concentrations, it can be concluded that for both 150 mg fed and 50 mg fed, the Day 14 IMB-1018972 dose was administered under steady-state conditions of IMB-1028814 and trimetazidine concentrations.

### Accumulation Following Repeated Q12h Dosing

For both the 50 mg and 150 mg fed dose levels, geometric mean AUC_{0-T} values of IMB-1028814, trimetazidine, and IMB-1028814 + trimetazidine were higher on Day 14 relative to Day 1. Geometric mean Rac for IMB-1028814 was 1.18 and 1.10 after the 150 mg fed dose and 50 mg fed dose, respectively, indicating minimal accumulation of IMB-1028814 in plasma. Geometric mean Rac for trimetazidine was 1.63 and 1.89 after the 150 mg fed dose and 50 mg fed dose, respectively, indicating modest accumulation of trimetazidine in plasma. Geometric mean Rac for IMB-1028814 + trimetazidine was 1.39 and 1.52 after the 150 mg fed dose and 50 mg fed dose, respectively, indicating modest accumulation of IMB-1028814 + trimetazidine in plasma.

### Terminal Elimination Half-life Following Repeated Q12h Dosing

For IMB-1028814, the geometric mean t1/2 of 4.48 hours after 150 mg fed was longer than that of 2.79 hours after 50 mg fed. For trimetazidine, the geometric mean t1/2 of 9.36 hours after 150 mg fed was similar to that of 9.32 hours after 50 mg fed. For IMB-1028814 + trimetazidine, the geometric mean t_{½} of 8.90 hours for IMB-1028814 after 150 mg fed was similar to that of 9.08 hours after 50 mg fed.

### Conclusions

### Safety

Overall, single oral IMB-1018972 doses and multiple oral IMB-1018972 doses of an IR formulation were generally well tolerated by healthy male and female subjects. There were no findings of clinical relevance with respect to clinical laboratory, vital signs, 12-lead ECG, continuous cardiac monitoring (telemetry), or physical examination. Of note, there were no findings of hemodynamic changes, nor changes in the QTc-interval, after administration of IMB-1018972 as the IR.
During the SAD part, the most common AEs were 6 TEAEs of flushing (reported terms were ‘niacin flush’ and ‘flushing neck’), of which 5 TEAEs were of moderate severity and 1 TEAE was of mild severity. Four subjects reported flushing after a single dose of 400 mg IMB-1018972 under fasted conditions, and 2 subjects of the FE arm reported flushing after a single dose of 150 mg IMB-1018972 under fasted conditions. These TEAEs were all considered by the Investigator to be related to the study drug. No subjects dropped out due to flushing and flushing was not considered a safety issue. Dose escalation beyond 400 mg IMB-1018972 IR did not proceed as planned based on the PK exposure levels of IMB-1028814 and trimetazidine exceeding the target exposure levels in the 400 mg group and the findings of flushing at that dose. The predefined target exposure level was approximately 3 to 4 ‘trimetazidine equivalents’, ie, the ratio of the combined exposure of the active metabolites of IMB-1018972 to the single oral doses of 35 mg MR trimetazidine as seen in published literature.
There were no deaths reported during the study. Most TEAEs were of mild severity and no severe TEAEs were reported during the study. Overall, 12 of a total of 181 TEAEs were of moderate severity.
Two subjects were withdrawn from the study: 1 subject due to a moderate SAE of influenza like illness (unlikely related) and 1 due to a moderate TEAE of ALT increased (possibly related).
Overall, there was no clear dose dependency of the number and incidence of TEAEs.
Dosing under fed conditions appeared to attenuate the number and incidence of TEAEs in the FE arm of the SAD part.

### Pharmacokinetics

IMB-1018972 could be measured in only few plasma samples taken during this study.
When combining the single and multiple IMB-1018972 dose results under fasted and fed conditions, the initial hydrolysis of IMB-1018972 to IMB-1028814 and subsequent systemic bioavailability of IMB-1028814 was relatively rapid with median tmax ranging between 0.5 hours and 5 hours postdose for IMB-1028814, and between 1.5 hours and 8 hours postdose for trimetazidine. Median tmax did not increase with increasing IMB-1018972 dose
The predefined stopping criterion for IMB-1028814 plasma exposure of 417,733 and 652,849 ng.h/mL for males and females, respectively, was not reached by any of the subjects during the SAD part or MAD part.
Following single oral IMB-1018972 doses in the range of 50 to 400 mg under fasted conditions, systemic exposure to IMB-1028814 and trimetazidine was dose proportional for Cₘₐₓ, AUC₀₋ₜ, and AUC_{0-inf}.
No evidence for an effect of food was observed on the IMB-1028814 exposure parameters AUC₀₋ₜ and AUC_{0-inf} following administration of a single dose of 150 mg IMB-1018972. However, Cₘₐₓ was approximately 36% lower following administration of a single dose of 150 mg IMB-1018972 under fed conditions relative to administration under fasted conditions.
No evidence for an effect of food was observed on the trimetazidine exposure parameters Cₘₐₓ, AUC₀₋ₜ, and AUC_{0-inf} following administration of a single dose of 150 mg IMB-1018972.
When combining the single and multiple IMB-1018972 dose results under fasted and fed conditions, the geometric mean t1/2 ranged between 2.5 hours and 4.5 hours for IMB-1028814, and between 6.5 hours and 9.5 hours for trimetazidine. Geometric mean t_{½} did not increase with increasing IMB-1018972 dose.
Within 48 hours following administration of a single oral dose of IMB-1018972 over the range of 50 mg to 400 mg, on average between 3.99% and 5.74% of the dose was excreted in urine as IMB-1028814, and on average between 23.11% and 32.55% of the dose was excreted as trimetazidine.
Within 48 hours following administration of a single oral dose of 35 mg trimetazidine, on average 54.47% of the dose was excreted in urine as trimetazidine.
Following 14 days of twice daily dosing with 150 mg and 50 mg IMB-1018972 under fed conditions, no relevant accumulation was observed of IMB-1028814 (Rac of 1.18 and 1.10 for 150 mg and 50 mg, respectively) and accumulation of trimetazidine was modest (Rac of 1.63 and 1.89 for 150 mg and 50 mg, respectively).

### Overall

In view of the positive risk/benefit profile and the observed PK characteristics of the IMB-1018972 metabolites IMB-1028814 and trimetazidine in this single-dose and multiple-dose FIH study, further clinical development of IMB-1018972 is warranted.

### Detailed Study Design

### Introduction

IMB-1018972 is an orally administered small molecule that is being developed as a treatment for ischemic cardiovascular disease and the associated abnormal cellular energetics. Potential indications include angina pectoris, heart failure, and peripheral vascular disease. IMB-1018972 is a new chemical entity (NCE) of the drug class partial fatty acid oxidation (pFOX) inhibitors that acts to preserve or enhance energy metabolism in cells exposed to hypoxia or ischemia. Other pFOX inhibitors include ranolazine (Ranexa), perhexiline, and trimetazidine. Glucose oxidation is a more efficient producer of adenosine triphosphate per oxygen molecule consumed compared to fatty acid oxidation.

IMB-1018972 undergoes hydrolysis after administration, and the hydrolysis products are nicotinic acid (also known as niacin or vitamin B3) and an inhibitor of 3-ketoacyl CoA thiolase (3-KAT) named IMB-1028814. In addition to IMB-1018972, IMB-1028814 has been studied and characterized extensively in nonclinical studies. IMB-1028814 undergoes further metabolism and 1 metabolite is trimetazidine, a drug marketed in Europe since 1987 for the treatment of angina pectoris.

The primary mechanism of action of IMB-1028814 is thought to be competitive inhibition of 3-KAT that results in the shift of substrate utilization in the myocardium from fatty acid oxidation to glucose oxidation. The delivery of nicotinic acid may serve to additionally enhance cellular energetics.

The nonclinical pharmacology and toxicology data collected at the time the CSP was finalized supported conducting clinical studies that administer IMB-1018972 for up to 4 weeks to assess its safety, tolerability, PK, and pharmacodynamics in humans.

Trimetazidine administered in this study is a drug marketed in Europe since 1978 for the treatment of angina pectoris.

### Study Rationale

No clinical studies with IMB-1018972 had been performed prior to the study described in this CSR. Therefore, this first-in-human study (FIH), with single-dose and multiple-dose escalation designs (single ascending dose [SAD] part and multiple ascending dose [MAD] part) and a single-dose food effect (FE) study was conducted to assess the safety, tolerability, and PK of IMB-1018972 as an immediate-release (IR) formulation following administration of single and multiple ascending doses.

During the study, a group was added to the SAD part testing a single 35-mg modified-release (MR) dose of trimetazidine (Vastarel). The primary rationale for adding this group was to study the PK profile of commercially available trimetazidine with the same analytical assays utilized in the current study, which would enable a direct comparison of the PK profiles of trimetazidine generated from Vastarel and that generated from the metabolism of IMB-1028814. The analytical assays include detection of trimetazidine in blood and urine, which is the primary route of elimination. These data, together with the data generated from the MAD part would help the Sponsor select doses for further investigation in the Phase 2 proof-of-concept study in patients with refractory angina.

### Study Objectives

### Primary

To assess the safety and tolerability of single and multiple ascending oral doses of IMB-1018972, single oral doses of trimetazidine.

### Secondary

To assess the PK profile of single and multiple ascending oral doses of IMB-1018972, single oral doses of trimetazidine.
To assess the effect of food on the absorption and the PK profile of IMB-1018972 following a single oral dose of IMB-1018972 in healthy subjects
To evaluate the effect of food on the safety and tolerability of IMB-1018972 following a single oral dose of IMB-1018972 in healthy subjects

### Investigational Plan

### Overall Study Design and Plan

### Type of Study

This was a double-blind, randomized, placebo-controlled study, consisting of a SAD part with integrated FE arm, a MAD part to assess the safety, tolerability, and PK of ascending single and multiple oral doses of IMB-1018972 (IR formulation in the SAD and MAD parts), and single oral doses of a MR formulation of trimetazidine. The study started with the SAD part.

### SAD Part (and Integrated FE Arm)

In the SAD part, 5 groups of 8 healthy subjects (6 subjects on active drug and 2 on placebo in Groups A1, A2, A3, and A4, and 8 subjects on active drug in Group A5) were included. In Groups A1, A2, A3, and A4, subjects received a single oral dose of an IR formulation of IMB-1018972 or placebo under fasted conditions (an overnight fast of at least 10 hours). In Group A5, all subjects received a single oral dose of a MR formulation of trimetazidine under fasted conditions (an overnight fast of at least 10 hours). Each subject participated in only 1 group during the study

Subjects assigned to Group A4 also participated in the FE arm and received the same single dose of IMB-1018972 or placebo under fed conditions (Food and Drug Administration [FDA]-defined high-fat breakfast after an overnight fast of at least 10 hours) in a second period at least 1 week after drug administration under fasted conditions in the SAD part.

In this first-in-human (FIH) study, the subjects participating at the lowest dose level, subjects of Group A1, were dosed according to a sentinel dosing design to ensure optimal safety. This means that initially, 2 subjects were dosed: 1 subject with IMB-1018972 and 1 subject with placebo. Since the safety and tolerability results of the first 24 hours following dosing for the initial 2 subjects were acceptable to the Investigator, the other 6 subjects (5 active drug and 1 placebo) of the lowest dose level were also dosed. Depending on emerging safety data, it could have been decided to implement this sentinel dosing design for other groups as well; however, this was not done.

The SAD part consisted of:
An eligibility screening period of up to 35 days
One study period involving administration of a single dose of IMB-1018972 or placebo (or trimetazidine in Group A5); this was 2 periods for subjects of Group A4 also participating in the FE arm
Safety assessments and blood sampling for PK purposes from predose up to 48 hours after drug administration
Discharge at 48 hours after study drug administration (in each period for subjects of Group A4 also participating in the FE arm)
A follow-up visit 7 to 14 days after the last PK blood sample; this was 7 to 14 days after the last PK blood sample in the second period for subjects of Group A4 also participating in the FE arm

### MAD Part

In the MAD part, 2 groups of 12 healthy subjects (9 subjects on active drug and 3 on placebo in each group) were included. Subjects received multiple oral doses of an IR formulation of IMB-1018972 or placebo once q12h for 14 consecutive days. Each subject participated in only 1 group during the study.

Study drug administration was under fed conditions as determined based on the results of Group A4 in the FE arm.

The MAD part consisted of:
An eligibility screening period of up to 35 days
One study period involving administration of multiple doses of IMB-1018972 or placebo for 14 consecutive days
Safety assessments and blood sampling for PK purposes from predose up to 48 hours after the last study drug administration
Discharge at 48 hours after the last study drug administration
A follow-up visit 7 to 14 days after the last PK blood sample

### Screen Period

Subjects reported to the medical screening facility for the eligibility screening within 5 weeks prior to (the first) study drug administration.

Subjects signed the study-specific ICF prior to any study-specific screening procedures being performed. The written informed consent was obtained for all subjects, regardless of their eligibility for the study. The signed ICFs were retained and archived at PRA and a copy was provided to the subject.

### Treatment Period

Subjects were in the clinic for 1 treatment period (2 treatment periods for subjects of Group A4 also participating in the FE arm). The subjects were admitted to the clinical research center in the afternoon of Day -1. Day 1 was the day of (the first) drug administration.

Subjects of the SAD part were discharged on Day 3 (48 hours after study drug administration) after completion of the assessments; discharge was on Day 3 of each period for subjects of Group A4 also participating in the FE arm. Subjects of the MAD part were discharged on Day 16 (48 hours after the last study drug administration on Day 14) after completion of the assessments.

### Follow-Up

For the SAD part, the follow-up assessments were performed 7 to 14 days after the last PK blood sample (between Day 10 and Day 17). For the FE arm, the follow-up assessments were performed 7 to 14 days after the last PK blood sample in the second period (between Day 10 and Day 17). For the MAD part, the follow-up assessments were performed 7 to 14 days after the last PK blood sample (between Day 23 and Day 30).

### Discussion of Study Design

### Dose Escalation Within a Study Part

An escalating-dose study design was chosen for the SAD and MAD parts to allow careful increase of the IMB-1018972 dose after assessment of the available safety, tolerability, and PK results of the preceding group.

A dose-escalation meeting was held between the Investigator and the Sponsor. Further, a dose-escalation report (DER) was provided by the Investigator to the IEC following completion of each dose level. Escalation to the next higher dose only proceeded when none of the stopping criteria had been reached and if the available safety, tolerability, and PK results of the preceding group were acceptable to the Investigator and the Sponsor and after a statement of no objection of the DER from the IEC. The safety, tolerability, and PK results had to be available up to 48 hours postdose for the SAD part and up to 48 hours after the final morning dose on Day 14 for the MAD part. In addition, these results had to be available from at least 5 dosed subjects of the preceding group in the SAD part and at least 8 dosed subjects of the preceding group in the MAD part

The planned dose levels to be administered could be changed based on the safety, tolerability, and plasma PK results of the previous group(s).

Dose levels in the MAD part could not exceed dose levels that were well tolerated in the SAD part.

The increase from one dose level to the next dose level could not be more than 3-fold.

Although this was an ascending dose study, if safety or tolerability issues were experienced, a lower dose could be administered in the next groups. Also, the same dose could be tested in 2 groups or an intermediate dose could by tested to gain more information on safety, tolerability, and/or PK.

### Stopping Rules for Dose Escalation

Dosing within a group and dose escalation to a next group was halted at any time if 1 of the following circumstances occurred:
A drug-related serious adverse reaction (ie, a serious adverse event [SAE] considered at least possibly related to the study drug administration) in 1 subject.
Drug-related severe adverse reactions (ie, severe adverse events [AEs] considered at least possibly related to the study drug administration) in 2 subjects in the same group, or in 1 subject in the sentinel group of a group.
Other findings that, at the discretion of the Investigator and/or Sponsor’s Medical Monitor, indicated that further dosing had to be stopped.

When stopping rules for a group were met, the randomization code for subjects meeting the stopping rules was to be unblinded. If after unblinding it was concluded that subjects on active medication met the stopping rules, dosing in the group was to be stopped, and no further dose escalation was to be performed. If a subsequent integrated analysis of available data led to the conclusion that further careful escalation was warranted, a substantial amendment was needed before continuation of the study. Dose escalation in a study part (SAD, MAD) was permanently stopped if:
Blinded PK data indicated that after dose escalation it was anticipated that individual subjects would exceed the predefined maximum exposure level of AUC_{0-8 x 2} for IMB-1028814 of 417,733 and 652,849 ng.h/mL for males and females, respectively

### Sentinel Dosing

IMB-1018972 is in the early stage of clinical development, with the SAD part of the study being the first time the compound was administered to man. In this FIH study, the subjects participating at the lowest dose level of the SAD part, subjects of Group A1, were dosed according to a sentinel dosing design to ensure optimal safety. This means that initially, 2 subjects were dosed. One of these subjects received the active medication IMB-1018972, and the other subject received placebo. The subjects were closely observed by the Investigator for the first 24 hours following drug administration. The general tolerability of the study drug was monitored during this time, and the electrocardiogram (ECG) and vital signs recordings were reviewed. Any reported AEs were also considered in the Investigator’s evaluation. If the safety and tolerability results of the first 24 hours following dosing for the initial 2 subjects were acceptable to the Investigator, the other subjects of the lowest dose level could be dosed in a placebo-controlled randomized manner (5 active and 1 placebo). Depending on emerging safety data, it could have been decided to implement this sentinel dosing design for other SAD groups as well (except for the second period of Group A4 in the FE arm and except for Group A5; all subjects of these 2 groups could be dosed on the same day).

### Effect of Food

Subjects from Group A4 of the SAD part were assigned to the integrated FE arm. After administration of the drug to fasting subjects in the SAD part, the FE arm used the same subjects and experimental procedures. An exception was that subjects consumed an FDA-defined high-fat breakfast prior to dosing to evaluate the possible effect of food on the PK of IMB-1018972. This allowed for a within-subject comparison of the PK of IMB-1018972 in plasma and tolerability after administration in fasted and fed conditions.

### Continuation to the MAD Part of the Study

The MAD part could start after the results from the FE arm were available. The first group of the MAD part could start when a DER, summarizing safety and available PK data of previous SAD groups, concluded that a single dose with an exposure at/above the expected steady-state exposure in the first MAD group was well tolerated.

In the MAD part, subjects received twice daily dosing, which was the anticipated clinical dosing regimen considering the anticipated short human half-life and absence of prolonged duration of action. Doses were given q12h. In the MAD part, dosing continued for 14 days, which was anticipated to result in steady state of exposure.

The highest multiple-dose group planned could not exceed the highest planned single dose of 1600 mg/day or the highest tolerated dose in the SAD part. This was predicted to sufficiently cover doses in future dose-finding studies in patients.

### Other

The planned confinement period, day of discharge, and follow-up period could be adapted depending on emerging study results. Also, the timing, type, and number of safety and PK assessments could be changed during the study.

The purpose of including placebo-treated subjects in each group (except Group A5 in which no placebo was administered) was to assist the medical assessment of whether or not any abnormalities observed were due to the study drug or to study procedures, and not for a formal statistical comparison between active and placebo subjects.

There was no indication from in vitro studies (cytochrome P450 [CYP]3A4/GT1A1/CYP2C19/CYP2C9) for interaction with oral contraceptives. Women of childbearing potential who were using adequate contraception were included in the present study, in order to make the outcome of this FIH study relevant for the female target patient population.

The use of healthy subjects as opposed to patients allowed a clearer interpretation of the study results, as there were no confounding factors resulting from changes in disease state and/or concomitant medications.

The study was performed in different groups of subjects since the number of doses to be tested, and all assessments associated with these sessions, were regarded as too extensive to be performed in a single group of subjects participating repeatedly.

The Investigator took all the usual precautions necessary for studies at an early stage in the development of a new drug.

### Selection of Study Population

The overall study population consisted of 88 subjects.

In the SAD part (and integrated FE part), a total of 40 healthy male or female subjects were included. Eight subjects from Group A4 in the SAD part participated in the FE arm. From Group A4 onwards, all efforts were made to have a ratio of 50:50 for male and female subjects per group, but at minimum at least 3 subjects of each gender were dosed per group.

In the MAD part, a total of 24 healthy male or female subjects were included. For each group, all efforts were made to have a ratio of 50:50 for male and female subjects, but at minimum at least 4 subjects of each gender were dosed per group.

All efforts were made to have a ratio of 50:50 for male and female subjects, but at minimum at least 4 subjects of each gender were dosed in each part.

### Inclusion Criteria

Subjects were eligible for inclusion in the study if they met all the following inclusion criteria:
1. Gender: male or female.
2. Age: 18 years to 65 years, inclusive, at screening.
3. Body mass index (BMI): 18.0 kg/m2 to 32.0 kg/m2, inclusive.
4. Status: healthy subjects.
5. At screening, females could be of childbearing potential (but not pregnant or lactating), or of nonchildbearing potential (either surgically sterilized or physiologically incapable of becoming pregnant, or at least 1 year postmenopausal [amenorrhea duration of 12 consecutive months]); nonpregnancy was confirmed for all females by a serum pregnancy test conducted at screening and each admission.
6. Female subjects of childbearing potential who had a fertile male sexual partner had to agree to use adequate contraception from screening until 90 days after the follow-up visit. Adequate contraception was defined as using hormonal contraceptives or an intrauterine device combined with at least 1 of the following forms of contraception: a diaphragm, a cervical cap, or a condom. Total abstinence, in accordance with the lifestyle of the subject, was also acceptable.
7. Male subjects, if not surgically sterilized, had to agree to use adequate contraception and not donate sperm from (first) admission to the clinical research center until 90 days after the follow-up visit. Adequate contraception for the male subject (and his female partner) was defined as using hormonal contraceptives or an intrauterine device combined with at least 1 of the following forms of contraception: a diaphragm, a cervical cap, or a condom. Total abstinence, in accordance with the lifestyle of the subject, was also acceptable.
8. All prescribed medication had to be stopped at least 30 days prior to (first) admission to the clinical research center. An exception was made for hormonal contraceptives, which could be used throughout the study.
9. All over-the-counter medication, vitamin preparations and other food supplements, or herbal medications (eg, St. John’s Wort) had to be stopped at least 14 days prior to (first) admission to the clinical research center. An exception was made for paracetamol, which was allowed up to admission to the clinical research center.
10. Willingness to abstain from alcohol, methylxanthine-containing beverages or food (coffee, tea, cola, chocolate, energy drinks), grapefruit (juice), and tobacco products from 48 hours prior to (each) admission to the clinical research center.
11. Good physical and mental health on the basis of medical history, physical examination, clinical laboratory, and vital signs, as judged by the Investigator.
12. Had no clinically significant abnormal 12-lead ECG (incomplete right bundle branch block could be accepted) at screening: PR-interval <210 ms, QRS-duration <120 ms, and QTc-interval (Fridericia’s) ≤450 msec for males and females.
13. Willing and able to sign the ICF.

### Exclusion Criteria

Subjects were excluded from participation if any of the following exclusion criteria applied:
1. Previous participation in the current study.
2. Employee of PRA or the Sponsor.
3. History of relevant drug and/or food allergies.
4. Using tobacco products within 3 months prior to (the first) drug administration.
5. History of alcohol abuse or drug addiction (including soft drugs like cannabis products).
6. Positive drug and alcohol screen (opiates, methadone, cocaine, amphetamines [including ecstasy], cannabinoids, barbiturates, benzodiazepines, tricyclic antidepressants, and alcohol) at screening and (each) admission to the clinical research center.
7. Average intake of more than 24 units of alcohol per week (1 unit of alcohol equals approximately 250 mL of beer, 100 mL of wine, or 35 mL of spirits).
8. Positive screen for hepatitis B surface antigen (HBsAg), anti-hepatitis C virus (HCV) antibodies, or anti-HIV 1 and 2 antibodies.
9. Participation in a drug study within 60 days prior to (the first) drug administration in the current study. Participation in more than 4 other drug studies in the 12 months prior to (the first) drug administration in the current study.
10. Donation or loss of more than 100 mL of blood within 60 days prior to (the first) drug administration. Donation or loss of more than 1.5 liters of blood (for male subjects)/more than 1.0 liters of blood (for female subjects) in the 10 months prior to (the first) drug administration in the current study.
11. Significant and/or acute illness within 5 days prior to (the first) drug administration that could impact safety assessments, in the opinion of the Investigator.
12. Unsuitable veins for infusion or blood sampling.
13. For FE Group A4 and the single-dose MR part only: Unwillingness to consume the FDA breakfast.

Please note that subjects were to refrain from consumption of any foods containing poppy seeds within 48 hours (2 days) prior to screening to the clinical research center to avoid false positive drug screen results. In addition, they were to refrain from strenuous exercise within 96 hours (4 days) prior to screening as this could result in abnormal clinical laboratory values.

### Removal of Subject From Assessment

Participation in the study was strictly voluntary. A subject had the right to withdraw from the study at any time for any reason.

The Investigator had the right to terminate participation of a subject for any of the following reasons: difficulties in obtaining blood samples, violation of the protocol, severe AEs or SAEs, or for any other reason relating to the subject’s safety or the integrity of the study data.

If a subject was withdrawn from the study, the Sponsor was to be informed immediately. If there was a medical reason for withdrawal, the subject remained under the supervision of the Investigator until satisfactory health had returned.

Subjects who dropped out or withdrew for any reason without completing all screening evaluations successfully, were considered screening failures.

A subject who was withdrawn or voluntarily withdrew from the study for any reason, whether related to the study drug or not, after having received a subject number, was considered an early-termination subject. If a subject was withdrawn for a reason related to the study drug, according to the judgment of the Investigator, the early-termination subject was not replaced. If a subject did not complete the study for a reason not related to the study drug, the early-termination subject could be replaced after mutual agreement between the Sponsor and PRA.

The decision regarding the replacement of subjects was documented.

PRA made every effort to ensure that early-termination subjects who had received study drug completed the safety follow-up assessments.

### Stopping Rules for Individual Subjects

Dosing of a subject was stopped at any time during the study if any of the following circumstances occurred:
A serious adverse reaction (ie, an SAE considered at least possibly related to the study drug administration).
An overall pattern of clinically significant changes in any safety parameter (eg, moderate or severe AEs in >1 subject) that could appear to be minor in terms of an individual event but, in the opinion of the Sponsor or Investigator, collectively represented a safety concern.
Other findings that, at the discretion of the Investigator and/or Sponsor’s Medical Monitor, indicated that further dosing should be stopped.

### Treatments

### SAD Part (and Integrated FE Arm)

The following treatments were administered under fasted conditions according to the randomization code:
Group A1 single oral dose of 50 mg IR formulation of IMB-1018972 (n=6) or matching placebo (n=2) on Day 1
Group A2 single oral dose of 150 mg IR formulation of IMB-1018972 (n=6) or matching placebo (n=2) on Day 1
Group A3: single oral dose of 400 mg IR formulation of IMB-1018972 (n=6) or matching placebo (n=2) on Day 1
Group A4: single oral dose of 150 mg IR formulation of IMB-1018972 (n=6) or matching placebo (n=2) on Day 1 (FE group)
Group A5: single oral dose of 35 mg MR formulation of trimetazidine (Vastarel; n=8) on Day 1
The following treatment was administered in the FE arm under fed conditions (FDA-defined high-fat breakfast) according to the randomization code
Group A4: single oral dose of 150 mg IR formulation of IMB-1018972 (n=5) or matching placebo (n=2) on Day 1 (same dose as in SAD part)
Up to 2 additional SAD groups could be included to evaluate a lower, intermediate, or repeat dose level(s), or, provided that dose-escalation termination criteria had not been met, a higher dose level.

### MAD Part

The following treatments were administered according to the randomization code under fed conditions as determined based on the results of Group A4 in the FE arm. The doses were selected based upon the safety, tolerability, and PK data from the SAD part:
Group B1: multiple oral doses of an IR formulation of 150 mg IMB-1018972 (n=9) or matching placebo (n=3) twice daily (q12h) for 14 days; on Day 14 only a single morning dose was administered
Group B2: multiple oral doses of an IR formulation of 50 mg IMB-1018972 (n=9) or matching placebo (n=3) q12h for 14 days; on Day 14 only a single morning dose was administered
Up to 2 additional MAD groups could be included to evaluate a lower, intermediate, or repeat dose level(s), or, provided that dose-escalation termination criteria had not been met, a higher dose level.

### Identity of Investigational Products

### Active Medication

Drug product: IMB-1018972
Activity: Fatty acid oxidation inhibitor
In development for: Ischemic cardiovascular disease
Strength: 25 mg, 100 mg, and 200 mg IR formulations (based on free base)
Dosage form: Oral IR capsule(s) to be used in the SAD and MAD parts
Manufacturer: Pharmacy at PRA
Batch number: 2479-1810-00441 (drug substance)

### IMB-1018972 Placebo (Visually Matching Active Medication)

Active substance: Not applicable
Activity: Not applicable
Strength: Not applicable
Dosage form: Oral capsule(s)
Manufacturer: Pharmacy at PRA
Batch number: Not applicable

### Active Medication

Drug product: Vastarel MR (trimetazidine dihydrochloride)
Activity: Fatty acid oxidation inhibitor
In development for: Angina pectoris
Strength: 35 mg
Dosage form: Oral MR tablet
Manufacturer: Servier Research & Pharmaceuticals (Pakistan) (Pvt.) Ltd.
Batch number: 273782 (drug product)

The study drug was stored in the pharmacy at PRA in a locked facility under the required storage conditions with continuous monitoring. The study drug was dispensed by the pharmacist to the Investigator or authorized designee.

The total number of IMB-1018972 capsules given per dose level in the SAD part (and integrated FE arm) and MAD part is given in Table 1. The number of placebo capsules that was administered to a placebo subject in a specific group was the same as the number of IMB-1018972 capsules that was given to an IMB-1018972 subject in that group.

TABLE 7

Number of IMB-1018972 Capsules Given per Dose Level in the SAD Part (and Integrated FE Arm) and MAD Part

| IMB-1018972 dose level | Number of 25-mg (free base) IMB-1018972 capsules | Number of 100-mg (free base) IMB-1018972 capsules | Number of 200-mg (free base) IMB-1018972 capsules | Total number of IMB-1018972 capsules |
|---|---|---|---|---|
| 50 | 2 | 0 | 0 | 2 |
| 150 | 2 | 1 | 0 | 3 |
| 400 | 0 | 0 | 2 | 2 |

### Method of Assignment Subjects to Treatment Groups

After obtaining informed consent, subjects were screened according to the inclusion and exclusion criteria. Subjects who met all eligibility criteria received a subject number upon inclusion in the study. They received the subject number just prior to dosing according to the randomization code generated by the Biostatistics Department of PRA. The subject number ensured identification throughout the study.

Subject numbers were 101 to 140 for the SAD part, 201 to 224 for the MAD part. Any additional subjects to be included in the SAD part were to be numbered starting from subject number 141 and any additional subjects in the MAD part were to be numbered starting from subject number 225.

Any replacement subject was to receive the number of the subject to be replaced, increased by 200, and was to be administered the same treatment(s). Subjects were assigned to a study part and group based on their availability. Treatments within a group were assigned according to the randomization code generated by the Biostatistics Department of PRA.

In each SAD group, except for Group A5, 6 subjects were randomly assigned to receive IMB-1018972 and 2 subjects were randomly assigned to receive placebo. In Group A5, all 8 subjects received trimetazidine. In each MAD group, 9 subjects were randomly assigned to receive IMB-1018972 and 3 subjects were randomly assigned to receive placebo.

For the 2 sentinel subjects in Group A1 of the SAD part, randomization ensured that 1 subject received IMB-1018972 and the other subject received placebo. For the remaining 6 subjects of Group A1, randomization ensured that 5 received IMB-1018972 and 1 received placebo. Depending on emerging safety data, it could be decided to implement this sentinel dosing design for other SAD groups as well (except for the second period of Group A4 in the FE arm and except for Group A5; all subjects of these 2 groups could be dosed on the same day).

Subjects who dropped out or withdrew for any reason without completing all screening evaluations successfully were considered screening failures. Such subjects, and also subjects who were eligible for inclusion in the study but did not receive the study drug, received no subject number, and only applicable data were entered in the eCRFs.

### Selection of Doses in the Study

Based on the nonclinical toxicology data, it was considered that subjects in this clinical study were not at unreasonable risk of adverse effects. Based on the 28-day dog no observed adverse effect level (NOAEL) of 200 mg/kg/day (oral), the calculated human equivalent dose (HED) is 108 mg/kg/day. For a 60-kg individual, the NOAEL dose would be 6480 mg. With a 10-fold safety factor applied, this would allow for a maximum recommended starting dose (MRSD) of 648 mg/day.7,8 The planned starting dose in the current Phase 1 study was 50 mg, equivalent to 0.83 mg/kg/day for a 60-kg subject. This starting dose is less than 10% of the MRSD determined from the dog NOAEL and less than 1% of the dog NOAEL.

The maximum planned dose in this study of 1600 mg in healthy volunteers was 25% of the HED NOAEL dose of 6480 mg and only 2.5 fold higher than the MRSD. The conservative dosing margin was expected to cover potential supratherapeutic exposures, for instance in patients with renal or hepatic impairment, or in case of potential drug interactions with IMB-1018972. This risk for healthy volunteers at these exposure levels was determined to be acceptable based on the absence of irreversible or significant toxicities without sentinel safety biomarkers.

The relevant animal study was the 28-day dog study where the NOAEL for IMB-1018972 was 200 mg/kg/day. The AUC_{0-8×2} for IMB-1028814 on Day 26 at this dose was 417,733 and 652,849 ng.h/mL for males and females, respectively. The AUC₀₋₈_{×2} for trimetazidine on Day 26 at this dose was 15,042, and 13,834 ng.h/mL for males and females, respectively.

A cohort was added by the Sponsor that was testing a single 35 mg MR dose of trimetazidine (Vastarel). This dose was selected as it is the most commonly used dose of trimetazidine in treating angina and it was therefore known that it has an efficacious PK profile.

### Timing of Doses in the Study

The study drug was administered with 240 mL of tap water to the subject in the upright position. If needed, an additional volume of water was allowed to consume the capsules/tablets comfortably; this additional volume was documented in the eCRF. The dose was given between 08:00 h and 11:00 h, and between 20:00 h and 23:00 h for the afternoon/evening dose. Dosing for each individual subject was at around the same time (±15 min) on each dosing day. The study drug was not chewed.

Administration of the study drug was supervised by the Investigator or authorized designee. After drug administration, a mouth and hand inspection took place.

### Dosing Under Fasted Conditions

### SAD Part 9 and Integrated FE Arm)

Before dosing, subjects fasted overnight for at least 10 hours following a light supper on the evening before. Following dosing, subjects fasted for 4 hours until lunch. During fasting, fluids other than water were not allowed; however, water was not allowed from 2 hours predose until 1 hour postdose (apart from the water taken with the dose).

Subjects of Group A4, also participating in the FE arm, were not allowed to lie down for 4 hours after dosing, except when required for assessments that needed to be performed.

### Dosing Under Fed Conditions

### FE Arm

Before dosing, subjects fasted overnight for at least 10 hours following a snack on the evening before. Then, subjects received an FDA-defined high-fat breakfast that had to be consumed within 20 minutes. The entire breakfast had to be consumed by the subjects. Dosing occurred at 30 minutes after the start of breakfast. Following dosing, subjects fasted for 4 hours until lunch. During fasting, fluids other than water were not allowed.

Subjects of Group A4 also participating in the FE arm were not allowed to lie down for 4 hours after dosing, except when required for assessments that needed to be performed.

### MAD Arm

### Morning Dose

Before each morning dose, subjects fasted overnight for at least 10 hours following a snack on the evening before. On Days 1 and 14, subjects received a standardized breakfast that had to be consumed within 20 minutes. Dosing occurred at 30 minutes after the start of breakfast. Following dosing, subjects fasted for 4 hours until lunch. During fasting, fluids other than water were not allowed. On Days 2 to 13, breakfast was not standardized and was given within maximally 1 hour before dosing and consumed before dosing. No fasting after dosing was applicable on these days.

### Evening Dose

On all dosing days, an evening snack was given within maximally 1 hour before dosing and consumed before dosing.

### Meals During the Study

A fasting period of at least 4 hours was required before obtaining clinical laboratory samples at all time points.

When not fasting, meals and snacks (such as decaffeinated coffee, herbal tea, fruit, and biscuits) were provided according to PRA standard operating procedures (SOPs). A light supper was provided on the evening before those days where fasting was required until lunch time (fasted conditions); a snack was provided on the evening before those days where fasting was required until the FDA-defined high-fat breakfast or breakfast (fed conditions).

For the second period of Group A4 in the FE arm, the FDA-defined high-fat breakfast of 918 kcal consisted of:
2 fried eggs (in 15 g butter/margarine) (approximately 100 g)
1 portion of bacon (40 g) (or brie 60+ for vegetarians)
1 portion of fried potatoes (115 g)
2 slices of (toasted) (wheat) bread (approximately 70 g) with 15 g margarine
1 glass of whole milk (240 mL)

The total of 918 kcal (vegetarian version 915 kcal) could be broken down as follows:
39 g protein = 156 kcal
59 g fat = 527 kcal
59 g carbohydrates = 235 kcal

### Blinding

In each group of the SAD part, except for Group A5, 6 subjects received IMB-1018972 and 2 subjects received placebo according to the randomization code. In Group A5, all 8 subjects received trimetazidine. In each group of the MAD part, 9 subjects received IMB-1018972, and 3 subjects received placebo according to the randomization code. The following controls were employed to maintain the double-blind status of the study:
The oral capsules containing active drug or placebo were indistinguishable in
appearance and taste.
The randomization code was provided to the pharmacist at PRA for dispensing
purposes and kept in the pharmacy, accessible to the pharmacist and the pharmacy
assistant only.

Individual code break envelopes were provided for all subjects by PRA. Each sealed envelope containing the randomization code was kept in a medication storage room that was locked with restricted access. To manage the subject’s condition in case of a medical emergency, the Investigator was allowed to break the code to know whether a subject received IMB-1018972 or placebo. If opened, the name of the person who opened it, the date and time of opening, and the reason for opening were to be written on the envelope. The Sponsor was to be informed in case of unblinding.

The Bioanalytical Laboratory of PRA where the PK samples were analyzed was provided a copy of the randomization code by the pharmacy since only samples of subjects who had received the active drug IMB-1018972 were to be analyzed.

### Previous and Concomitant Therapy and Other Restrictions During the Study

The use of all prescribed medication was not allowed from (first) admission to the clinical research center until follow-up. An exception was made for hormonal contraceptives, which were allowed throughout the study. The use of all over-the-counter medication, vitamin preparations and other food supplements, or herbal medications (eg, St. John’s Wort) was not allowed from (first) admission to the clinical research center until follow-up. An exception was made for paracetamol: from (first) admission onwards, the Investigator could permit a limited amount of paracetamol for the treatment of headache or any other pain. Other medication to treat AEs could only be prescribed if deemed necessary by the Investigator. If medication was used, the name of the drug, the dose, and dosage regimen were recorded in the eCRF.

The use of alcohol, methylxanthine-containing beverages or food (coffee, tea, cola, chocolate, energy drinks), grapefruit (juice), and tobacco products was not allowed during the stay in the clinical research center.

Strenuous exercise was not allowed within 96 hours (4 days) prior to (each) admission and during the stay(s) in the clinical research center.

Subjects were not allowed to consume any foods containing poppy seeds within 48 hours (2 days) prior to (each) admission to the clinical research center as this could cause a false positive drug screen result.

Female subjects of childbearing potential, with a fertile male sexual partner, were required to use adequate contraception (see description below) from screening until 90 days after the follow-up visit.

Male subjects, if not surgically sterilized, were required to use adequate contraception (see description below) and not donate sperm from (first) admission to the clinical research center until 90 days after the follow-up visit.

Adequate contraception was defined as using hormonal contraceptives or an intrauterine device combined with at least 1 of the following forms of contraception: a diaphragm, a cervical cap, or a condom. Total abstinence, in accordance with the lifestyle of the subject, was also acceptable.

Subjects were not allowed to donate blood during the study until the follow-up visit (other than the blood sampling planned for this study).

### Treatment Compliance

Study drug was administered in the clinical research center. To ensure treatment compliance, administration of the study drug was supervised by the Investigator or authorized designee. Compliance was further confirmed by bioanalytical assessment of IMB-1018972, IMB-1028814, and trimetazidine in plasma and urine samples.

The exact times of study drug administration and the number of units administered were recorded in the eCRF. Drug accountability procedures as specified in the CSP were followed.

### Safety and Pharmacokinetic Measurements and Variables

The present study was performed to assess safety, tolerability, and PK following single and multiple doses of single and multiple oral doses of IMB-1018972, single oral doses of trimetazidine. This study did not comprise efficacy or pharmacodynamic assessments.

### Adverse Events

AEs were recorded from (first) admission until completion of the follow-up visit. Any clinically significant observations in results of clinical laboratory, 12-lead ECGs, vital signs, or physical examinations were recorded as AEs.

A treatment-emergent AE (TEAE) was defined as any event not present prior to (the first) administration of the study drug or any event already present that worsened in either severity or frequency following exposure to the study drug.

An AE that occurred prior to (the first) administration of the study drug was considered a pretreatment AE.

At several time points before and after drug administration, subjects were asked nonleading questions to determine the occurrence of AEs. Subjects were asked in general terms about any AEs at regular intervals during the study. In addition, all AEs reported spontaneously during the course of the study were recorded. Details included description of the event, date and time of onset, date and time of end, total duration, severity, relationship to study drug, intervention, seriousness, and outcome. All answers were interpreted by the Investigator and were recorded in the eCRF. All AEs were classified according to the Medical Dictionary for Regulatory Activities (MedDRA; Version 22.0) for AEs.

The severity of the AEs was rated as mild, moderate, or severe; the relationship between the AEs and the study drug was indicated as none, unlikely, possibly, likely, or definitely. Adverse events assessed as possibly, likely, or definitely were considered related to the study drug; AEs assessed as none or unlikely were considered not related to the study drug.

Concomitant medication or other therapy required in case of any AEs was recorded. Concomitant medications were classified according to the World Health Organization Drug Dictionary (Version 22.0).

All AEs were followed up until their resolution or stabilization.

### Clinical Laboratory

Blood and urine samples for clinical laboratory assessments were collected according to PRA SOPs.

The following parameters were measured:
Clinical chemistry (serum quantitatively): total bilirubin, alkaline phosphatase, gamma glutamyl transferase, aspartate aminotransferase (AST), alanine aminotransferase (ALT), lactate dehydrogenase, creatinine, urea, total protein, glucose, inorganic phosphate, sodium, potassium,
calcium, and chloride
Hematology (blood quantitatively): leukocytes, erythrocytes, hemoglobin, hematocrit, thrombocytes, partial automated differentiation (lymphocytes, monocytes, eosinophils, basophils, and neutrophils), mean corpuscular volume, mean corpuscular hemoglobin, and mean corpuscular hemoglobin concentration
Coagulation (blood quantitatively): prothrombin time (reported in seconds and as international normalized ratio), activated partial thromboplastin time, and fibrinogen
Urinalysis (urine qualitatively): hemoglobin, urobilinogen, ketones, glucose, and protein
Serology: HBsAg, anti-HCV, and anti-HIV 1 and 2
Drug and alcohol screen: opiates, methadone, cocaine, amphetamines (including ecstasy), cannabinoids, barbiturates, benzodiazepines, tricyclic antidepressants, and alcohol
Pregnancy test (females only): β-human chorionic gonadotropin in serum

Urine for urinalysis was taken from the PK urine collection container at the end of a collection interval.

In case of unexplained or unexpected clinical laboratory test values, the tests were repeated as soon as possible and followed up until the results had returned to the normal range and/or an adequate explanation for the abnormality was found. The clinical laboratory clearly marked all laboratory test values that were outside the normal range, and the Investigator indicated which of these deviations were clinically significant. Clinically significant laboratory result deviations were recorded as AEs and the relationship to the treatment was indicated.

### Vital Signs

Systolic and diastolic blood pressure and pulse were recorded after the subject had been resting for at least 5 minutes in the supine position. These assessments were made using an automated device. Body temperature and respiratory rate were measured subsequently.

### Electrocardiogram

A standard 12-lead ECG was recorded after the subject had been resting for at least 5 minutes in the supine position. The ECG was recorded using an ECG machine equipped with computer-based interval measurements (with no/minimal disturbance by procedures). The following ECG parameters were recorded: heart rate, PR-interval, QRS-duration, QT-interval, QTcF-interval, and the interpretation of the ECG profile by the Investigator.

### Continuous Cardiac Monitoring (Telemetry)

In the SAD part (not in the second period of the FE group A4, and not in Group A5), a 12-lead ECG was recorded continuously by telemetry from 2 hours before to 24 hours after drug administration on Day 1.

In the MAD part, a 12-lead ECG was recorded continuously by telemetry from 2 hours before to 12 hours after drug administration on Day 1, and from 2 hours before to 24 hours after drug administration on Day 14.

All relevant or significant arrhythmic events were recorded in rhythm strips (10 seconds). The ECG was evaluated by the Investigator for clinically significant events.

During days with telemetry, meals were standardized, and subjects remained quietly supine (with no/minimal disturbance by procedures) for 10 minutes followed by an up to 5-minute period for each ECG assessment that was planned just prior to PK sampling. Start and stop time of the (in total) 15-minute periods were recorded. The ECGs collected by continuous monitoring (telemetry) were stored for potential later use.

These ECGs may or may not be analyzed for the purpose of concentration-effect modeling, based on future development decisions for IMB-1018972. If analyzed, results of the modeling were not to be included in this CSR, but to be included in a separate report.

### Physical Examination

Physical examination was performed according to PRA SOPs. In addition, body weight and height were measured according to PRA SOPs.

### Pharmacokinetic Measurements

### Blood Sampling

At the time points defined in the schedules of assessments, blood samples of 3 mL per time point were taken for the analysis of IMB-1018972, IMB-1028814, and trimetazidine in plasma samples. The blood samples were taken via an indwelling intravenous catheter or by direct venipuncture. The exact times of blood sampling were recorded in the eCRF.

During days with telemetry, subjects remained quietly supine (with no/minimal disturbance by procedures) for 10 minutes followed by an up to 5-minute period for each ECG assessment that was planned just prior to PK sampling. Start and stop time of the (in total) 15-minute periods were recorded.

Details on sample collection, sample aliquoting, sample handling, sample storage, and sample shipping can be found in the laboratory manual prepared by PRA.

Plasma samples may (in the future) also be used for research purposes such as evaluation of the activity of IMB-1018972 and trimetazidine, identification of exploratory biomarkers that are predictive of activity, cytochrome P450 profiling, or other exploratory evaluations that may help characterize the molecular mechanisms of IMB-1018972 and trimetazidine. The samples will be stored for a maximum of 15 years for this purpose.

### Urine Collection

Urine collection for PK was only conducted in the SAD part, but not in the second period of the FE group A4.

During the intervals defined in the schedules of assessments, urine was collected for the analysis of IMB-1018972, IMB-1028814, and trimetazidine. The subjects were instructed to empty their bladders completely before study drug administration and at the end of each collection interval. A blank urine sample was collected within 12 hours prior to study drug administration. The exact times of urine collection and the urine weight of the entire interval (before and after addition of any urine stabilizers, if used) were recorded in the eCRF.

Details on sample collection, sample aliquoting, sample handling, sample storage, and sample shipping can be found in the laboratory manual prepared by PRA.

Urine samples could be kept for a maximum of 1 year for further analysis of metabolites in urine in case unknown metabolites were found in plasma.

### Genotyping

At the time points defined in the schedules of assessments, a blood sample of a maximum of 7 mL was collected for genotyping to better understand the effects of genotype, such as CYP alleles, on PK data. This blood sample was optional for subjects that had already been screened prior to IEC approval of protocol Version 3.0 (25 Mar. 2019), whereas it was mandatory for subjects participating in this study that had been screened after IEC approval of protocol Version 3.0 (25 Mar. 2019).

The blood sample was double coded (1 code at PRA and 1 code at the Sponsor), and the sample was kept until 15 years after completion of the study.

The blood sample was taken via an indwelling intravenous catheter or by direct venipuncture. The exact time of blood sampling was recorded in the eCRF.

Details on sample collection, sample aliquoting, sample handling, sample storage, and sample shipping can be found in the laboratory manual prepared by PRA.

### Safety and Pharmacokinetic Variables

The safety variables to be measured included:
AEs
Clinical laboratory
Vital signs
12-lead ECG
Continuous cardiac monitoring (telemetry)
Physical examination

### Pharmacokinetic Variables

Pharmacokinetic variables were the plasma and urine concentrations of IMB-1018972, IMB-1028814, and trimetazidine, and their PK parameters. The PK parameters that were determined or calculated using noncompartmental analysis are given in Table

TABLE 8

Plasma IMB-1018972, IMB-1028814, and Trimetazidine Parameters

| | | | | |
|---|---|---|---|---|
| Parameter | SAD/FE | MAD Day 1 | MAD Day 14 | Description |
| Cmax | X | X | X | Maximum plasma concentration. Observed peak analyte concentration obtained directly from the experimental data without interpolation, expressed in concentration units. |
| Cmin | | | X | Minimum plasma concentration (predose concentration excluded). |
| tmax | X | X | X | Time to maximum plasma concentration. First observed time to reach peak analyte concentration obtained directly from the experimental data without interpolation, expressed in time units. |
| AUC₀₋ₜ | X | | | Area under the plasma concentration-time curve (time 0 to time of last quantifiable concentration). |
| AUC_{0-inf} | X | | | Area under the plasma concentration-time curve (time 0 to infinity). Percent extrapolation less than or equal to 20% is required to obtain a reliable AUC_{0-inf} |
| %AUCextra | X | | | Percentage of estimated part of the calculation of AUC_{0-inf}. Calculated as: ([AUC_{0-inf}-AUC₀₋ₜ]/AUC_{0-inf})* 100%. |
| AUC_{0-T} | | X | X | Area under the plasma concentration-time curve over the dosing interval of 0-12 hours postmorning dose. |
| kₑₗ | X | | X | Terminal elimination rate constant calculated by linear regression of the terminal log-linear portion of the concentration vs time curve. Linear regression of at least 3 points and an adjusted r² greater than 0.80 were required to obtain a reliable kₑₗ. |
| tl/2 | X | | X | Terminal elimination half-life expressed in time units. Percent extrapolation less than or equal to 20% and adjusted r² greater than 0.80 was required to obtain a reliable t_{½}. |
| CL/F | X | | | Apparent oral clearance, calculated as dose/AUC_{0-inf} IMB-1028814 only, assuming 100% IMB-1018972 was converted to IMB-1028814. |
| CLₛₛ/F | | | X | Apparent oral clearance at steady state, calculated as dose/AUC_{0-T}. The AUC_{0-T} after the morning dose was used in the calculation. IMB-1028814 only, assuming 100% IMB-1018972 was converted to IMB-1028814. |
| V_{z}/F | X | | X | Apparent volume of distribution at terminal phase, calculated as (CL/F)/kₑₗ (SAD/FE/MR), or as (CLₛₛ/F)/kₑₗ (MAD). For IMB-1028814 only. |
| R_{ac} | | | X | Accumulation ratio, based on AUC_{0-T} of Day 14 vs Day 1 (AUC_{0-T} after morning dose). |

FE=food effect; MAD=multiple ascending dose; SAD=single ascending dose; MD=multiple dose; MR=modified release; SD=single dose

The sum of IMB-1028814 and trimetazidine concentrations and PK parameters was calculated corrected for molecular weights of 310 kDa for IMB-1028814 and 266 kDa for trimetazidine.

Plasma trough levels of IMB-1018972, IMB-1028814, and trimetazidine were also determined (MAD part only).

The AUCs were calculated using the linear up/log down trapezoidal rule, expressed in units of concentration x time.

TABLE 9

Urine IMB-1018972, IMB-1028814, and Trimetazidine Parameters

| | | |
|---|---|---|
| Parameter | SAD/FE (first period) | Description |
| Aeᵤᵣᵢₙₑ | X | Total amount of drug excreted unchanged into urine to time t (time of last measurable concentration), obtained by adding the amounts excreted over each collection interval. |
| Feᵤᵣᵢₙₑ | X | Fraction (%) of the administered dose excreted unchanged into urine. Calculated as: Feᵤᵣᵢₙₑ=(Aeᵤᵣᵢₙₑ/Dose) * 100. |
| CL_{R} | X | Renal clearance. Calculated as Aeᵤᵣᵢₙₑ/AUC₀₋ₜ. |

### Drug Concentration Measurements

The analysis of IMB1018972, IMB-1028814, and trimetazidine in plasma and urine samples was performed at the Bioanalytical Laboratory of PRA using validated liquid chromatography-mass spectrometry/mass spectrometry methods. The results from calibration samples and quality control samples demonstrated acceptable performance of the methods throughout the experimental period. Data on the performance of the method and stability indicate that the sample results as reported are reliable.

### Statistical and Analytical Plan for Safety and Pharmacokinetic Evolution

### Safety Set

All subjects who had received at least 1 dose of IMB1018972, trimetazidine, or placebo.

### Pharmacokinetic Set

All subjects who had received at least 1 dose of IMB-1018972 or trimetazidine and provided sufficient bioanalytical assessment results to calculate reliable estimates of the PK parameters.

### Statistical and Analytical Plan for Safety and Pharmacokinetic Evaluation

Details on the preparation of the listings and summary tables and figures can be found in the SAP and was generated by the Biostatistics Department of PRA. The SAP was finalized prior to database lock (and unblinding of study treatment codes).

All safety and PK data were listed. In addition, all data were summarized in tabular and/or graphical form and descriptive statistics were given, as appropriate.

### Evolution of Safety and Tolerability

Safety and tolerability were assessed through AEs, clinical laboratory, vital signs, ECGs, continuous cardiac monitoring (telemetry), and physical examination findings, and any other parameter that was relevant for safety assessment.

All individual safety results were listed and descriptive statistics including change from baseline were calculated, where applicable.

### Pharmacokinetic Evaluation

Descriptive statistics (number, arithmetic mean, SD, coefficient of variation, minimum, maximum, median, and geometric mean) were calculated for plasma and urine PK parameters of IMB-1028814, trimetazidine, and IMB-1028814 + trimetazidine in the PK population, where applicable.

Dose proportionality of IMB-1018972, IMB-1028814 and trimetazidine was explored for SAD Groups A1 to A4 (fasted) using a regression (power) model relating log-transformed Cmax, AUC₀₋ₜ, and AUC_{0-inf}. Subjects with R2 below 0.80 or %AUCextra>20% were not excluded from the dose-proportionality evaluation based on AUC_{0-inf}. A point estimate and 95% CI were produced for the slope. A slope of 1 (i.e., a 95% CI containing 1) means that no evidence of a deviation from dose proportionality was found. Since there were only 2 dose levels in the MAD part, no dose-proportionality analysis was performed for the MAD part.

The effect of food on the relative oral bioavailability of IMB-1018972 following a single oral administration was explored. This occurred in Group A4 of the SAD part where subjects received the same dose, first under fasted conditions and then under fed conditions. The evaluation was based on 90% CIs for the ratio of the geometric means, based on log-transformed data, for Cₘₐₓ, AUC₀₋ₜ, and AUC_{0-inf}.

### Determination of Sample Size

For this FIH study, no prospective calculations of statistical power were made. The sample size was selected to provide information on safety, tolerability, and PK following single and multiple doses of IMB-1018972, single doses of trimetazidine, and is typical for a FIH study. Any p-values to be calculated according to the SAP were interpreted in the perspective of the explorative character of this study.

### Study Subjects

Of the 220 subjects who were screened, 88 subjects were included in the study and received the study drug. Sixty-six subjects received a dose of IMB-1018972, 8 received trimetazidine, and 14 received placebo.

Eighty-five of 88 subjects completed the study. Subject 129 of the FE arm Group A4 withdrew consent on Day 1 of the second period after receiving the single oral dose of 150 mg IMB-1018972 under fed conditions. Subject 131 of the FE arm Group A4 was withdrawn from the study due to an SAE of influenza like illness (of moderate severity and unlikely related) in the first period and only received the single oral dose of 150 mg IMB-1018972 under fasted conditions and not the fed dose in the second treatment period.

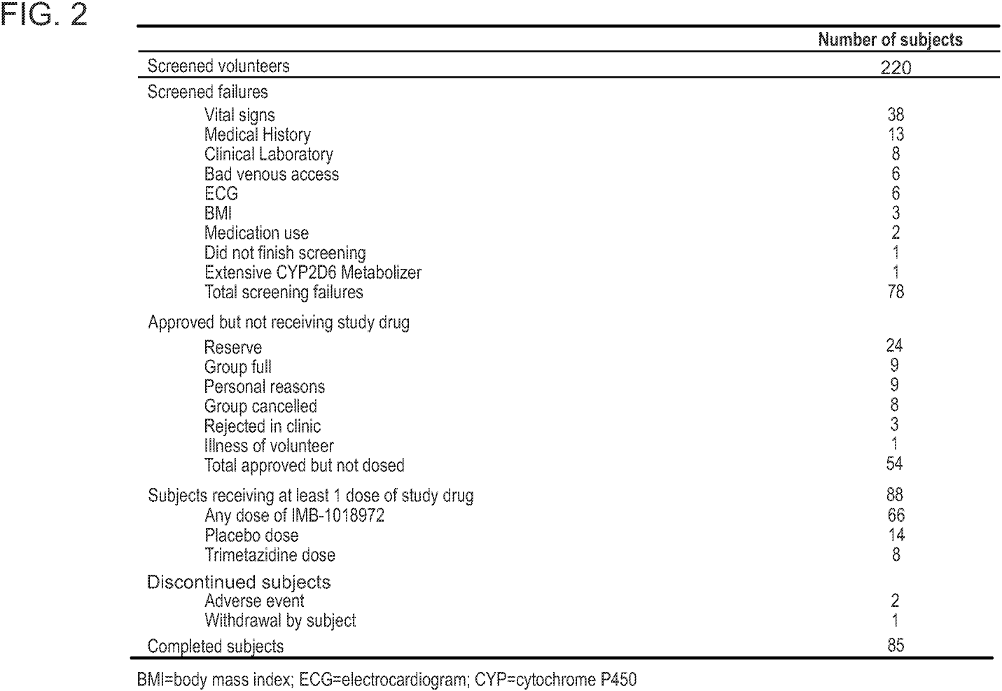

FIG. 2 is a table of the disposition of subjects.

### Protocol Deviations/Violations

Several protocol deviations that were not deemed significant are described in the listings given above; these are not further described in this section.

Two major deviations were recorded in the study:
There was 1 major deviation from the GCP and PRA procedures. Three volunteers were screened 1 day before the study initiation visit was conducted. This deviation was not considered to have had any implications for the safety of the involved volunteers.
The pharmacy at PRA had only received an original set of randomization lists for the SAD part with the treatments as described in Versions 2.0 and 3.0 of the CSP. They did not receive updated randomization lists based on Versions 4.0 and 5.0 of the CSP which changed the designs of Groups A4 and A5. Similarly, the Bioanalytical Laboratory of PRA also did not receive the updated randomization lists from the pharmacy. However, all subjects of Groups A4 and A5 received the correct dose and therefore, this deviation did not have any implications for the safety of the involved volunteers.

In addition, a memo to file, dated 22 Oct. 2019, was issued documenting the following protocol deviations:
For all subjects of Groups B1 and B2 of the MAD part, vital signs on Day 1 at 12 hours after the morning dose were recorded prior to instead of after the ECG and PK blood sampling because of the risk that the evening dose could not be administered in time.
For all subjects of Groups B1 and B2 of the MAD part, vital signs on Day 14 in the morning were scheduled 25 minutes earlier than planned because of the risk that the morning dose could not be administered in time.

### Genotyping

Except for 1 subject in the SAD part, all subjects provided a blood sample for genotyping. The blood sample was used to genotype subjects with a particular interest on CYP2D6 to better understand differences in the PK data. Any results of the analysis of the relationship between genotype and PK data will presented separately from this CSR.

### Measurements of Treatment Compliance

Study drug was administered in the clinical research center. To ensure treatment compliance, administration of the study drug was supervised by the Investigator or authorized designee. There was no indication of noncompliance based on observations during study drug administration. In addition, bioanalytical assessment of IMB-1018972, IMB-1028814, and trimetazidine in plasma and urine samples confirmed treatment compliance.

### Clinical Laboratory Evaluation

### Laboratory Values Over Time

Although several individual changes from baseline were observed in the clinical laboratory values, no clinically important trends were seen.

### Individual Subject Changes

The majority of the subjects had one or more out of range values for clinical laboratory tests at various times during the study. Most of these were minor and considered by the Investigator to have no clinical implication. A number of ALT levels measured for 1 subject were above the normal range and considered to be clinically significant abnormal.

### Vital Signs, ECGs, Physical Findings, and Other Observations Related to Safety

### Vital Signs

Although several individual changes from baseline were observed, blood pressure, pulse, body temperature, and respiratory rate showed no trends or clinically relevant changes during any of the study parts.

### Electrocardiogram

No changes or trends of clinical significance were seen for the heart rate, PR-interval, QRS-duration, QT-interval, or QTcF-interval during any of the study parts. All 12-lead ECG evaluations were recorded as normal or, in case of abnormal recordings, these were not considered to be clinically significant.

### Continuous Cardiac Monitoring (Telemetry)

All telemetric ECG evaluations obtained in the SAD and MAD parts were recorded as normal or, in case of abnormal recordings, these were not considered to be clinically significant.

### Physical Examination

All abnormalities observed at screening and all changes observed after screening for physical examinations were considered to be of no clinical significance.

### Detailed Results for Immediate Release Formulations

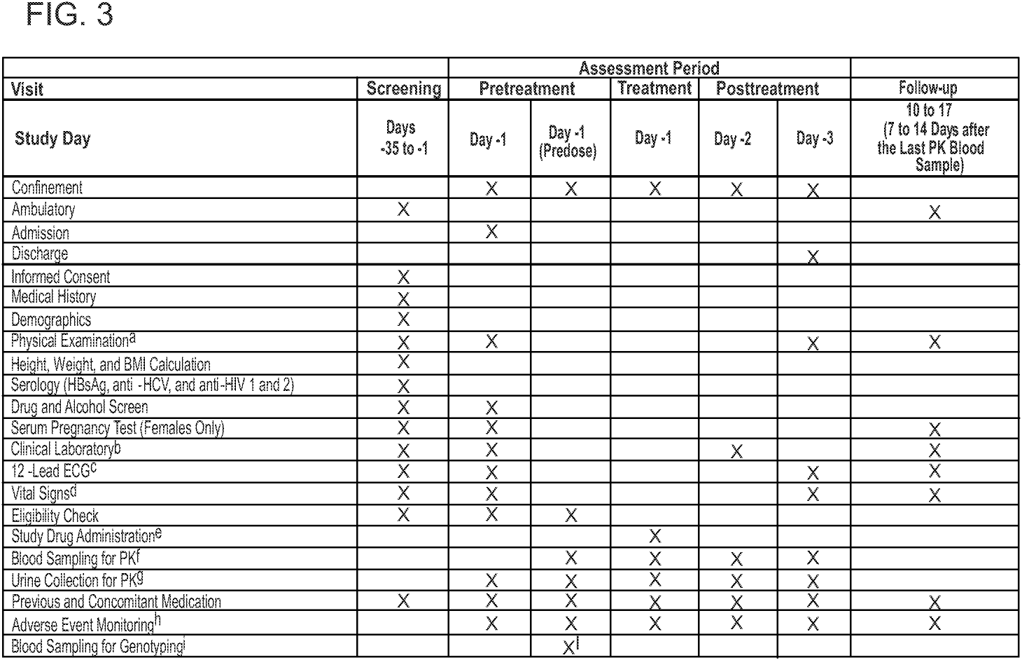

FIG. 3 is a Schedule of Assessments for SAD part Group A5, with the following notations:
BMI=body mass index;
ECG=electrocardiogram;
HBsAg=hepatitis B surface antigen;
HCV=hepatitis C virus;
PK=pharmacokinetic(s)

a. Physical examination: at screening, on Day -1 (admission; this was a directed examination only done at the discretion of the Investigator), at discharge on Day 3 (this was a directed examination only done at the discretion of the Investigator), and at follow-up.
b. Clinical laboratory tests (including clinical chemistry, hematology, coagulation, and urinalysis): at screening, on Day -1 (admission) and at 24 hours postdose, and at follow-up.
c. 12-lead ECG at screening, on Day -1 (admission), at 48 hours postdose, and at follow-up.
d. Vital signs (supine systolic and diastolic blood pressure, pulse, body temperature, and respiratory rate): at screening, on Day -1 (admission), at 48 hours postdose, and at follow-up. e Study drug administration was conducted under fasted conditions

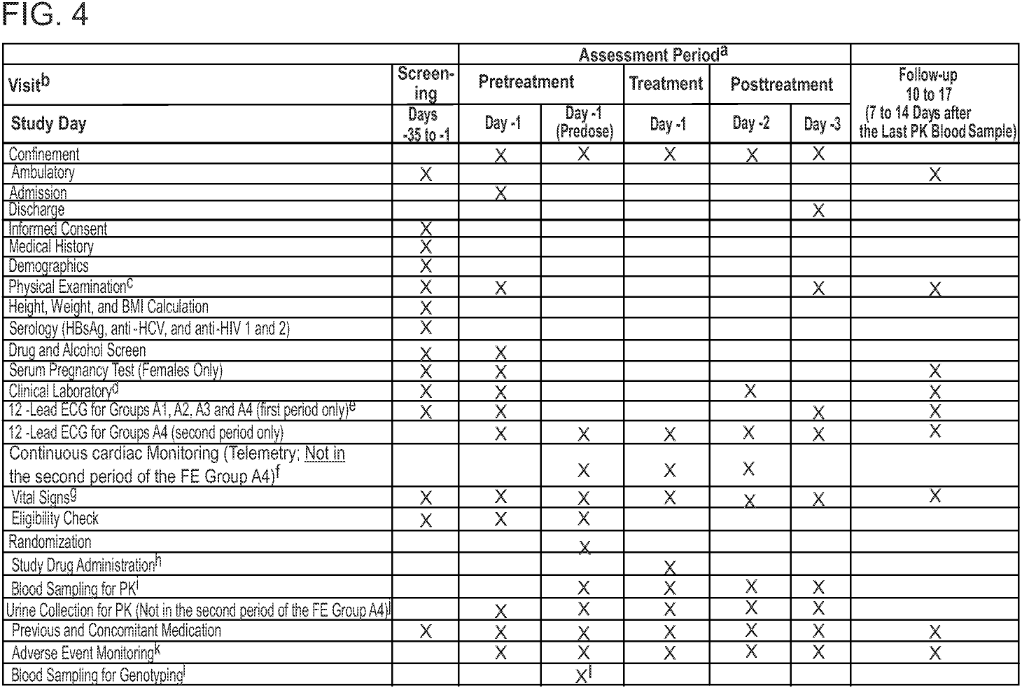

FIG. 4 is a table of assessments given for the SAD part (and integrated FE arm) Groups A1 to A4, with the following notations:
BMI=body mass index
ECG=electrocardiogram;
FDA=Food and Drug Administration;
FE=food effect;
HBsAg=hepatitis B surface antigen;
HCV=hepatitis C virus;
PK=pharmacokinetic(s); SAD=single ascending dose

a. Subjects were in the clinic for 1 period, except for subjects of Group A4 also participating in the FE arm who were in the clinic for 2 periods; a period was from Day -1 until 48 hours (Day 3) postdose.
b. The planned confinement period, day of discharge, and follow-up period could be adapted depending on emerging study results. Also, the timing, type, and number of safety and PK assessments could be changed during the study.
c. Physical examination: at screening, each period on Day -1 (admission; this was a directed examination only done at the discretion of the Investigator), at discharge on Day 3 (this was a directed examination only done at the discretion of the Investigator), and at follow-up.
d. Clinical laboratory tests (including clinical chemistry, hematology, coagulation, and urinalysis): at screening, each period on Day -1 (admission) and at 24 hours postdose, and at follow-up.
e. 12-lead ECG for Groups A1, A2, A3, and A4 (first period only): at screening, on Day -1 (admission), at 48 hours postdose, and at follow-up. Data for 12-lead ECGs at predose and 1, 2, 4, 6, 12, and 24 hours postdose were taken from the 12-lead ECG prints from telemetry. The predose baseline value was the average values of the 3 predose telemetry 12-lead ECGs at -1.25, -1.0, and -0.75 hours predose. 12-lead ECG for Group A4 (second period only): on Day -1 (admission), at predose and 1, 2, 4, 6, 12, 24, and 48 hours postdose, and at follow-up.
f. Only in the SAD part; not in the second period of Group A4 also participating in the FE arm: Continuous cardiac monitoring (12-lead telemetry): from at least 2 hours predose until at least 24 hours postdose. 12-lead ECG reads were printed at -1.25, -1.0, and -0.75 hours predose and just prior to the PK sampling time points of 0.25, 0.5, 1, 2, 4, 6, 12, and 24 (Day 2) hours postdose.
g. Vital signs (supine systolic and diastolic blood pressure, pulse, body temperature, and respiratory rate): at screening, each period on Day -1 (admission), each period at predose and 1, 2, 4, 6, 12, 24, and 48 hours postdose, and at follow-up.
h. In Groups A1, A2, A3, and, A4, and in the first period of Group A4 also participating in the FE arm, study drug administration was conducted under fasted conditions. In the second period of Group A4, drug administration was conducted under fed conditions (FDA-defined high-fat breakfast).
i. Blood sampling for PK of IMB-1018972, IMB-1028814, and trimetazidine in plasma: each period at predose and 0.25, 0.5, 1, 2, 3, 4, 5, 6, 8, 10, 12, 16, 24, 36, and 48 hours postdose.
j. Only in the SAD part; not in the second period of Group A4 also participating in the FE arm: Urine collection for PK of IMB-1018972, IMB-1028814, and trimetazidine in urine: each period at predose (within 12 hours prior to dosing) and over 0-6, 6-12, 12-24, 24-36, and 36-48 hours postdose collection intervals.
k. AEs were recorded from (first) admission until completion of the follow-up visit. 1 Blood sampling for genotyping was optional for subjects that had already been screened prior to IEC approval of protocol Version 3.0 (25 Mar. 2019), whereas it was mandatory for subjects participating in this study that had been screened after IEC approval of protocol Version 3.0 (25 Mar. 2019). For the subjects for which this sample was mandatory, the sample was taken on Day 1 of the first period only. For the subjects for which this sample was optional and who consented to provide this sample, the sample could be taken on any day during the study; a separate visit could be planned after follow-up to take this sample, if needed.

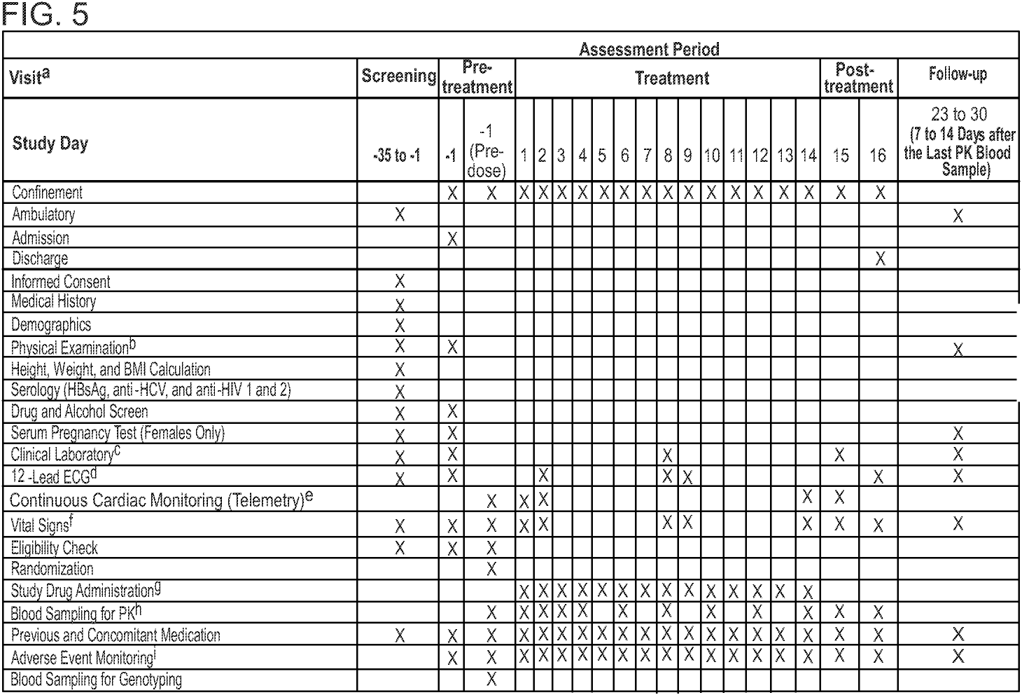

FIG. 5 is a table of assessments given for the MAD part, with the following notations:
BMI=body mass index;
ECG=electrocardiogram;
FE=food effect;
HBsAg=hepatitis B surface antigen;
HCV=hepatitis C virus;
MAD=multiple ascending dose;
PK=pharmacokinetic(s);
q8h=every 8 hours; q12h=every 12 hours; qd=once daily;
SAD=single ascending dose; tid=three times a day

a. The planned confinement period, day of discharge, and follow-up period could be adapted depending on emerging study results. Also, the timing, type, and number of safety and PK assessments could be changed during the study.
b. Physical examination: at screening, on Day -1 (admission; this was a directed examination only done at the discretion of the Investigator), and at follow-up. On other days, a physical examination could be done on indication only at the discretion of the Investigator.
c. Clinical laboratory tests (including clinical chemistry, hematology, coagulation, and urinalysis): at screening, on Day -1 (admission), before the morning dose on Day 8 and at the same time on Day 15, and at follow-up.
d. 12-lead ECG: at screening, on Day -1 (admission), on Day 1 at 24 hours after the morning dose, on Day 8 at predose and 1, 2, 4, 6, 12 (prior to the evening dose) and 24 hours after the morning dose, on Day 16 (day of discharge) at the same time as before the morning dose on dosing days, and at follow-up. Data for 12-lead ECGs on Day 1 at predose and 1, 2, 4, 6, and 12 hours postdose, and on Day 14 at predose and 1, 2, 4, 6, 12, and 24 hours postdose were taken from the 12-lead ECG prints from telemetry. The predose baseline value on Day 1 and Day 14 was the respective average values of the 3 predose telemetry 12-lead ECGs at -1.25, -1.0, and -0.75 hours predose.
e. Continuous cardiac monitoring (12-lead telemetry): from at least 2 hours before the morning dose until at least 12 hours after the morning dose on Day 1 and until at least 24 hours after the morning dose on Day 14. 12-lead ECG reads were printed at -1.25, -1.0, and -0.75 hours before the morning dose and just prior to the PK sampling time points of 0.25, 0.5, 1, 2, 4, 6, 12, and 24 (Day 14 only) hours after the morning dose.
f. Vital signs (supine systolic and diastolic blood pressure, pulse, body temperature, and respiratory rate): at screening, on Day -1 (admission), on Days 1, 8, and 14 at predose and 1, 2, 4, 6, 12 (prior to the evening dose on Days 1 and 8) and 24 hours after the morning dose, on Day 16 (day of discharge) at the same time as before the morning dose on dosing days, and at follow-up.
g. The study drug was administered twice daily for 14 days; on Day 14 only a single morning dose wasadministered. Study drug administration was conducted under fed conditions as determined based on the results of Group A4 in the FE arm. Note: The study drug was given for 14 consecutive days, but this could be revised based on the safety and tolerability results (and plasma PK results, if available) of the SAD part and of previous group(s) in the MAD part. Similarly, it could be decided to change q12h dosing to qd or tid (q8h) dosing.
h. Blood sampling for PK of IMB-1018972, IMB-1028814, and trimetazidine in plasma: on Days 1 and 14 before the morning dose and at 0.25, 0.5, 1, 2, 3, 4, 5, 6, 8, 10, 12 (prior to the evening dose on Day 1), 16, 24, 36, and 48 hours after the morning dose, and on Days 4, 6, 8, 10, and 12 before the morning dose.
i. AEs were recorded from admission until completion of the follow-up visit.
j. Blood sampling for genotyping was mandatory.

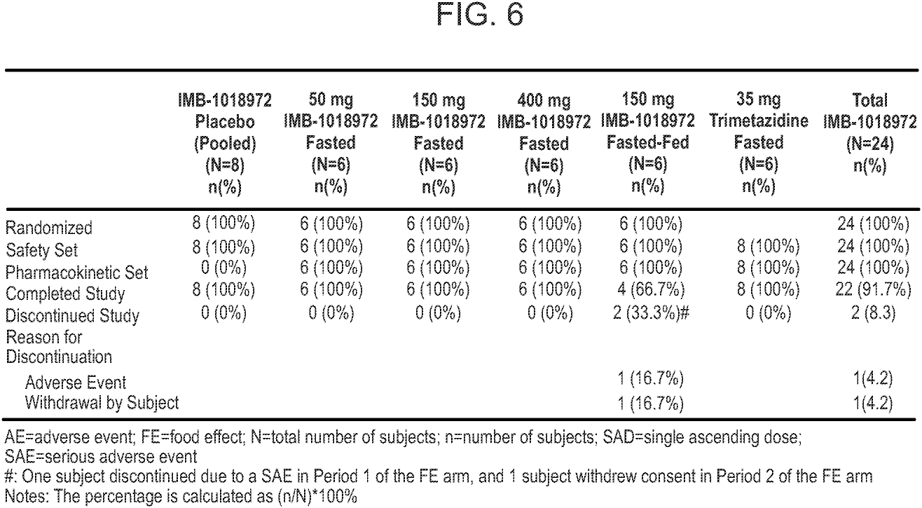

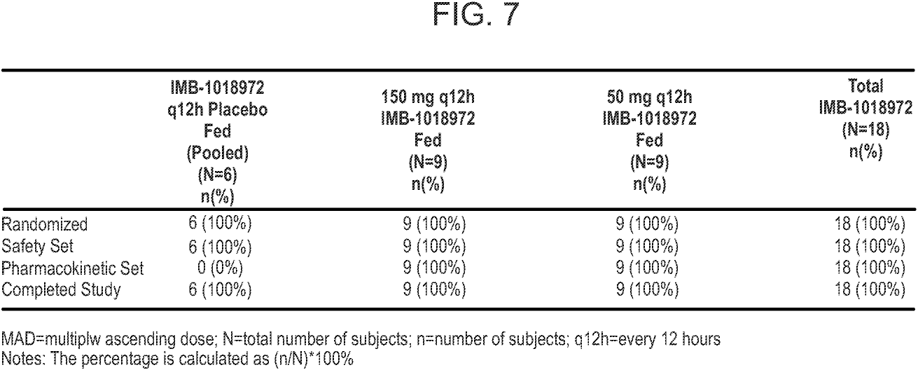

FIG. 6 is a table of analysis data sets for the SAD Part (and integrated FE Arm) per dose level and total for IMB-1018972.

FIG. 7 is a table of analysis data sets for the MAD Part per dose level and total for IMB-1018972.

### Demographic and Other Baseline Characteristics

### SAD Part (and Integrated FE Arm)

In the SAD part with integrated FE arm, a total of 40 subjects were included.

### IMB-1018972 and Placebo

Thirty-two subjects were included of whom 23 were female and 9 were male Mean age ranged between 29 and 46 years and mean BMI ranged between 23.0 and 26.6 kg/m2 over all treatments, including placebo. Individual age ranged between 18 and 65 years and individual BMI ranged between 19.5 and 30.3 kg/m2. Twenty-nine subjects were of white race, 1 subject was Asian, 1 subject was Black or African American, and 1 subject was Native Hawaiian or Other Pacific Islander. Thirty-one subjects were not of Hispanic or Latino ethnicity whereas 1 subject was of Hispanic or Latino ethnicity. The summary of the PK set was identical to that of the safety set minus the pooled placebo group.

### Trimetazidine Group

Eight subjects were included of whom 5 were female and 3 were male. Mean age was 32 years and mean BMI was 23.7 kg/m2. Individual age ranged between 20 and 65 years and individual BMI ranged between 19.4 and 26.7 kg/m2. Seven subjects were of white race and 1 subject was of multiple race. Seven subjects were not of Hispanic or Latino ethnicity whereas 1 subject was of Hispanic or Latino ethnicity. The summary of the PK set was identical to that of the safety set.

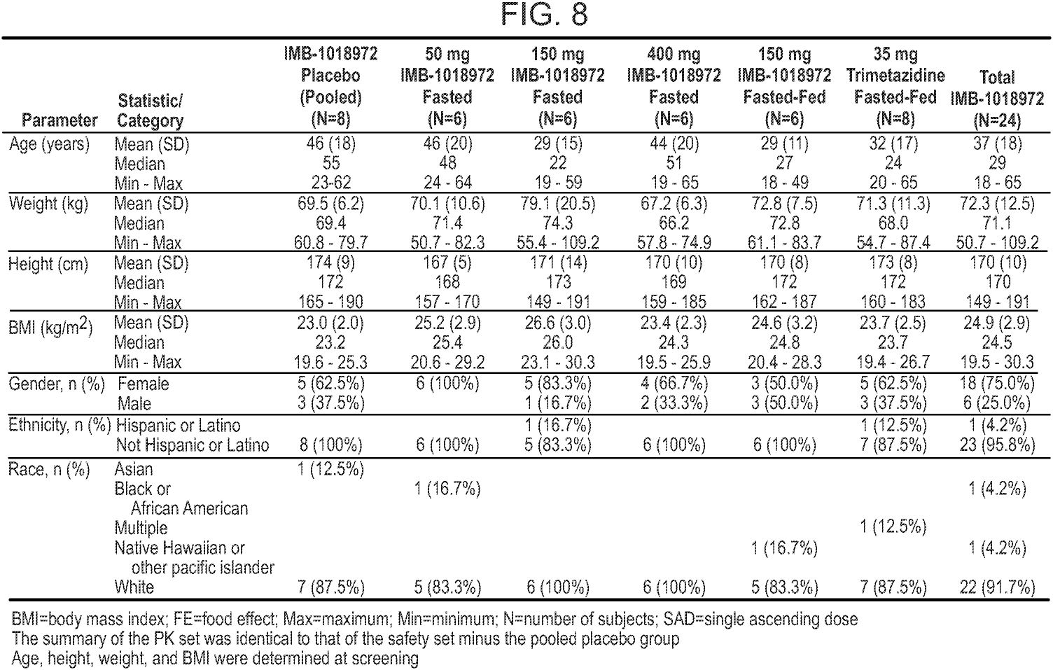

FIG. 8 is a table of a summary of demographic characteristics - SAD Part (and Integrated FE Arm) (Safety Set).

### MAD Part

Twenty-four subjects were included of whom 12 were female and 12 were male. Mean age ranged between 38 and 44 years and mean BMI ranged between 25.2 and 26.7 kg/m2 over all treatments, including placebo. Individual age ranged between 18 and 64 years and individual BMI ranged between 19.1 and 30.9 kg/m2. Eighteen subjects were of white race, 2 subjects were of multiple race, 2 subjects were American Indian or Alaska Native, 1 subject was Asian, and 1 subject was Black or African American. Twenty-one subjects were not of Hispanic or Latino ethnicity whereas 3 subjects were of Hispanic or Latino ethnicity. The summary of the PK set was identical to that of the safety set minus the pooled placebo group.

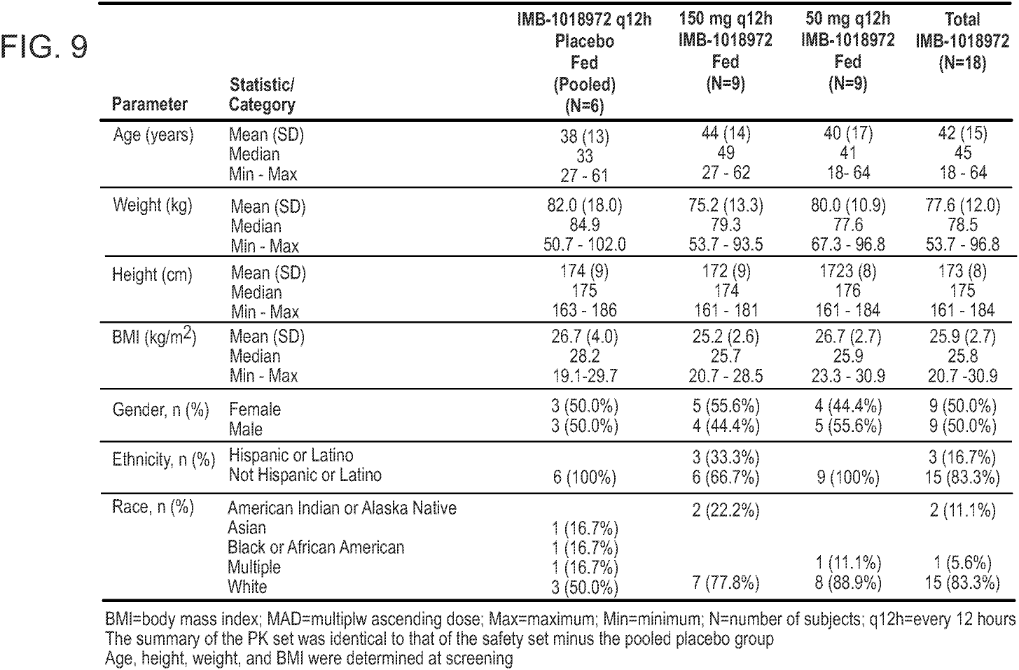

FIG. 9 is a table of a summary of demographic characteristics - MAD Part (Safety Set).

### Other Baseline Characteristics

All subjects complied with the inclusion and exclusion criteria. There were no clinically significant findings with regard to medical history or previous medication. Drug and alcohol screen results were negative for all subjects at screening and (each) admission. The results for the serology parameters were negative at screening for all subjects. The pregnancy test results were negative at screening, (each) admission, and follow-up for all females participating in this study.

### Extent of Exposure

A total of 88 subjects were dosed in this study: 40 subjects in the SAD part with integrated FE arm, 24 subjects in the MAD part.

In each of Groups A1, A2, A3 and A4 of the SAD part, 6 subjects received a single dose of IMB-1018972 and 2 subjects received a single dose of matching placebo under fasted conditions. IMB-1018972 doses ranged from 50 mg to 400 mg over these 4 groups. Subjects of SAD Groups A1, A2, and A3 participated in 1 single-dose treatment period, and subjects of SAD Group A4 (the FE group) participated in 2 singledose treatment periods with fasted dosing in the first period and fed dosing in the second period. Subject 131 of FE Group A4 only received the fasted IMB-1018972 dose in the first treatment period and not the fed dose in the second treatment period since the subject was withdrawn from the study in the first period due to a moderate SAE of influenza like illness (unlikely related).

In Group A5 of the SAD part, 8 subjects received a single oral dose of 35 mg trimetazidine under fasted conditions.

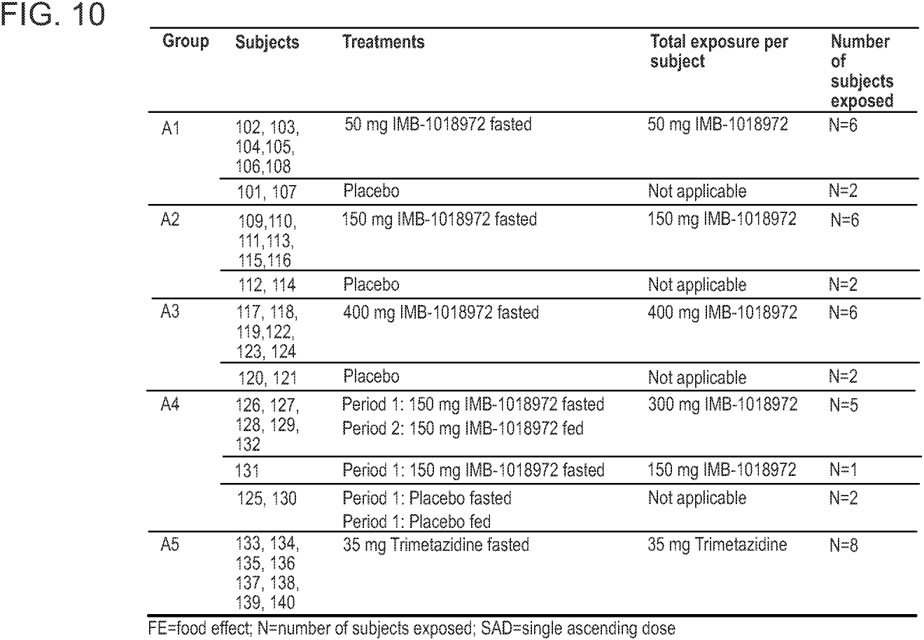

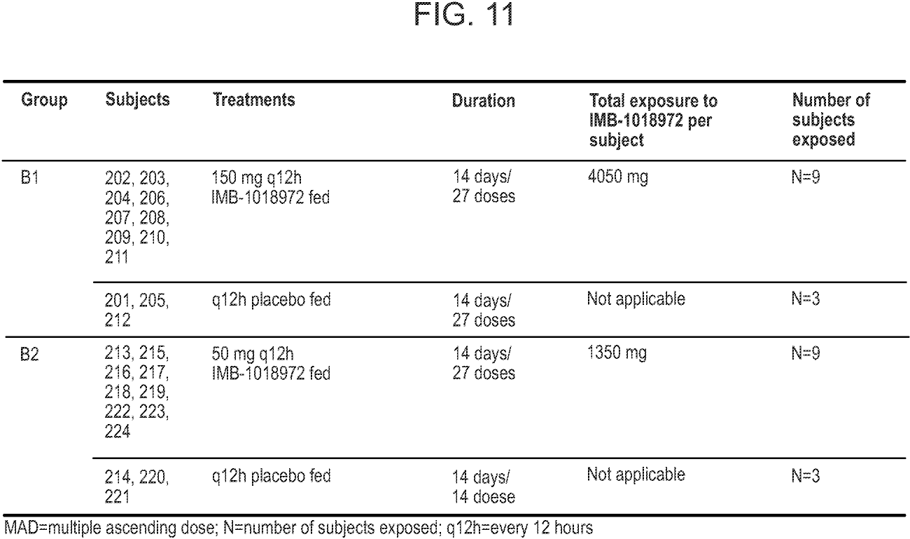

FIG. 10 is a table of the Extent of Exposure - SAD Part (and Integrated FE Arm) (Safety Set)

In both groups of the MAD part, 9 subjects received IMB-1018972 (150 mg for Group B1 and 50 mg for Group B2) and 3 subjects received matching placebo under fed conditions. In both groups, multiple oral doses of IMB-1018972 or matching placebo were administered q12h on Days 1 to 13 followed by a single morning dose on Day 14.

FIG. 11 is a table of the Extent of Exposure - MAD Part

### Pharmacokinetic Evaluation

The lower limit of quantification (LLOQ) was 0.5 ng/mL for IMB-1018972, IMB-1028814 and trimetazidine plasma concentrations, 10 ng/mL for IMB-1028814 urine concentrations, and 50 ng/mL for trimetazidine urine concentrations.

When more than 50% of the plasma values at a particular time point were below LLOQ, geometric means were not determined.

All blood samples of subjects that received IMB-1018972 in this study were analyzed for IMB-1018972 in plasma, but IMB-1018972 could be measured in only few plasma samples. Therefore, the IMB-1018972 concentrations have only been listed and no descriptive statistics or concentration-time profiles have been presented in this CSR. In addition, no PK parameters have been calculated for plasma IMB-1018972. As a result, urine samples were not analyzed for IMB-1018972 concentrations. Since the pharmacodynamic effect of IMB-1028814 and trimetazidine is the same, data are presented for IMB-1028814 and trimetazidine individually, as well as for the sum of IMB-1028814 and trimetazidine concentrations. The sum of IMB-1028814 and trimetazidine concentrations and PK parameters was calculated corrected for molecular weights of 310 kDa for IMB-1028814 and 266 kDa for trimetazidine.

### SAD Part (and Integrated FE Arm)

### PK in Plasma Following Administration of IMB-1018972 Under Fasted Conditions

All predose samples were below the LLOQ for IMB-1028814 and trimetazidine plasma concentrations.

The geometric mean concentration-time profiles for IMB-1028814, metabolite trimetazidine, and IMB-1028814 + trimetazidine showed a clear dose-dependent increase in plasma concentrations following administration of single doses of IMB-1018972 under fasted conditions in the dose range of 50 mg to 400 mg IMB-1018972.

The initial hydrolysis of IMB-1018972 to IMB-1028814 and subsequent systemic bioavailability of IMB-1028814 was relatively rapid with detectable concentrations generally seen between 15 and 30 minutes postdose. Detectable concentrations for trimetazidine also generally appeared between 15 and 30 minutes postdose. Median tₘₐₓ was around 1 hour postdose for IMB-1028814, and between 1.5 hours and 2 hours postdose for trimetazidine over the studied single-dose range of 50 mg to 400 mg IMB-1018972 under fasted conditions. Median tₘₐₓ did not increase with increasing IMB-1018972 dose.

The geometric mean Cₘₐₓ increased with dose and ranged between 104 ng/mL and 870 ng/mL for IMB-1028814, between 36.9 ng/mL and 274 ng/mL for trimetazidine, and between 516 nmol/L and 3,839 nmol/L (molar units to account for differences in molecular weight) for IMB-1028814 + trimetazidine over the studied single-dose range of 50 mg to 400 mg IMB-1018972 under fasted conditions. Similarly, the geometric mean AUC0-t increased with dose and ranged between 290 ng.h/mL and 2,795 ng.h/mL for IMB-1028814, between 424 ng.h/mL and 3,305 ng.h/mL for trimetazidine, and between 2,970 nmol.h/L and 22,365 nmol.h/L for IMB-1028814 + trimetazidine over the studied single-dose range of 50 mg to 400 mg IMB-1018972 under fasted conditions. The predefined stopping criterion for IMB-1028814 plasma exposure of 417,733 and 652,849 ng.h/mL for males and females, respectively, was not reached by any of the subjects during the SAD part.

Elimination of IMB-1028814 took place in a biphasic fashion, whereas elimination of trimetazidine occurred in a monophasic fashion. The geometric mean t_{1/2} of IMB-1028814 was relatively short, ranging between 2.6 hours and 3 hours over the IMB-1018972 single-dose range under fasted conditions. For metabolite trimetazidine, geometric mean t1/2 was longer, ranging between 6.76 hours and 8 hours over the IMB-1018972 single-dose range under fasted conditions. Geometric mean t_{1/2} of IMB-1028814 and trimetazidine did not increase with increasing IMB-1018972 dose indicating that the PK of the 2 moieties was linear.

Detectable individual IMB-1028814 concentrations were observed until 10, 12, 16, or 24 hours postdose after 50 mg, and until 16 or 24 hours postdose after 150 mg and 400 mg IMB-101897. Detectable individual trimetazidine concentrations were observed until 24, 36, or 48 hours postdose after 50 mg, until 36 or 48 hours postdose after 150 mg, and until 48 hours postdose after 400 mg IMB-1018972.

An aberrant IMB-1028814 and trimetazidine concentration-time profile was observed for Subject 108 who had received a single oral dose of 50 mg IMB-1018972 under fasted conditions. IMB-1028814 and trimetazidine tₘₐₓ was much later for this subject (5.00 hours for IMB-1028814 and 8.00 hours for trimetazidine) than for the other subjects who received the same dose (between 0.50 and 1.02 hours for IMB-1028814 and between 1.00 and 2.00 hours for trimetazidine). Therefore, absorption of IMB-1018972 by this subject is much slower than for the other subjects who received the same dose.

Dose proportionality for IMB-1028814 and trimetazidine was explored by plotting the dose-normalized exposure parameters Cₘₐₓ, AUC₀₋ₜ, and AUC_{0-inf} on a linear scale. The 95% CIs of the slopes of all 3 exposure parameters included 1 for both IMB-1028814 and trimetazidine. This indicates that no evidence of a deviation from dose proportionality of IMB-1028814 and trimetazidine was found over the IMB-1018972 single-dose range of 50 to 400 mg.

### PK in Plasma Following Administration of Trimetazidine

All predose samples were below the LLOQ for trimetazidine plasma concentrations. Following administration of a single oral dose of 35 mg trimetazidine, detectable trimetazidine concentrations were generally seen between 15 and 30 minutes postdose. Median trimetazidine tₘₐₓ was 5 hours, and geometric mean values were 68.6 ng/mL for Cₘₐₓ, 912 ng.h/mL for AUC0-t, and 929 ng.h/mL for AUC_{0-inf}.

Elimination of trimetazidine occurred in a monophasic fashion up to the last time point above LLOQ with a geometric mean t_{½} of 7.49 hours. Detectable individual trimetazidine concentrations were observed until the last sampling time point of 48 hours postdose.

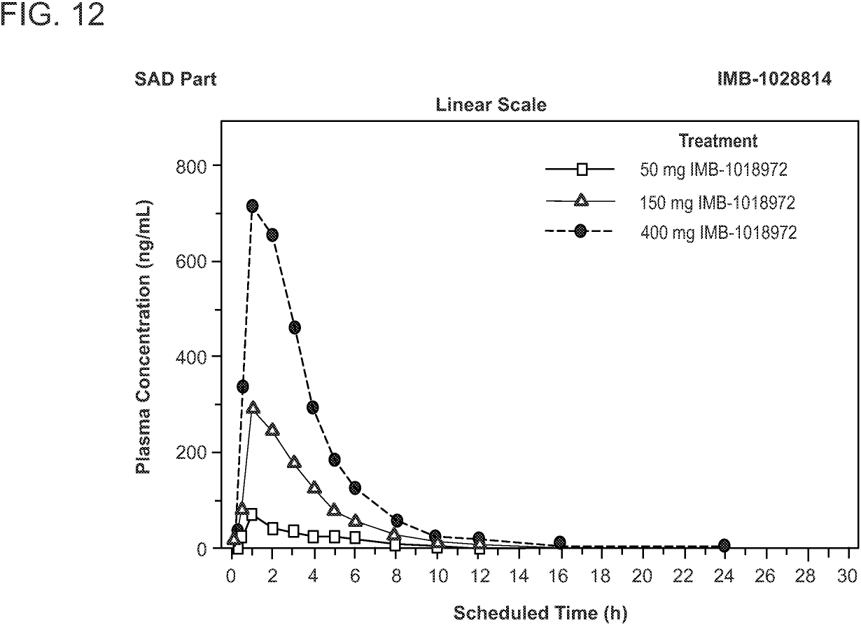

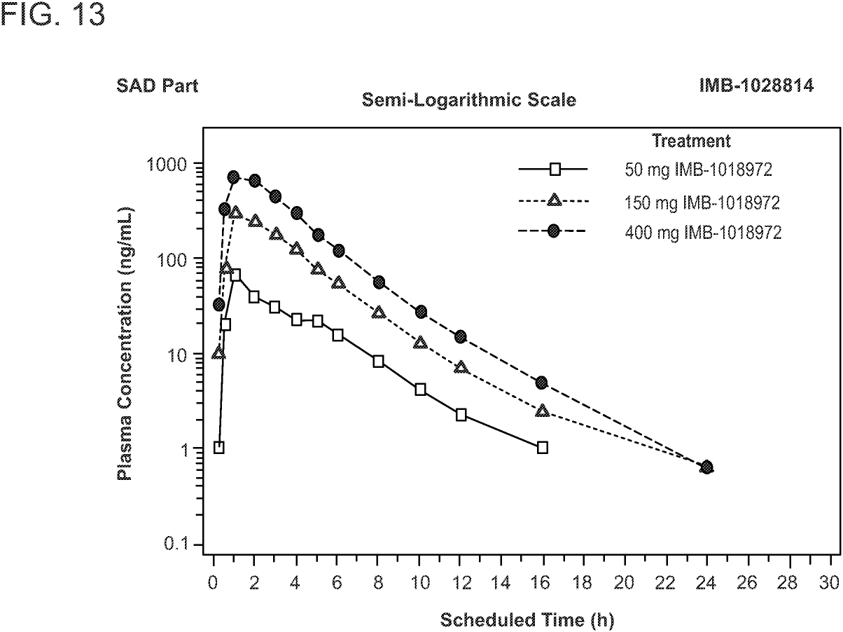

FIG. 12 is a graph of Geometric Mean IMB-1028814 Plasma Concentration-Time Profiles (Linear) - SAD Part (PK Set)

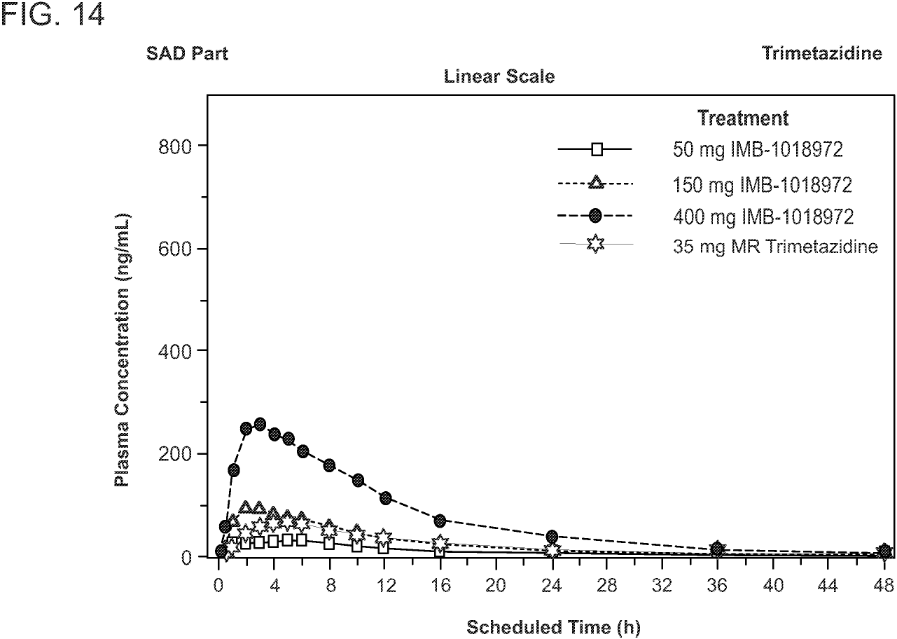

FIG. 13 is a graph of Geometric Mean IMB-1028814 Plasma Concentration-Time Profiles (Semi-Logarithmic) - SAD Part (PK Set)

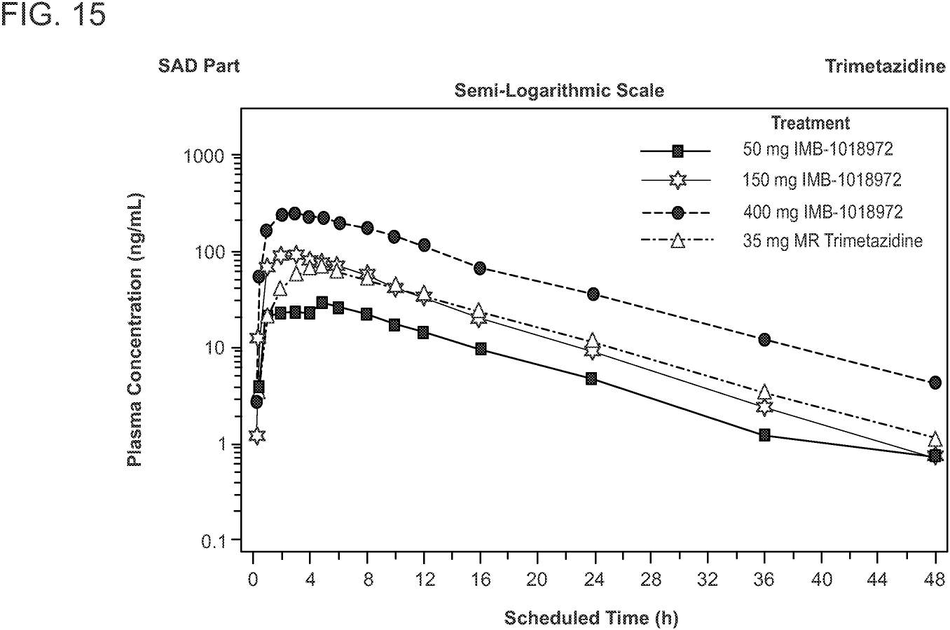

FIG. 14 is a graph of Geometric Mean Trimetazidine Plasma Concentration-Time Profiles (Linear) - SAD Part (PK Set)

FIG. 15 is a graph of Geometric Mean Trimetazidine Plasma Concentration-Time Profiles (Semi-Logarithmic) - SAD Part (PK Set)

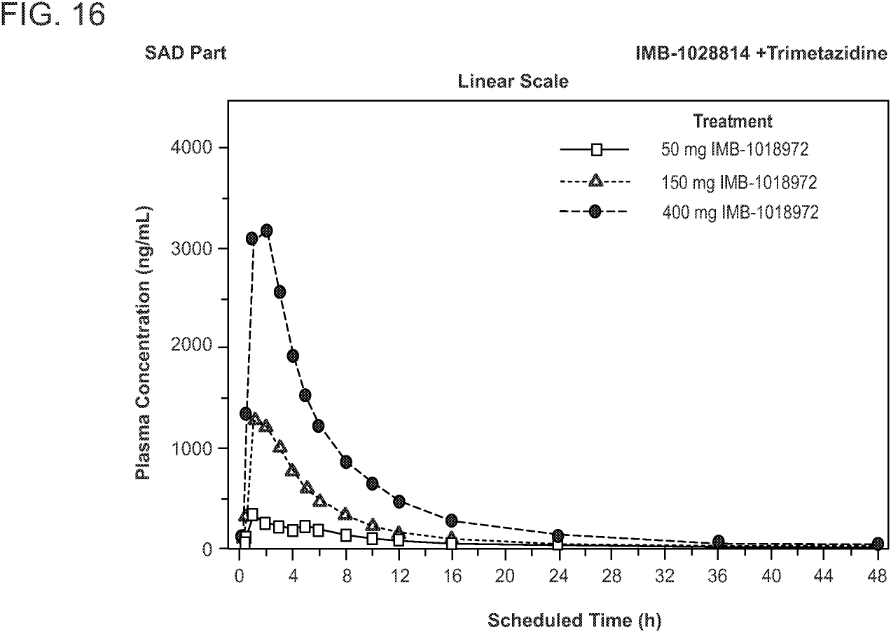

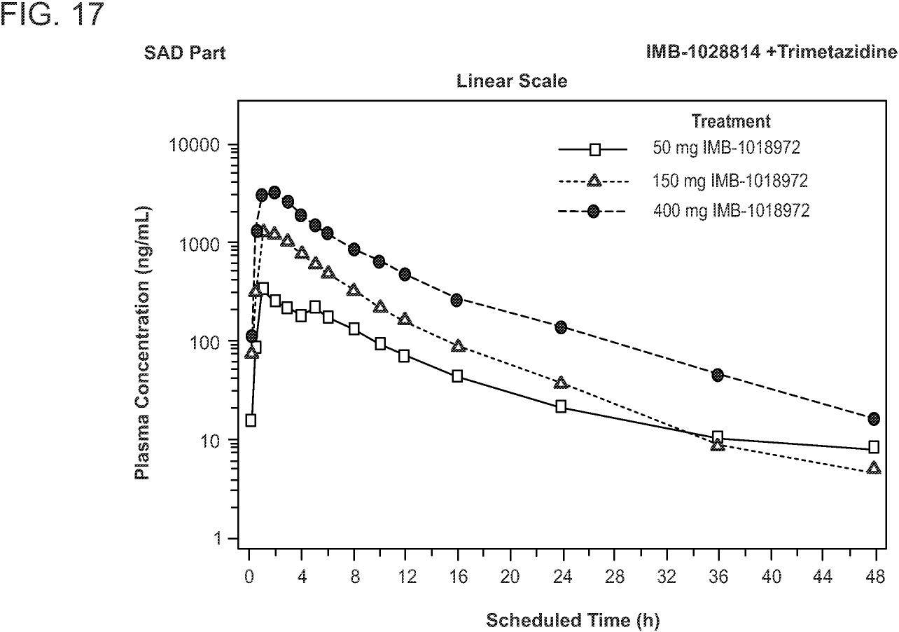

FIG. 16 is a graph of Geometric Mean IMB-1028814 + Trimetazidine Plasma Concentration-Time Profiles (Semi-Logarithmic) - SAD Part (PK Set)

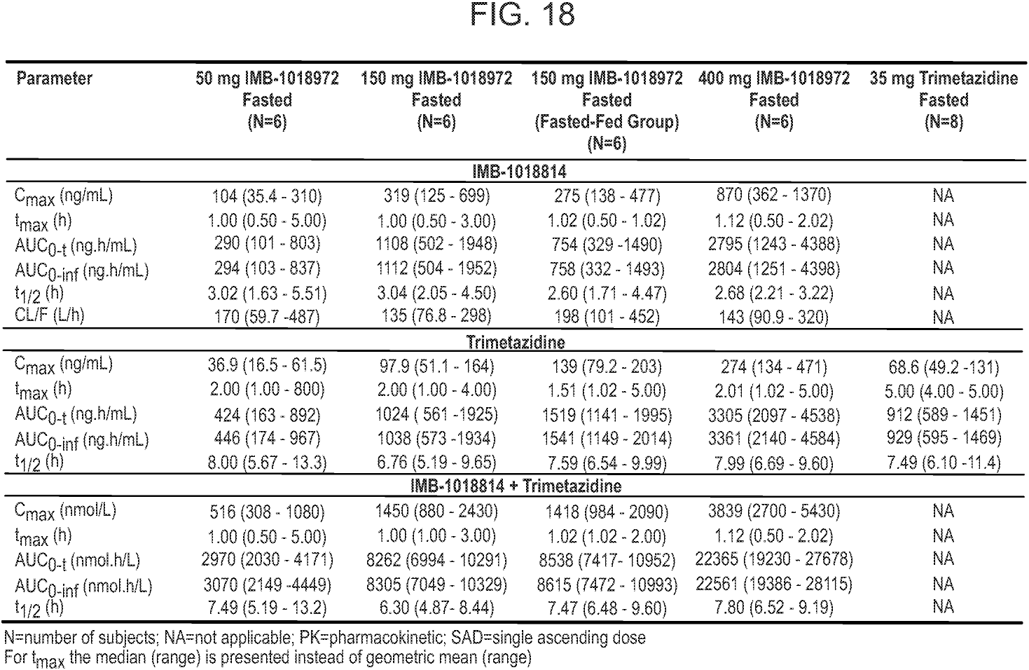

FIG. 17 is a graph of Geometric Mean IMB-1028814 + Trimetazidine Plasma Concentration-Time Profiles (Semi-Logarithmic) - SAD Part (PK Set)

FIG. 18 is a table of Summary Statistics (Geometric Mean [Range]) of IMB-1028814, Trimetazidine, and IMB-1028814 + Trimetazidine Plasma Pharmacokinetic Parameters - SAD Part (PK Set)

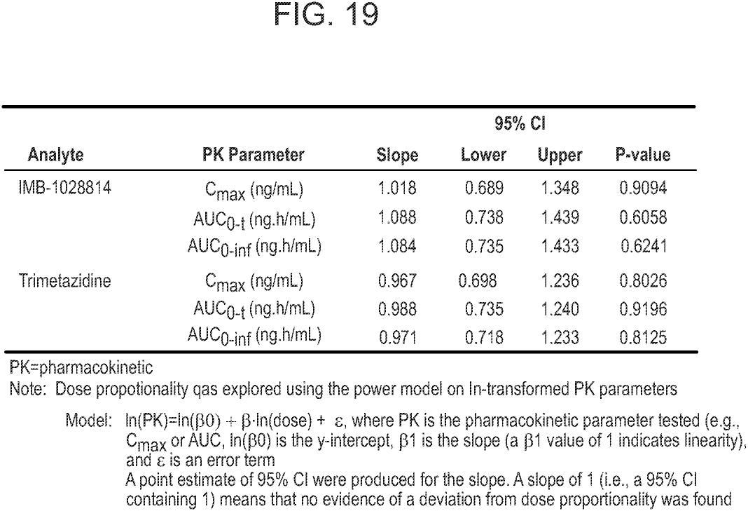

FIG. 19 is a table of Exploratory Analysis of Dose Proportionality for IMB-1028814 and Trimetazidine over the Dose Range of 50 mg to 400 mg IMB-1018972 under Faster Conditions -SAD Part (PK Set)

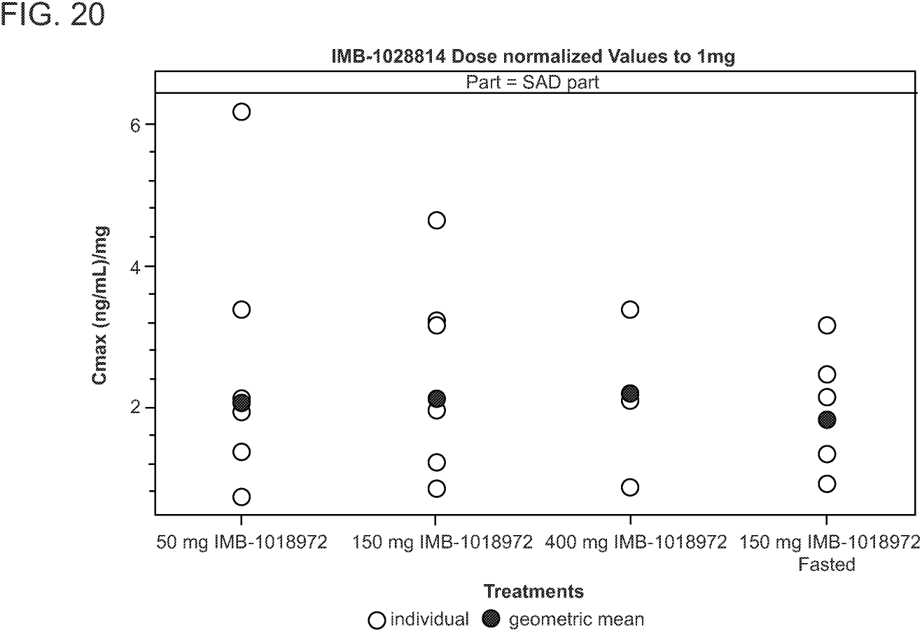

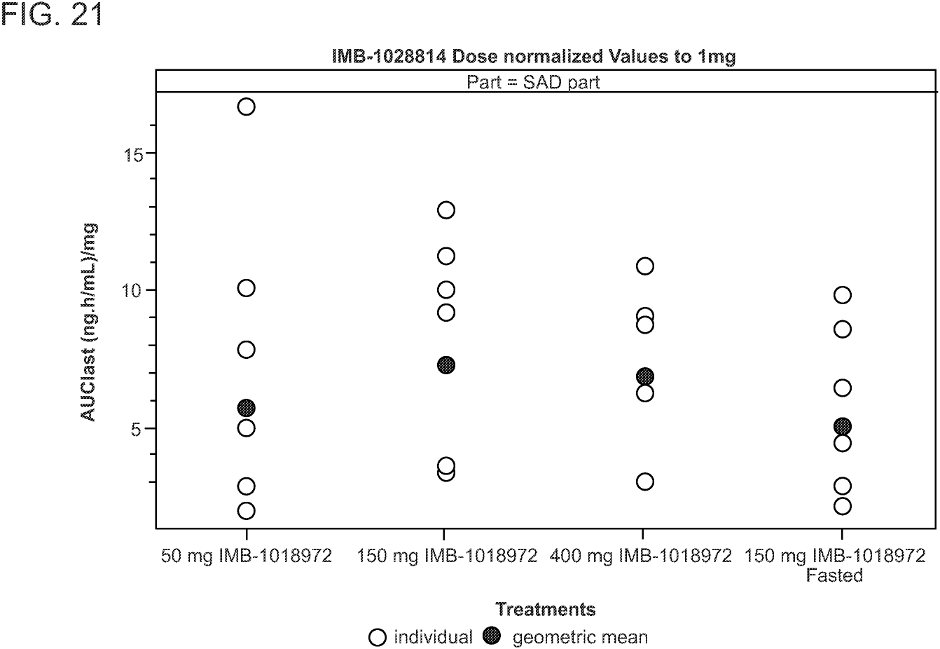

FIG. 20 is a graph of Plot of Combined Individual and Geometric Mean Dose-Normalized IMB-1028814 Cₘₐₓ over the Dose Range of 50 mg to 400 mg IMB-1018972 under Fasted Conditions - SAD Part (PK Set)

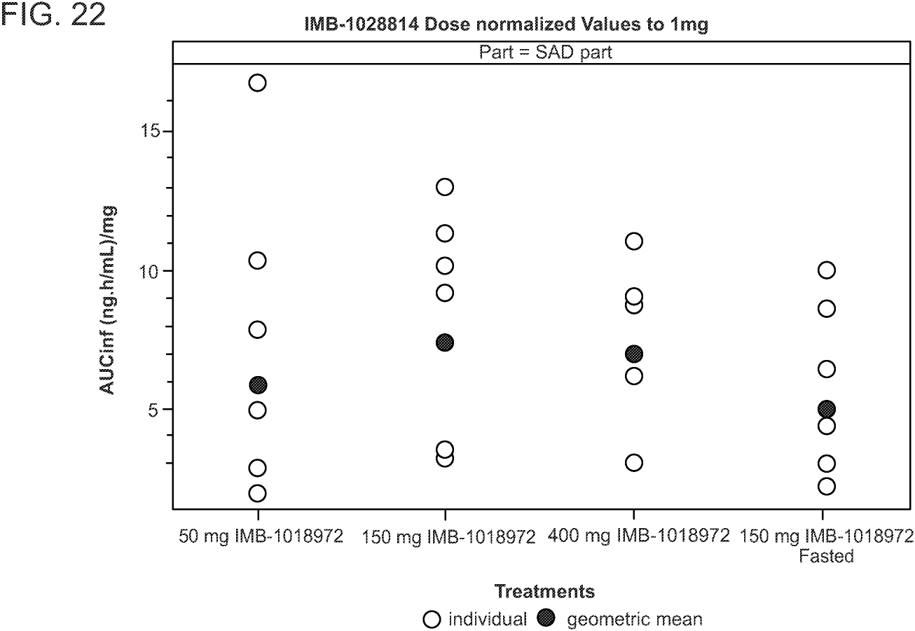

FIG. 21 is a graph of Plot of Combined Individual and Geometric Mean Dose-Normalized IMB-1028814 AUC₀₋ₜ over the Dose Range of 50 mg to 400 mg IMB-1018972 under Fasted Conditions - SAD Part (PK Set)

FIG. 22 is a graph of Plot of Combined Individual and Geometric Mean Dose-Normalized IMB-1028814 AUC_{0-inf} over the Dose Range of 50 mg to 400 mg IMB-1018972 under Fasted Conditions - SAD Part (PK Set)

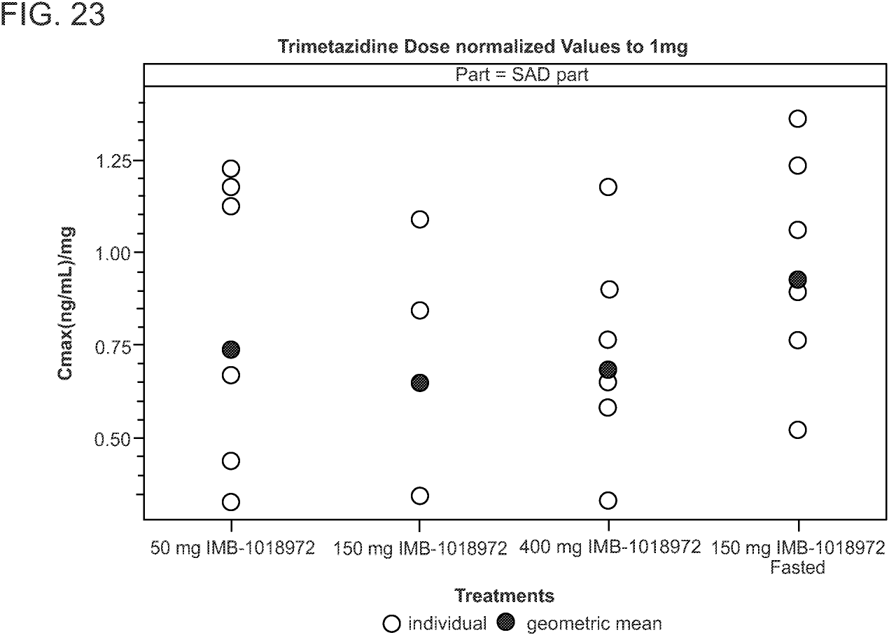

FIG. 23 is a graph of Plot of Combined Individual and Geometric Mean Dose-Normalized Trimetazidine Cₘₐₓ over the Dose Range of 50 mg to 400 mg IMB-1018972 under Fasted Conditions - SAD Part (PK Set)

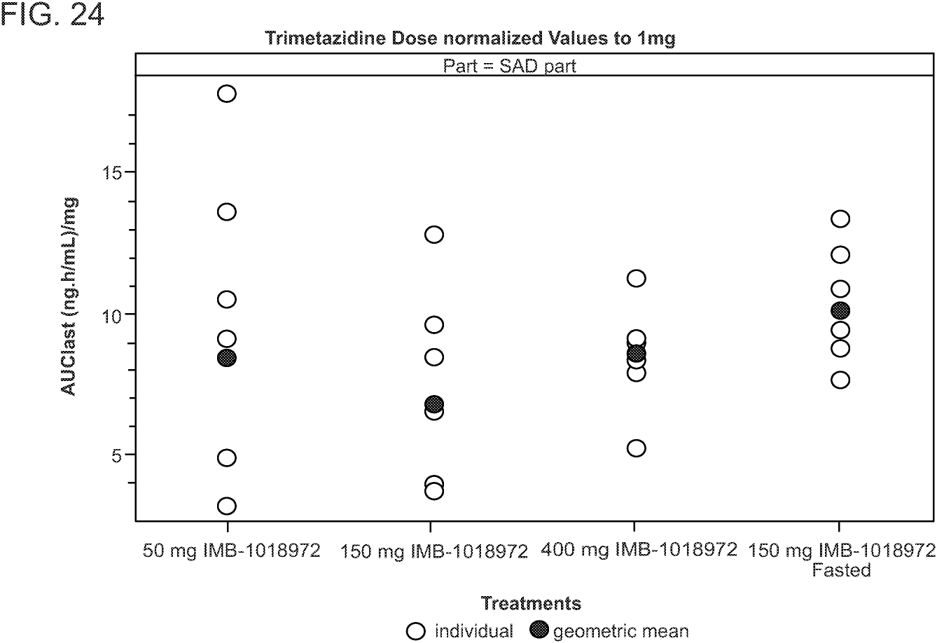

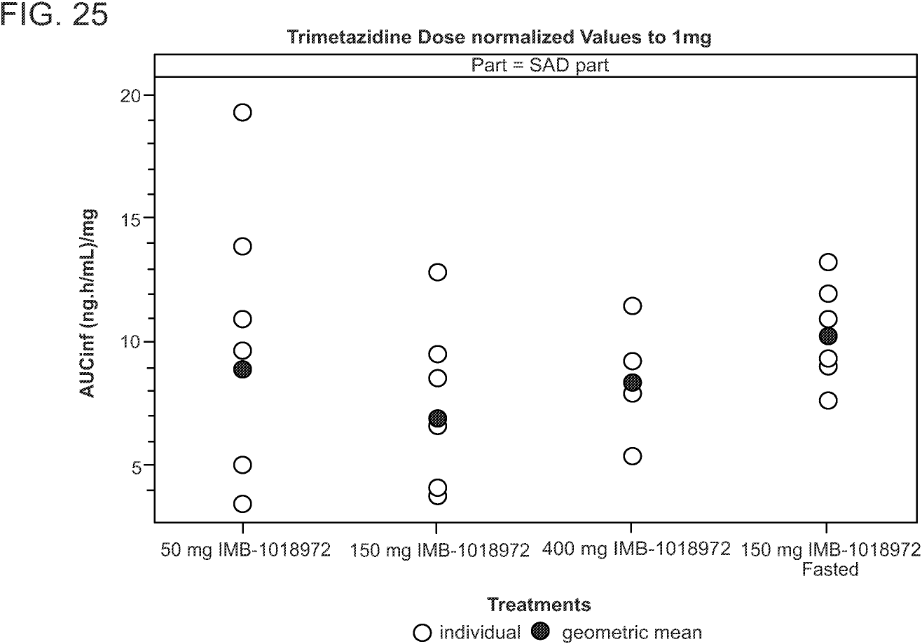

FIG. 24 is a graph of Plot of Combined Individual and Geometric Mean Dose-Normalized Trimetazidine AUC₀₋ₜ over the Dose Range of 50 mg to 400 mg IMB-1018972 under Fasted Conditions - SAD Part (PK Set)

FIG. 25 is a graph of Plot of Combined Individual and Geometric Mean Dose-Normalized Trimetazidine AUC_{0-inf} over the Dose Range of 50 mg to 400 mg IMB-1018972 under Fasted Conditions - SAD Part (PK Set)

### Effect of Food

The possible effect of food on the PK of IMB-1028814 and trimetazidine was explored by comparing administration of single oral doses of 150 mg IMB-1018972 after an FDA-defined high-fat breakfast and under fasted conditions.

All predose samples were below the LLOQ for IMB-1028814 and trimetazidine plasma concentrations.

After study drug administration under fed conditions, the geometric mean IMB-1028814 plasma concentrations initially increased at the same speed as under fasted conditions but then a plateau was reached. When looking at individual profiles, no plateau was observed, but individual IMB-1028814 tmax values ranged between 0.42 and 5 hours. Median tmax was reached at 2 hourspostdose under fed conditions relative to 1 hour postdose under fasted conditions.

The trimetazidine plasma concentrations under fed conditions increased less rapidly than after study drug administration under fasted conditions and median tmax was reached at 4 hours postdose under fed conditions relative to 1.5 hours postdose under fasted conditions.

No evidence for an effect of food was observed on the IMB-1028814 exposure parameters AUC₀₋ₜ and AUC_{0-inf} (both with an estimate of 1.12 and 90% CI ranging from 1.02 to 1.23). However, Cmax was approximately 36% lower following administration of a single dose of 150 mg IMB-1018972 after an FDA-defined high-fat breakfast relative to administration under fasted conditions (estimate of 0.64; 90% CI ranging from 0.39 to 1.04).

No evidence for an effect of food was observed on the trimetazidine exposure parameters Cmax (estimate of 0.91; 90% CI ranging from 0.85 to 0.98), and AUC₀₋ₜ and AUC_{0-inf} (both with an estimate of 1.04 and 90% CI ranging from 0.98 to 1.10).

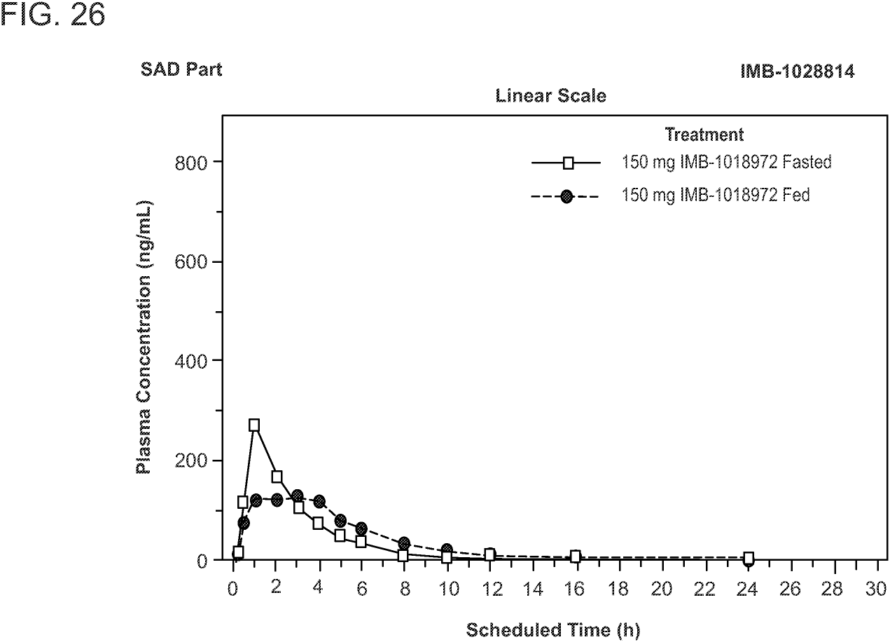

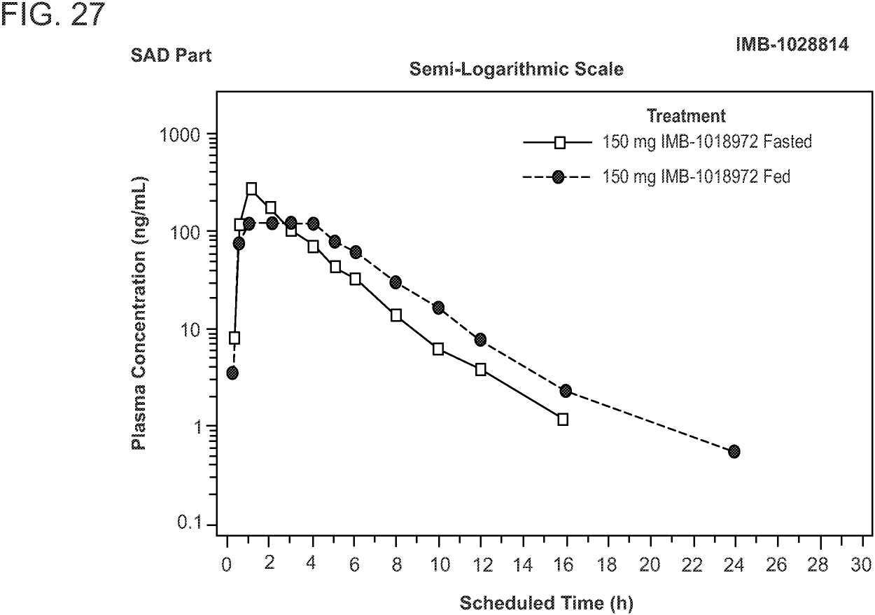

FIG. 26 is a graph of Geometric Mean IMB-1028814 Plasma Concentration-Time Profiles (Linear) - FE Arm of SAD Part (PK Set)

FIG. 27 is a graph of Geometric Mean IMB-1028814 Plasma Concentration-Time Profiles (Semi-Logarithmic Scale) - FE Arm of SAD Part (PK Set)

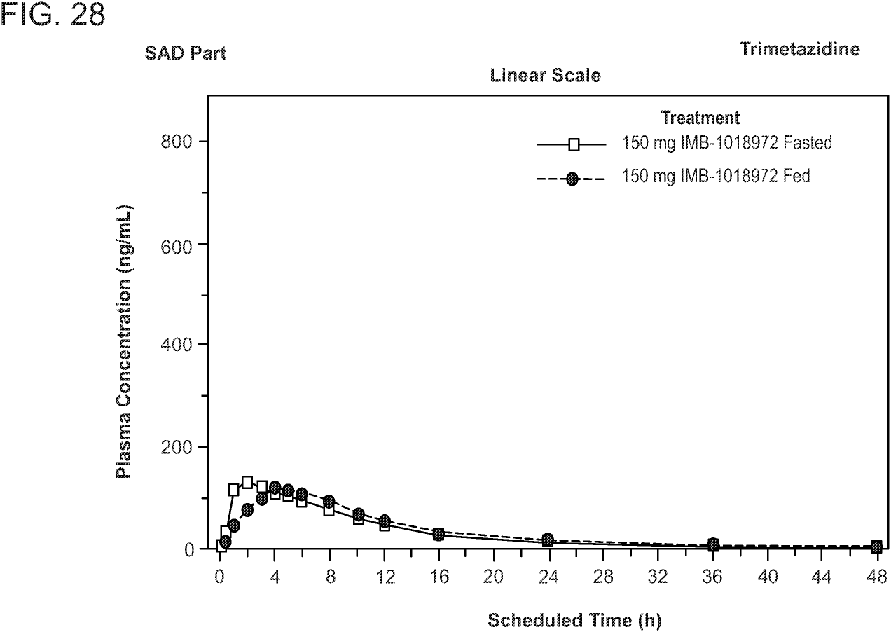

FIG. 28 is a graph of Geometric Mean Trimetazidine Plasma Concentration-Time Profiles (Linear) - FE Arm of SAD Part (PK Set)

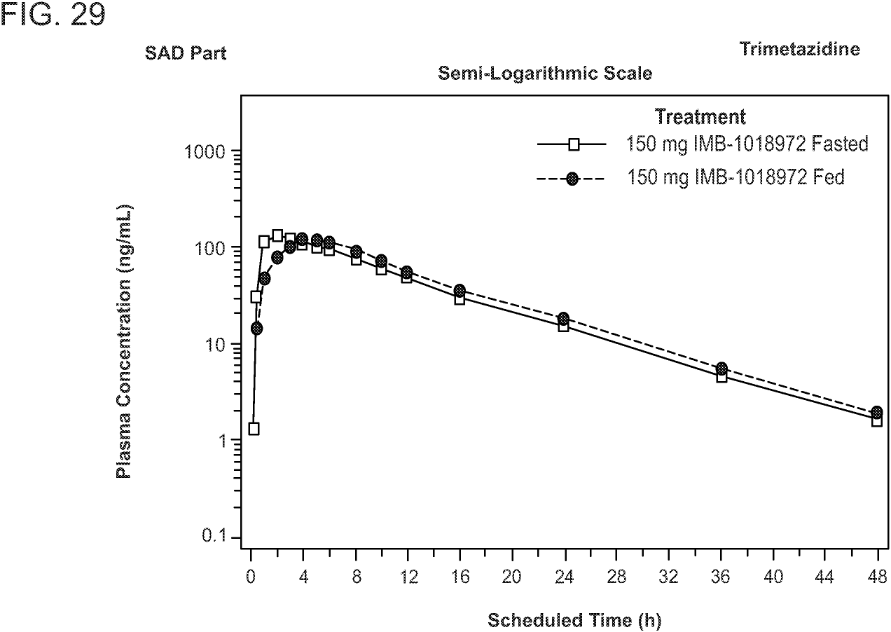

FIG. 29 is a graph of Geometric Mean Trimetazidine Plasma Concentration-Time Profiles (Semi-Logarithmic Scale) - FE Arm of SAD Part (PK Set)

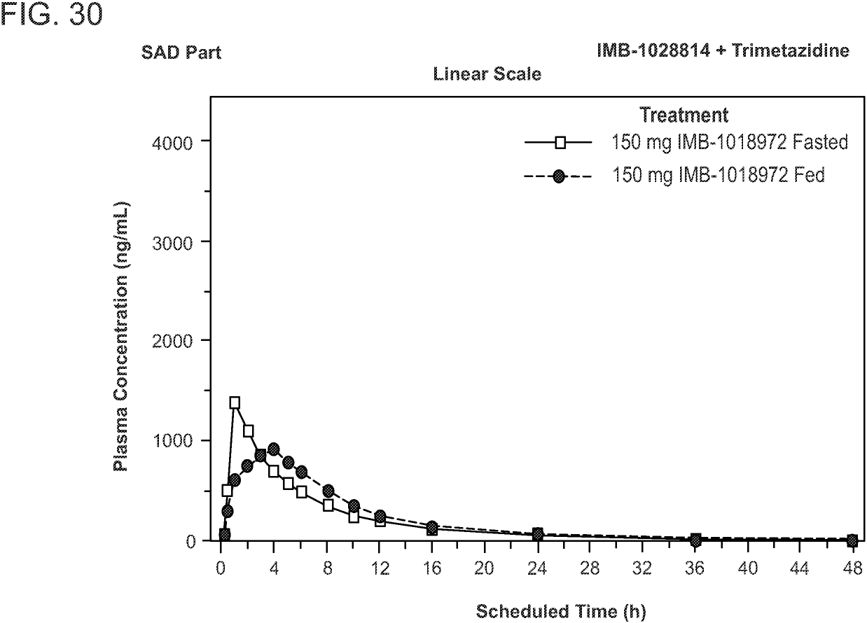

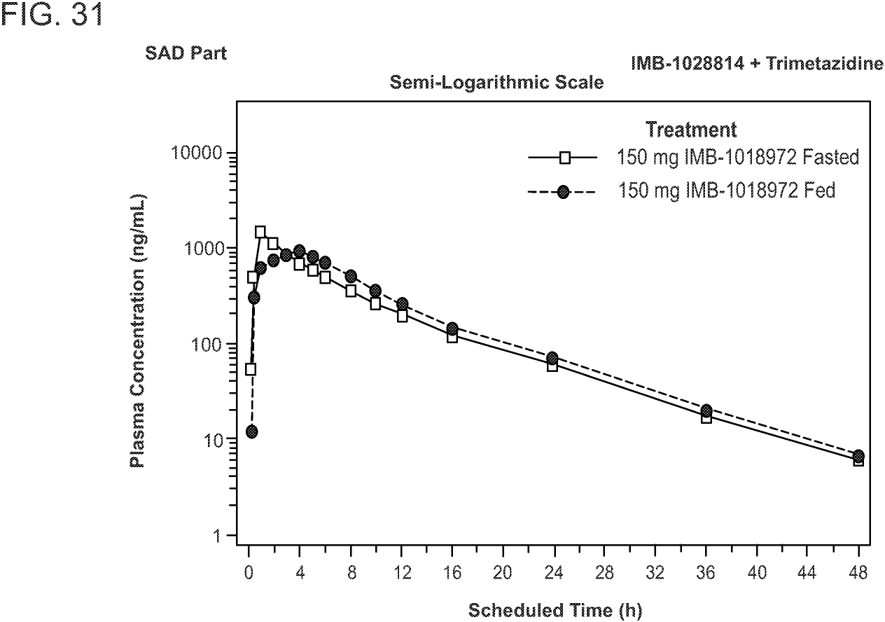

FIG. 30 is a graph of Geometric Mean IMB-1028814 + Trimetazidine Plasma Concentration-Time Profiles (Linear) - FE Arm of SAD Part (PK Set)

FIG. 31 is a graph of Geometric Mean IMB-1028814 + Trimetazidine Plasma Concentration-Time Profiles (Semi-Logarithmic Scale) - FE Arm of SAD Part (PK Set)

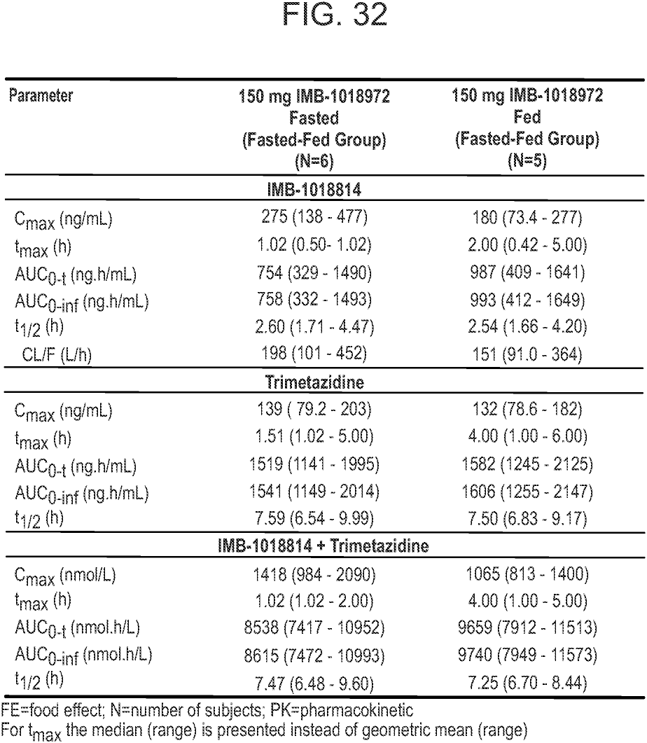

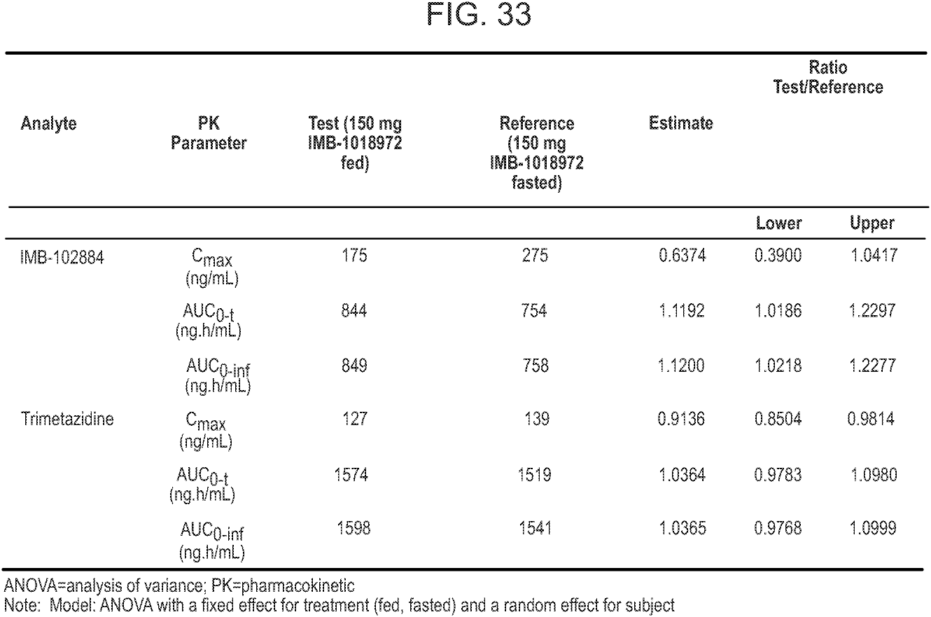

FIG. 32 is a table of Summary Statistics (Geometric Mean [Range]) of IMB-1028814, Trimetazidine, and IMB-128814 + Trimetazidine, and IMB-1028814 + Trimetazidine Plasma Pharmacokinetic Parameters - FE Arm of SAD Part (PK Set)

FIG. 33 is a table of Exploratory Analysis of Food Effect for IMB-1028814 and Trimetazidine following Administration of 150 mg IMB-1018972 - FE Arm of SAD Part (PK Set)

### Pharmacokinetic Results of IMB-1028814 and Trimetazidine in Urine

Urinary excretion of IMB-1028814 and trimetazidine was determined in urine samples from subjects who received a single oral dose of IMB-1018972 in the range of 50 mg to 400 mg under fasted conditions. Further, urinary excretion of trimetazidine was determined in urine samples from subjects who received a single oral dose of 35 mg trimetazidine.

The arithmetic mean percent of the dose excreted in urine ranged between 3.99% and 5.74% for IMB-1028814, and between 23.11% and 32.55% for trimetazidine within 48 hours after a single oral IMB-1018972 dose over the studied dose range of 50 mg to 400 mg. Within 48 hours following administration of a single oral dose of 35 mg trimetazidine, an arithmetic mean of 54.47% was excreted in urine as trimetazidine. These results indicate that metabolism is the primary clearance mechanism for IMB-1028814 while renal excretion is the primary clearance mechanism for trimetazidine.

The geometric mean renal clearance (CLR) ranged between 3.76 L/h and 5.37 L/h for IMB-1028814, and between 18.1 L/h and 20.8 L/h for trimetazidine over the studied single-dose range of 50 mg to 400 mg IMB-1018972. Geometric mean CLR for trimetazidine was 20.4 L/h following administration of a single oral dose of 35 mg trimetazidine. The renal clearance of trimetazidine is greater than glomerular filtration rate (125 mL/min or 7.5 L/h), indicating that trimetazidine undergoes net tubular secretion.

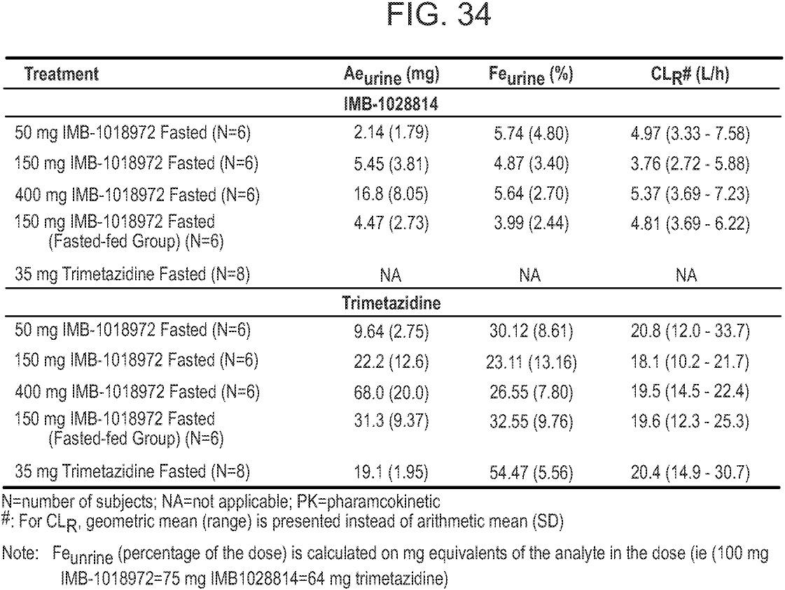

FIG. 34 is a table of Summary Statistics (Arithmetic Mean [SD]) of Urine Pharmacokinetic Parameters for IMB-1028814, Trimetazidine, and IMB-1028814 + Trimetazidine - SAD Part (PK Set)

### Pharmacokinetic Results of IMB-1028814 and Trimetazidine in Urine

Urinary excretion of IMB-1028814 and trimetazidine was determined in urine samples from subjects who received a single oral dose of IMB-1018972 in the range of 50 mg to 400 mg under fasted conditions. Further, urinary excretion of trimetazidine was determined in urine samples from subjects who received a single oral dose of 35 mg trimetazidine.

The arithmetic mean percent of the dose excreted in urine ranged between 3.99% and 5.74% for IMB-1028814, and between 23.11% and 32.55% for trimetazidine within 48 hours after a single oral IMB-1018972 dose over the studied dose range of 50 mg to 400 mg. Within 48 hours following administration of a single oral dose of 35 mg trimetazidine, an arithmetic mean of 54.47% was excreted in urine as trimetazidine. These results indicate that metabolism is the primary clearance mechanism for IMB-1028814 while renal excretion is the primary clearance mechanism for trimetazidine.

The geometric mean renal clearance (CLR) ranged between 3.76 L/h and 5.37 L/h for IMB-1028814, and between 18.1 L/h and 20.8 L/h for trimetazidine over the studied single-dose range of 50 mg to 400 mg IMB-1018972. Geometric mean CLR for trimetazidine was 20.4 L/h following administration of a single oral dose of 35 mg trimetazidine. The renal clearance of trimetazidine is greater than glomerular filtration rate (125 mL/min or 7.5 L/h), indicating that trimetazidine undergoes net tubular secretion.

FIG. 33 is a table of Summary Statistics (Arithmetic Mean [SD]) of Urine Pharmacokinetic Parameters for IMB-1028814, Trimetazidine, and IMB-1028814 + Trimetazidine - SAD Part (PK Set)

### MAD Part

### Pharmacokinetic Results of IMB-1028814, Trimetazidine and IMB-1028814 + Trimetazidine in Plasma

All predose samples on Day 1 were below the LLOQ for IMB-1028814 and trimetazidine plasma concentrations.

The geometric mean concentration-time profiles for IMB-1028814, metabolite trimetazidine, and IMB-1028814 + trimetazidine on Day 1 and Day 14 showed a dose dependent increase in plasma concentrations following administration of multiple doses of IMB-1018972 under fed conditions of 50 mg q12h and 150 mg q12h.

Similar to the SAD part, initial hydrolysis of IMB-1018972 to IMB-1028814 and subsequent systemic bioavailability of IMB-1028814 on Days 1 and 14 was relatively rapid. Over the 2 multiple-dose levels, median tmax ranged between 0.5 hours and 1 hours postdose for IMB-1028814 on Day 1, and was 3 hours postdose for trimetazidine on Day 1. On Day 14, median tmax was 0.5 hours postdose for IMB-1028814 and 2 hours postdose for trimetazidine.

### Exposure Parameters on Day 1

No dose-proportionality analysis was done since there were only 2 IMB-1018972 dose levels in the MAD part: multiple oral doses of 50 mg or 150 mg q12h for 14 days under fed conditions.

The geometric mean Cₘₐₓ and AUC_{0-T} were higher after 150 mg fed than after 50 mg fed for IMB-1028814 (297% and 336% higher for Cₘₐₓ and AUC_{0-T}, respectively), trimetazidine (154% and 163% higher for Cₘₐₓ and AUC_{0-T}, respectively), and IMB-1028814 + trimetazidine (257% and 239% higher for Cmax and AUC_{0-T}, respectively).

When comparing the MAD and SAD parts, geometric mean Cmax was 97% higher on Day 1 after 150 mg fed in the MAD part than after a single dose of 150 mg fed in the SAD part for IMB-1028814. For trimetazidine however, geometric mean Cmax was 32% lower on Day 1 after 150 mg fed in the MAD part than after a single dose of 150 mg fed in the SAD part.

### Exposure Parameters on Day 14 Following Repeated Q12h Dosing

The geometric mean Cmax and AUC_{0-T} were higher after 150 mg fed than after 50 mg fed for IMB-1028814 (377% and 367% higher for Cmax and AUC_{0-T}, respectively), trimetazidine (127% and 126% higher for Cmax and AUC_{0-T}, respectively), and IMB-1028814 + trimetazidine (286% and 211% higher for Cmax and AUC_{0-T}, respectively).

The predefined stopping criterion for IMB-1028814 plasma exposure of 417,733 and 652,849 ng.h/mL for males and females, respectively, was not reached by any of the subjects during the MAD part.

### Trough Concentrations Following Repeated Q12h Dosing

Based upon visual inspection of the geometric mean plasma concentration-time profiles and the geometric mean trough concentrations, it can be concluded that for both 150 mg fed and 50 mg fed, the Day 14 IMB-1018972 dose was administered under steady-state conditions of IMB-1028814 and trimetazidine concentrations

### Accumulation Following Repeated Q12h Dosing

For both the 50 mg and 150 mg fed dose levels, geometric mean AUC_{0-T} values of IMB-1028814, trimetazidine, and IMB-1028814 + trimetazidine were higher on Day 14 relative to Day 1.

Geometric mean R_{ac} for IMB-1028814 was 1.18 and 1.10 after the 150 mg fed dose and 50 mg fed dose, respectively, indicating minimal accumulation of IMB-1028814 in plasma. Geometric mean R_{ac} for trimetazidine was 1.63 and 1.89 after the 150 mg fed dose and 50 mg fed dose, respectively, indicating modest accumulation of trimetazidine in plasma. Geometric mean R_{ac} for IMB-1028814 + trimetazidine was 1.39 and 1.52 after the 150 mg fed dose and 50 mg fed dose, respectively, indicating modest accumulation of IMB-1028814 + trimetazidine in plasma.

### Terminal Elimination Half-Life Following Repeated Q12h Dosing

For IMB-1028814, the geometric mean t1/2 of 4.48 hours after 150 mg fed was longer than that of 2.79 hours after 50 mg fed. For trimetazidine, the geometric mean t1/2 of 9.36 hours after 150 mg fed was similar to that of 9.32 hours after 50 mg fed. For IMB-1028814 + trimetazidine, the geometric mean t1/2 of 8.90 hours for IMB-1028814 after 150 mg fed was similar to that of 9.08 hours after 50 mg fed.

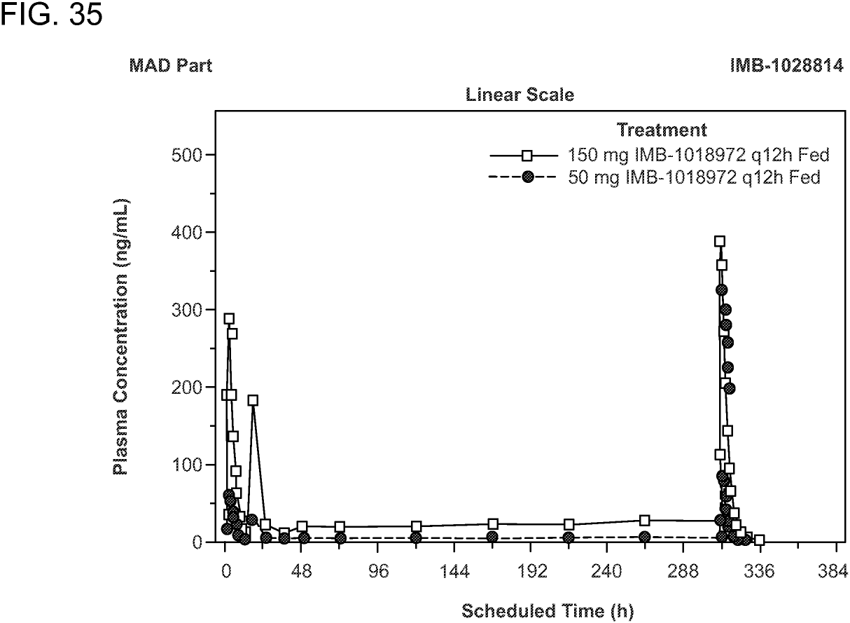

FIG. 35 is a graph of Geometric Mean IMB-1028814 Plasma Concentration-Time Profiles from Day 1 through Day 14 (Linear) - MAD Part (PK Set)

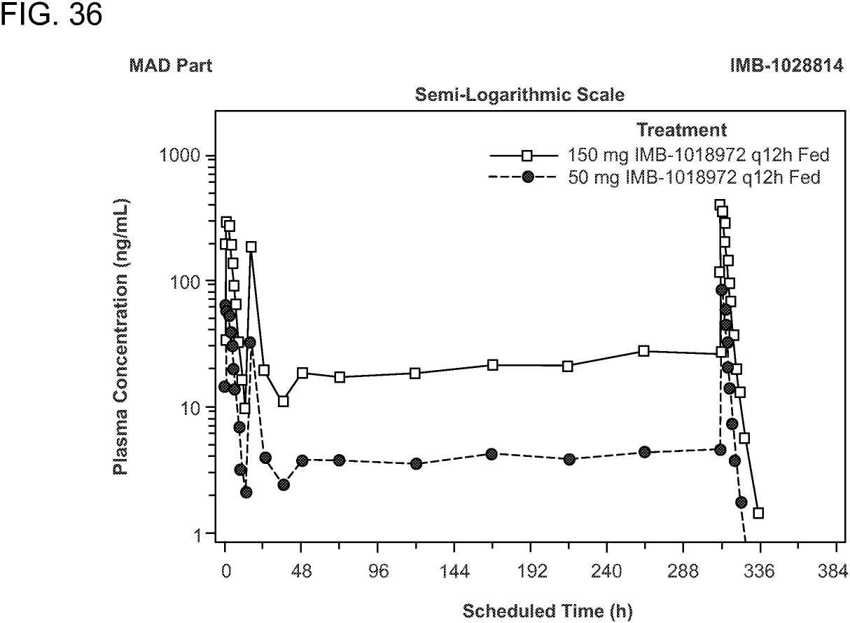

FIG. 36 is a graph of Geometric Mean IMB-1028814 Plasma Concentration-Time Profiles from Day 1 through Day 14 (Semi-Logarithmic Scale) - MAD Part (PK Set)

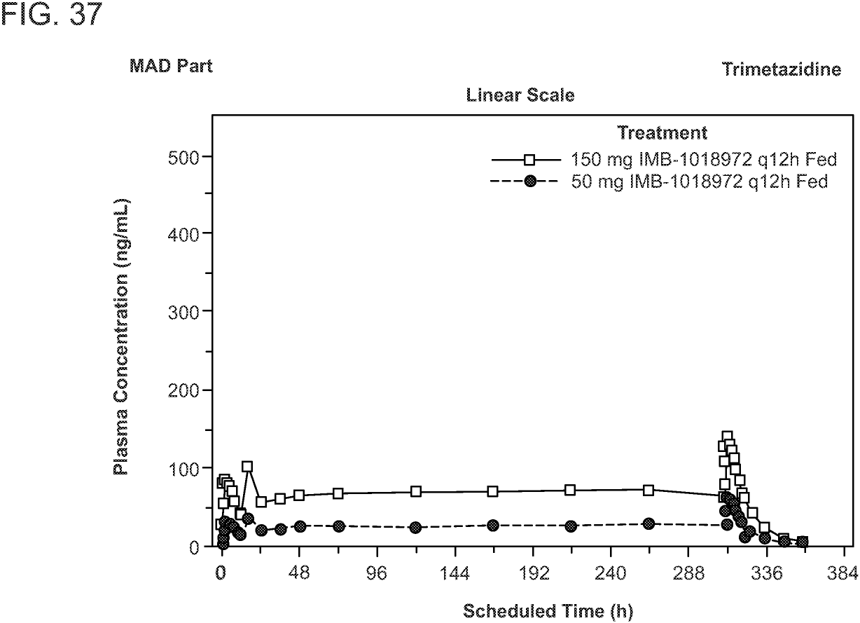

FIG. 37 is a graph of Geometric Mean Trimetazidine Plasma Concentration-Time Profiles from Day 1 through Day 14 (Linear) - MAD Part (PK Set)

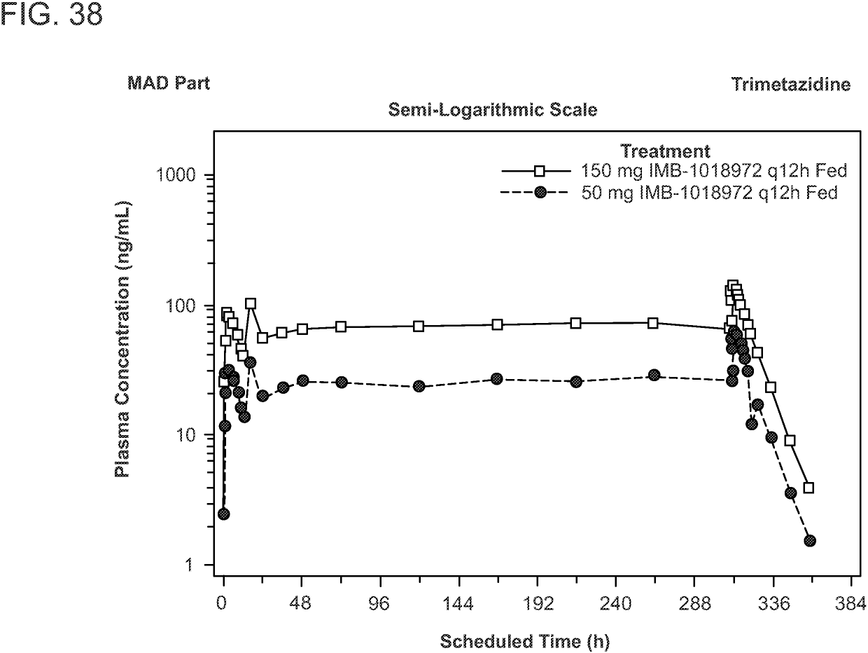

FIG. 38 is a graph of Geometric Mean Trimetazidine Plasma Concentration-Time Profiles from Day 1 through Day 14 (Semi-Logarithmic Scale) - MAD Part (PK Set)

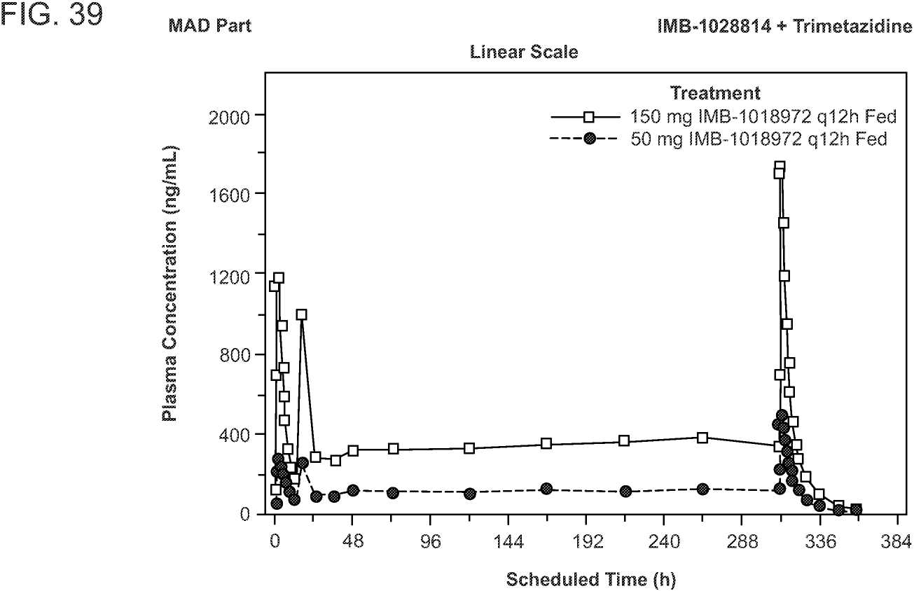

FIG. 39 is a graph of Geometric Mean IMB-1028814 + Trimetazidine Plasma Concentration-Time Profiles from Day 1 through Day 14 (Linear) - MAD Part (PK Set)

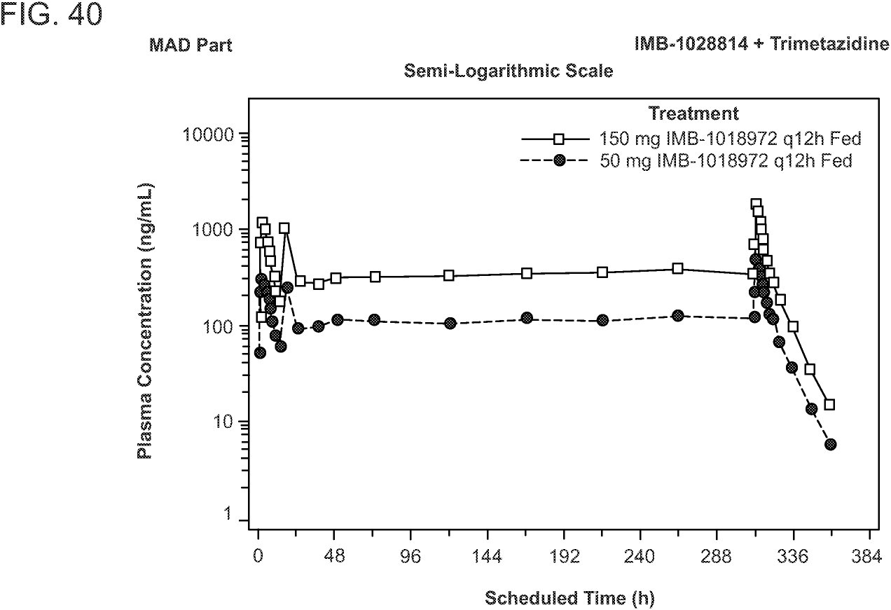

FIG. 40 is a graph of Geometric Mean IMB-1028814 + Trimetazidine Plasma Concentration-Time Profiles from Day 1 through Day 14 (Semi-Logarithmic Scale) - MAD Part (PK Set)

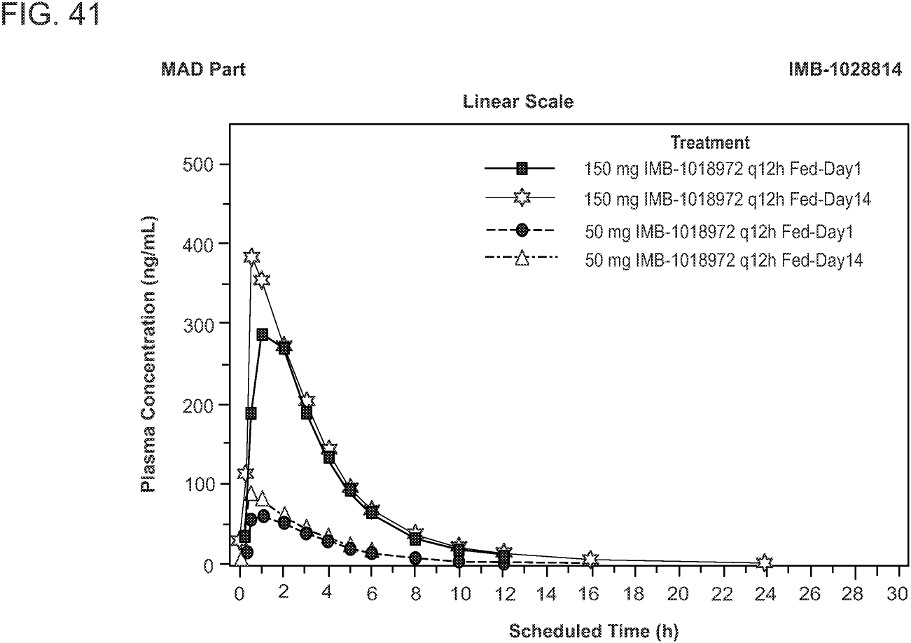

FIG. 41 is a graph of Geometric Mean IMB-1028814 Plasma Concentration-Time Profiles from Day 1 through Day 14 (Linear) - MAD Part (PK Set)

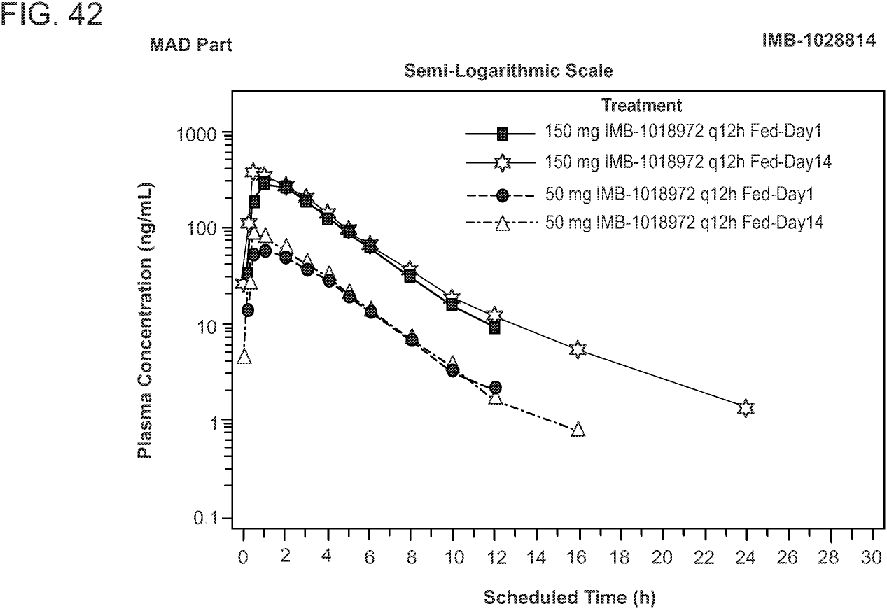

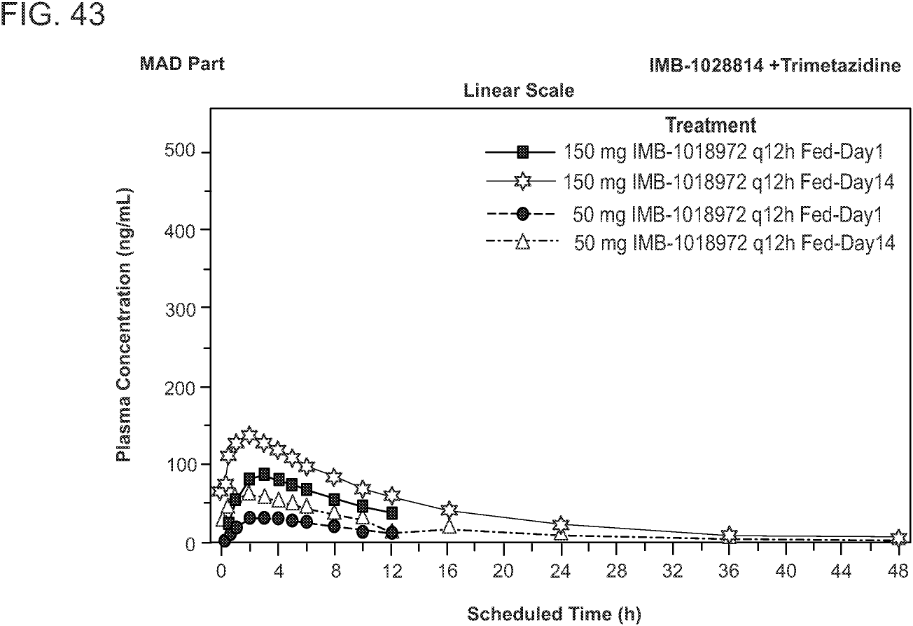

FIG. 42 is a graph of Geometric Mean IMB-1028814 Plasma Concentration-Time Profiles from Day 1 through Day 14 (Semi-Logarithmic Scale) - MAD Part (PK Set)

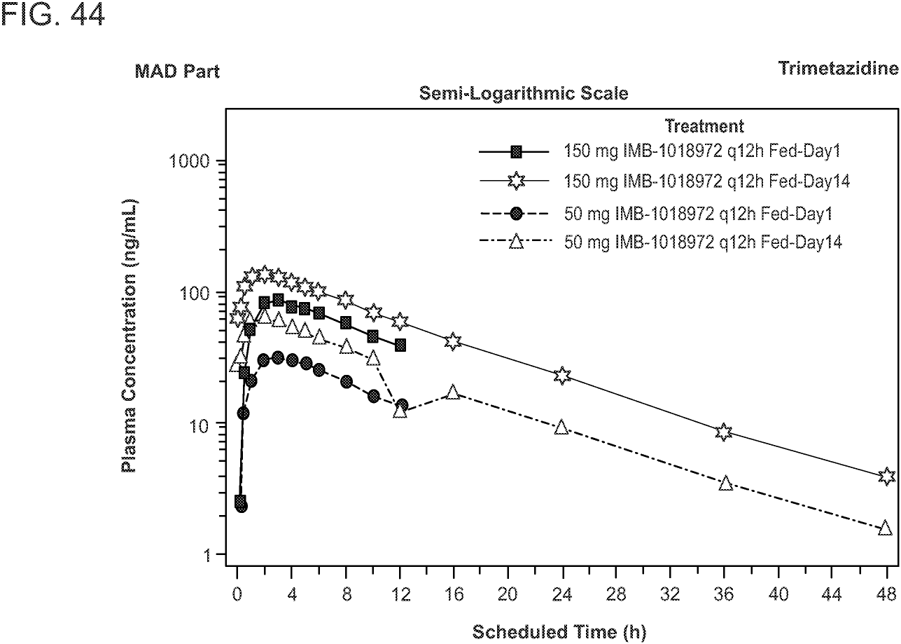

FIG. 43 is a graph of Geometric Mean Trimetazidine Plasma Concentration-Time Profiles after Dosing on Day 1 through Day 14 (Linear) - MAD Part (PK Set)

FIG. 44 is a graph of Geometric Mean Trimetazidine Plasma Concentration-Time Profiles after Dosing on Day 1 through Day 14 (Semi-Logarithmic Scale) - MAD Part (PK Set)

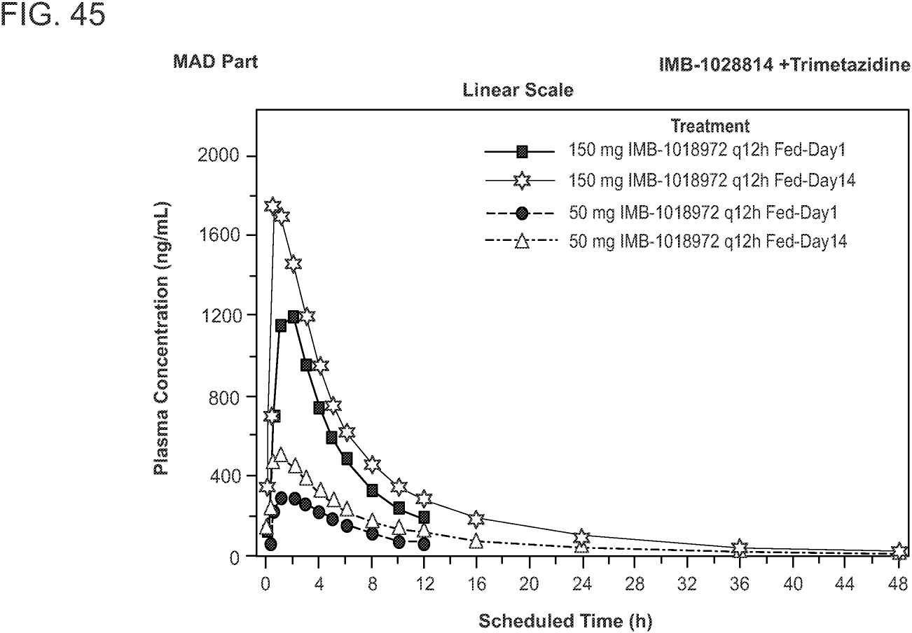

FIG. 45 is a graph of Geometric Mean IMB-1028814 + Trimetazidine Plasma Concentration-Time Profiles after Dosing on Day 1 through Day 14 (Linear) - MAD Part (PK Set)

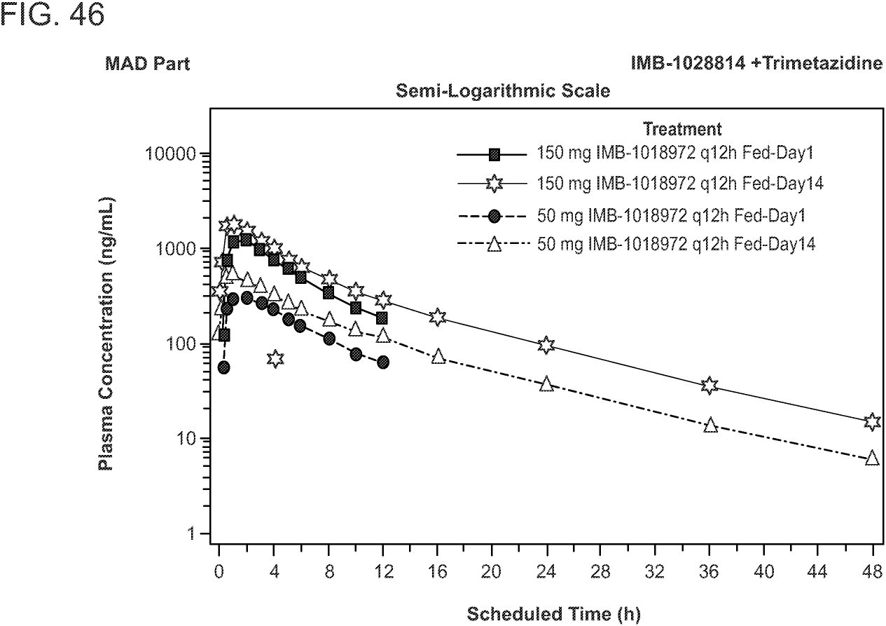

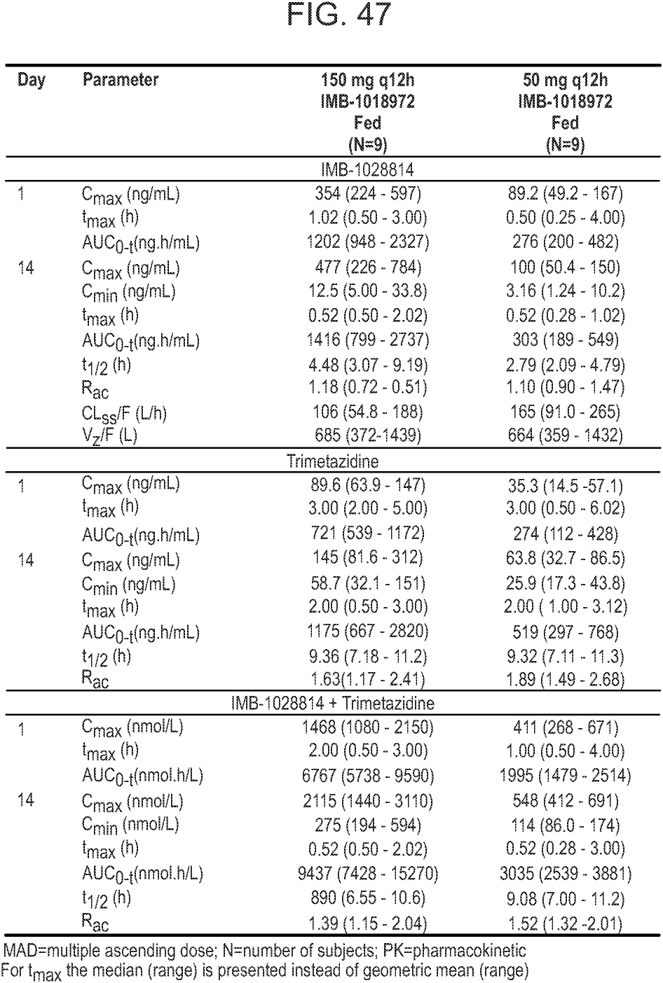

FIG. 46 is a graph of Geometric Mean IMB-1028814 + Trimetazidine Plasma Concentration-Time Profiles after Dosing on Day 1 through Day 14 (Semi-Logarithmic Scale) -MAD Part (PK Set)

FIG. 47 is a table of Summary Statistics (Geometric Mean [Range]) of IMB-1028814, Trimetazidine, and IMB-1028814 + Trimetazidine Plasma Pharmacokinetic Parameters - MAD Part (PK Set)

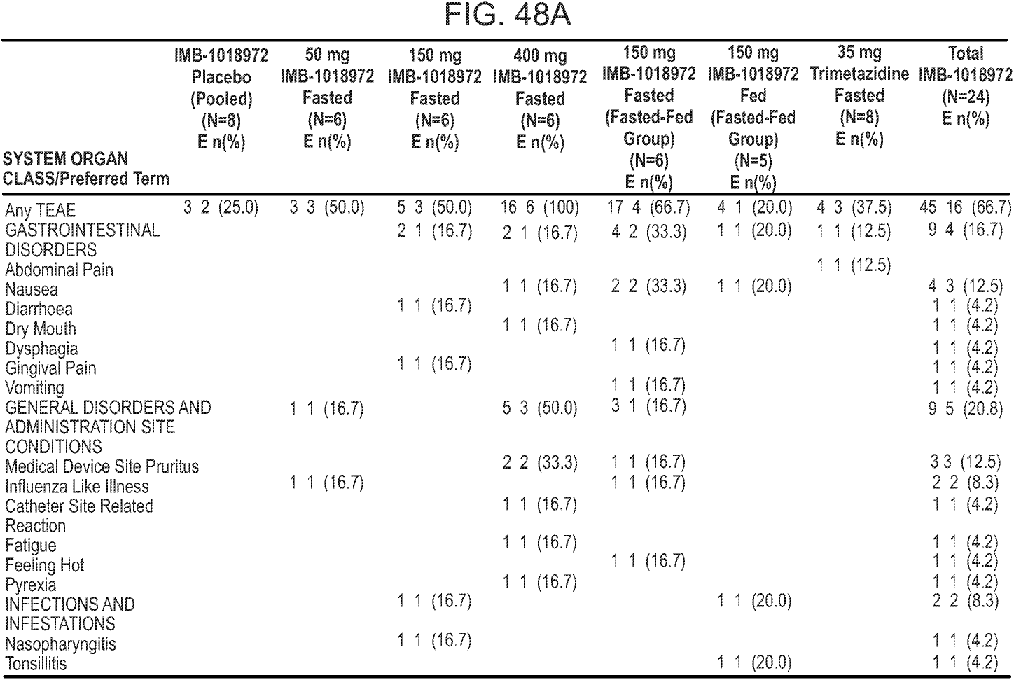

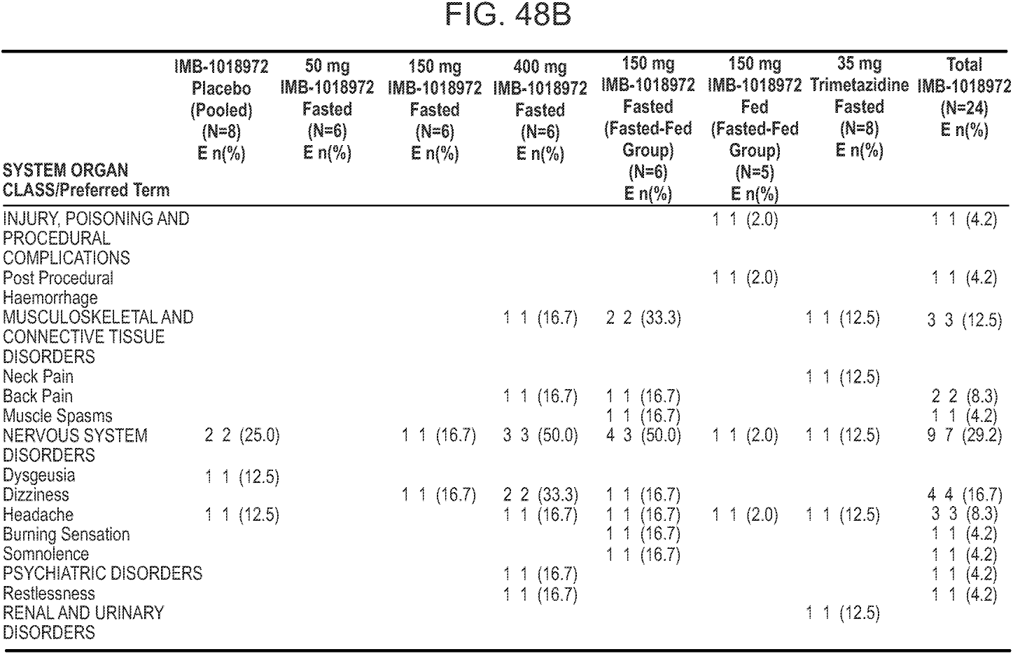

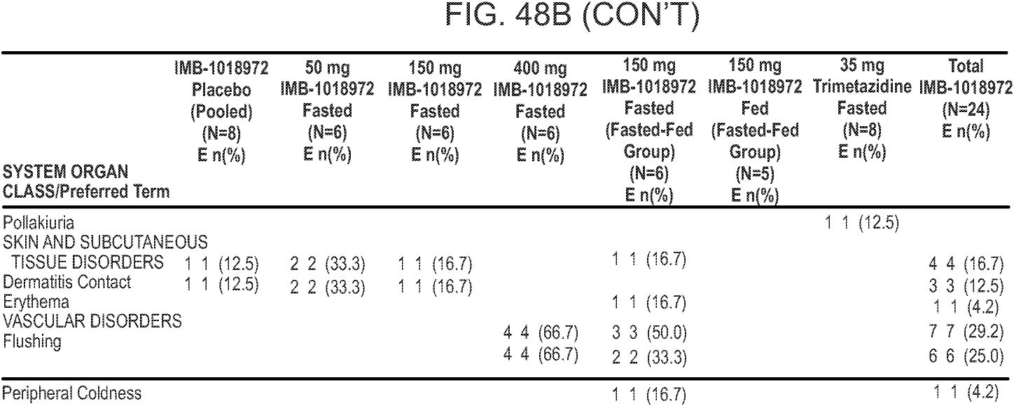

FIG. 48A and FIG. 48B is a table Summary of All TEAEs by System Organ Class, Preferred Term and Treatment - SAD Part (and integrated FE Arm) (Safety Set) with the following notifications:

%=number of subjects (n) as a percentage of number of subjects (N) per treatment; AE=adverse event; E=number of AEs; FE=food effect; MedDRA=Medical Dictionary for Regulatory Activities; N=number of subjects exposed; n=number of subjects that experienced the AE; SAD=single ascending dose; TEAE=treatment-emergent adverse event

Adverse events were classified according to MedDRA 22.0

Subjects were counted once, per preferred term, for multiple occurrences of a specific MedDRA term

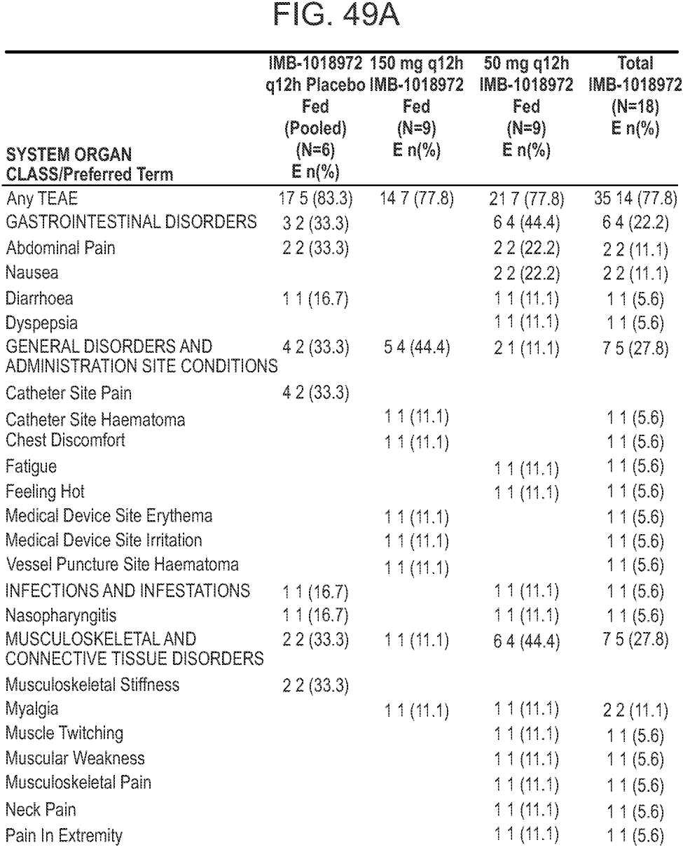

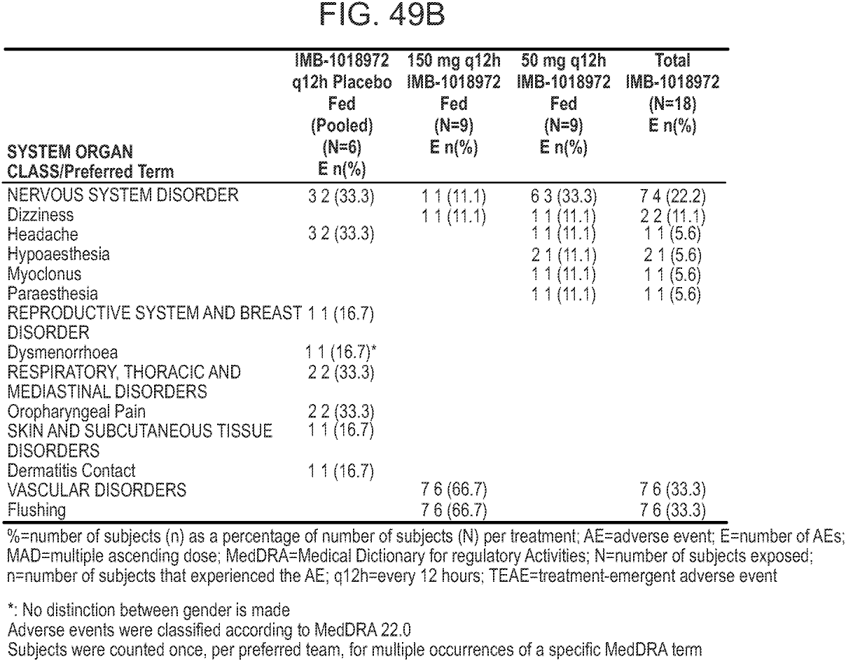

FIG. 49A and FIG. 49B is a table Summary of All TEAEs by System Organ Class, Preferred Term and Treatment - MAD Part (Safety Set)

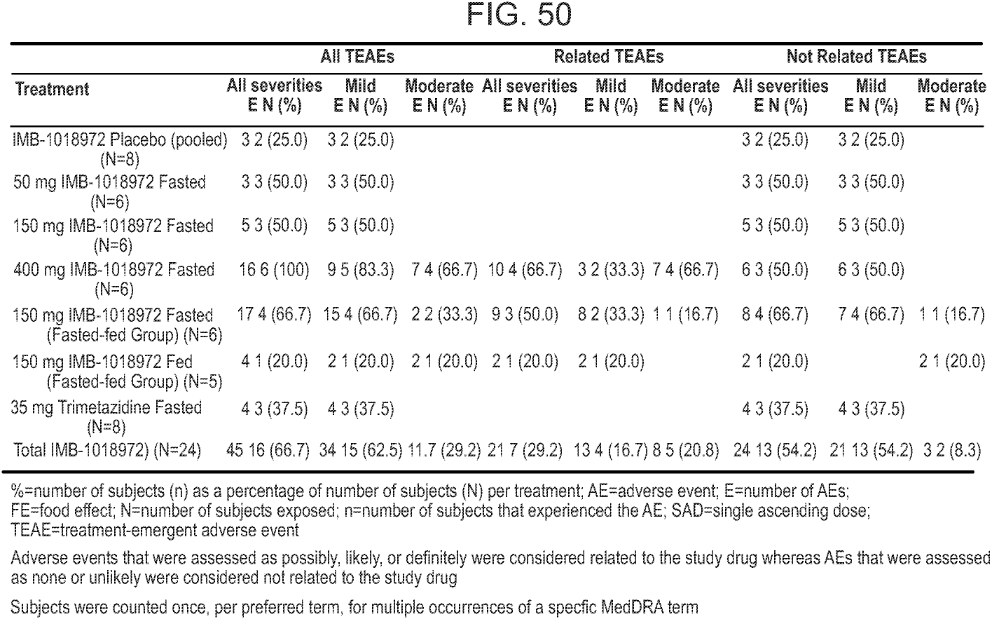

FIG. 50 is a table Summary of All TEAEs by Treatment, Relationship, and Severity-SAD Part (and Integrated FE Arm) (Safety Set)

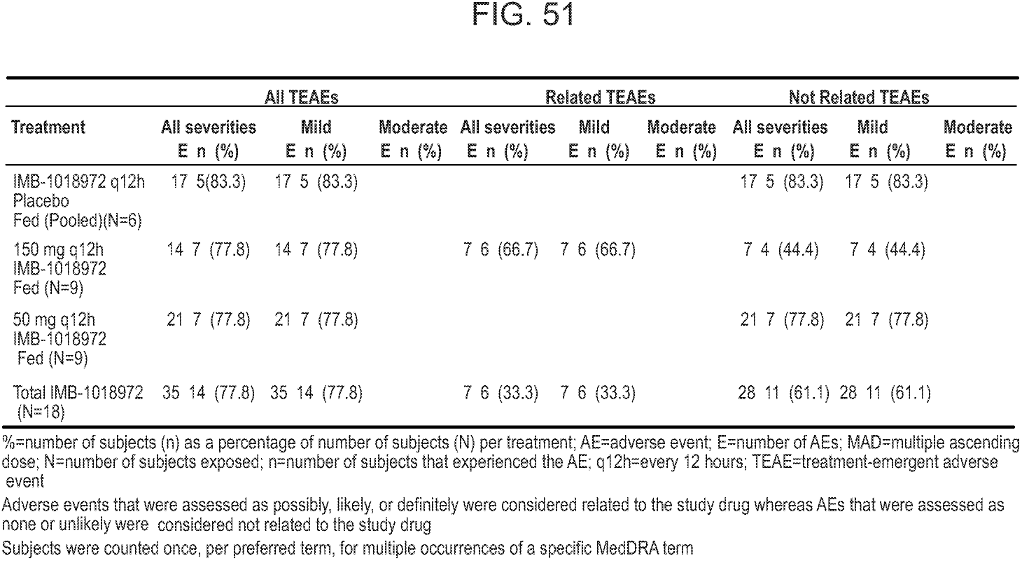

FIG. 51 is a table Summary of All TEAEs by Treatment, Relationship, and Severity -MAD Part (Safety Set)

### Summary of Adverse Events

### SAD Part (and Integrated FE Arm)

### TEAEs Reported with Administration of IMB-1018972 or Placebo

A total of 45 TEAEs was reported by 16 of 24 (66.7%) subjects who received IMB-1018972, and a total of 3 TEAEs was reported by 2 of 8 (25%) subjects who received placebo. There were no deaths reported and all TEAEs were transient and resolved without sequelae by follow-up. Subject 131 of the FE arm Group A4 was withdrawn from the study due to a moderate SAE of influenza like illness (unlikely related) in the first period after receiving the single oral dose of 150 mg IMB-1018972 under fasted conditions.

Thirty-seven of 48 TEAEs reported with IMB-1018972 or placebo were of mild severity and 11 TEAEs were of moderate severity. No severe TEAEs were reported. The 11 moderate TEAEs were as follows:
Five moderate TEAEs of flushing (reported term was ‘niacin flush’) were reported by 5 subjects (Subjects 117, 122, 123, 124, and 132). Subjects 117, 122, 123, and 124 reported flushing after a single dose of 400 mg IMB-1018972 under fasted conditions. Subject 132 of the FE arm reported flushing after a single dose of 150 mg IMB-1018972 under fasted conditions. These TEAEs were all considered by the Investigator to be definitely related to the study drug. The niacin flushing events observed in this study were typically short-lasting with generalized cutaneous vasodilation and to varying degrees associated with an intense burning and tingling sensation of the skin, a feeling of warmth, and/or generalized erythema, starting shortly after intake of the drug and lasting about 1 to 2.5 hours.
One moderate TEAE each of restlessness, back pain, and nausea was reported by 1 subject (Subject 122) who also reported a moderate TEAE of flushing. This subject had received a single dose of 400 mg IMB-1018972 under fasted conditions. The TEAEs of back pain and nausea were considered by the Investigator to be possibly related to the study drug, whereas the Investigator considered the TEAE of restlessness to be likely related.
One moderate TEAE each of tonsillitis and post procedural hemorrhage (reported term was ‘post tonsillectomy hemorrhage’) was reported by 1 subject (Subject 129). This subject had received a single dose of 150 mg IMB-1018972 under fed conditions. The TEAEs of tonsillitis and post procedural hemorrhage were considered by the Investigator not to be related to the study drug.
One TEAE of influenza like illness of moderate severity was reported by 1 subject (Subject 131) and was considered by the Investigator to be an SAE and unlikely related to the study drug. This subject had received a single dose of 150 mg IMB-1018972 under fasted conditions and did not receive the planned dose in the fed state..

Of 48 TEAEs, 3 were reported by 2 (25.0%) subjects receiving placebo, 3 were reported by 3 (50.0%) subjects receiving 50 mg IMB-1018972 under fasted conditions, 5 were reported by 3 (50.0%) subjects receiving 150 mg IMB-1018972 under fasted conditions, 16 were reported by 6 (100%) subjects receiving 400 mg IMB-1018972 under fasted conditions, 17 were reported by 4 (66.7%) subjects receiving 150 mg IMB-1018972 under fasted conditions (in the fasted-fed group), and 4 were reported by 1 (20%) subject receiving 150 mg IMB-1018972 under fed conditions (in the fasted-fed group). There was no clear dose dependency of the number and incidence of TEAEs. Neither was there any clear difference between fasted and fed IMB-1018972 administration for the number and incidence of TEAEs.

The most frequently reported TEAEs (ie, reported by ≥15% of the subjects) with IMB-1018972 by system organ class (SOC) were:
Nervous system disorders with 9 TEAEs reported by 7 (29.2%) subjects (4 TEAEs of dizziness, 3 TEAEs of headache, and 1 TEAE each of burning sensation and somnolence).
Vascular disorders with 7 TEAEs reported by 7 (29.2%) subjects (6 TEAEs of flushing and 1 TEAE of peripheral coldness).
General disorders and administration site conditions with 9 TEAEs reported by 5 (20.8%) subjects (3 TEAEs of medical device site pruritus, 2 TEAEs of influenza like illness, and 1 TEAE each of catheter site related reaction, fatigue, feeling hot, and pyrexia).
Gastrointestinal disorders with 9 TEAEs reported by 4 (16.7%) subjects (4 TEAEs of nausea and 1 TEAE each of diarrhea, dry mouth, dysphagia, gingival pain, and vomiting).
Skin and subcutaneous tissue disorders with 4 TEAEs reported by 4 (16.7%) subjects (3 TEAEs of dermatitis contact and 1 TEAE of erythema).

Of 45 TEAEs reported with IMB-1018972, 21 TEAEs reported by 7 of 24 (29.2%) subjects were considered by the Investigator to be related to the study drug. No drug-related TEAEs were reported following 50 mg and 150 mg (fasted only group) IMB-1018972. The most frequently reported drug-related TEAEs (ie, reported by ≥15% of the subjects) with IMB-1018972 by SOC were:
Vascular disorders with 6 TEAEs of flushing reported by 6 (25%) subjects.
Nervous system disorders with 5 TEAEs reported by 4 (16.7%) subjects (3 TEAEs of headache and 2 TEAEs of dizziness).

### TEAEs Reported with Administration of Trimetazidine

A total of 4 TEAEs was reported by 3 of 8 (37.5%) subjects who received trimetazidine. There were no deaths reported and all TEAEs were transient and resolved without sequelae by follow-up. All 4 TEAEs (1 TEAE each of neck pain, abdominal pain, pollakiuria, and headache) reported were of mild severity and considered by the Investigator not to be related to the study drug.

### Overall Tolerability

In the SAD part, treatment with single oral doses of 50 mg, 150 mg, and 400 mg IMB-1018972 under fasted conditions, treatment with single oral doses of 150 mg IMB-1018972 under fed conditions, and treatment with single oral doses of 35 mg trimetazidine were well tolerated by healthy male and female subjects. In the FE arm of the SAD part, dosing under fed conditions appeared to attenuate the number and incidence of TEAEs. During the SAD part, the most common AEs were 6 TEAEs of flushing (reported terms were ‘niacin flush’ and ‘flushing neck’), of which 5 TEAEs were of moderate severity and 1 TEAE was of mild severity. Four subjects reported flushing after a single dose of 400 mg IMB-1018972 under fasted conditions, and 2 subjects of the FE arm reported flushing after a single dose of 150 mg IMB-1018972 under fasted conditions. These TEAEs were all considered by the Investigator to be related to the study drug. No subjects dropped out due to flushing and flushing was not considered a safety issue. There were no clinically important trends in the physical examinations, vital signs, clinical laboratory, or ECG results. Dose escalation beyond 400 mg IMB-1018972 IR did not proceed as planned based on the PK exposure levels of IMB-1028814 and trimetazidine exceeding the target exposure levels in the 400 mg group and the findings of flushing at that dose. The predefined target exposure level was approximately 3 to 4 ‘trimetazidine equivalents’, ie, the ratio of the combined exposure of the active metabolites of IMB1018972 to the single oral doses of 35 mg MR trimetazidine as seen in published literature.

### MAD Part

A total of 35 TEAEs was reported by 14 of 18 (77.8%) subjects who received IMB-1018972, and a total of 17 TEAEs was reported by 5 of 6 (83.3%) subjects who received placebo. All TEAEs were of mild severity and there were no deaths reported. The majority of the TEAEs were transient and resolved without sequelae by follow-up. Three TEAEs were still ongoing at follow-up: vessel puncture site hematoma, medical device site irritation, and paresthesia of the left hand.

Of 35 TEAEs reported by subjects receiving IMB-1018972, 14 were reported by 7 (77.8%) subjects receiving 150 mg IMB-1018972 q12h under fed conditions, and 21 were reported by 7 (77.8%) subjects receiving 50 mg IMB-1018972 q12h under fed conditions. There was no clear dose dependency of the number and incidence of TEAEs.

The most frequently reported TEAEs (ie, reported by ≥25% of the subjects) with IMB-1018972 by SOC were:
Vascular disorders with 7 TEAEs of flushing reported by 6 (33.3%) subjects.
General disorders and administration site conditions with 7 TEAEs reported by 5 (27.8%) subjects (1 TEAE each of catheter site hematoma, chest discomfort, fatigue, feeling hot, medical device site erythema, medical device site irritation, and vessel puncture site hematoma).
Musculoskeletal and connective tissue disorders with 7 TEAEs reported by 5 (27.8%) subjects (2 TEAEs of myalgia and 1 TEAE each of muscle twitching, muscular weakness, musculoskeletal pain, neck pain, and pain in extremity).

Of 35 TEAEs reported with IMB-1018972, 7 TEAEs reported by 6 of 18 (33.3%) subjects were considered by the Investigator to be related to the study drug and 28 TEAEs reported by 11 of 18 (61. 1%) subjects were considered by the Investigator not to be related to the study drug. All 7 reported drug-related TEAEs were events of flushing and all of these were reported following the highest multiple dose of 150 mg IMB-1018972 q12h under fed conditions.

### Overall Tolerability

Fourteen-day treatment with oral q12h doses of 50 mg and 150 mg IMB-1018972 under fed conditions was well tolerated by healthy male and female subjects. Incidental mild TEAEs of flushing occurred in 6 subjects who had received 150 mg IMB-1018972 q12h.Five of these 6 subjects reported only a single TEAE of flushing during the 14 days dosing period. One subject reported flushing twice, on Day 2 and on Day 14. The severity of flushing was less in the 150 mg IR MAD group relative to that in the 400 mg IR SAD group. No TEAEs of flushing were reported following administration of 50 mg IMB-1018972 q12h.No subjects dropped out and no modification of the dose was needed due to the TEAEs of flushing.

### Deaths, Other Serious Adverse Events, and Other Significant Adverse Events

One subject was withdrawn during the study.

Subject 131 was a 25-year old white male with a BMI of 21.9 kg/m2. The subject participated in the FE arm Group A4 and was planned to receive 150 mg IMB-1018972 under fasted conditions in the first treatment period and 150 mg IMB-1018972 under fed conditions in the second treatment period. Initially, he reported no relevant medical history and received no concomitant medication at baseline. The subject received a single dose of 150 mg IMB-1018972 under fasted conditions on Day 1 of the first period. Within half an hour after dosing, the subject reported mild short-lasting TEAEs of dizziness, feeling hot, flushing, nausea, and dysphagia, which were all considered by the Investigator to be likely related. He recovered swiftly and completely, and safety assessments including clinical laboratory results showed no abnormalities throughout the in-house period. The subject left the clinic on Day 3 as planned. On Day 5, the subject was assessed by a healthcare provider for the event of flu like symptoms and spontaneous generalized myalgia. On Day 6, the subject was also assessed for the event of anuria despite ample fluid intake. On Day 7, the subject was referred to a hospital where he was immediately hydrated intravenously. Diuresis did not resume immediately and consequently he was admitted to the hospital. The subject’s body temperature on admission was 38° C. Hydration was continued and during the evening and night diuresis resumed. The subject’s clinical condition improved rapidly, and the subject was discharged on Day 8. Further medical history elucidated dengue fever (December 2018) and viral infection of unknown origin (January 2019) in the months prior to the clinical study and were added to the subject’s medical history (this medical history has not been added to the database). A nonspecific diagnosis was established in the hospital. The hospital summarized the event as anuria with normal renal functions, no abnormalities in urinalysis, and resumption of diuresis during admission. The Investigator reported normal renal function and no rhabdomyolysis. The events of flu like symptoms, myalgia, and anuria together were recorded as an SAE of ‘influenza like illness’ starting on Day 5 and ending approximately 8 days later, on Day 13. This SAE was of moderate severity and considered by the Investigator to be unlikely related to the study drug. The subject did not receive the planned dose of 150 mg IMB-1018972 under fed conditions in the second treatment period. The subject returned on Day 15 for a follow-up with safety assessments conducted as planned. The subject received 37.5 mg tramadol twice daily on Days 6 and 7 and 1000 mg paracetamol twice daily on Days 7 and 8 because of the flu like symptoms. The subject also reported mild TEAEs of back pain from Day 1 to Day 2 (not related), medical device site pruritus on Day 2 (not related), erythema on Day 2 (unlikely related), and burning sensation from Day 2 to Day 5 (unlikely related).

The SAE of ‘influenza like illness’ that led to the withdrawal of Subject 131 from the study was considered by the Investigator to be unlikely related to the study drug due to its weak time-relationship with study drug administration. The Investigator considers this SAE may have been caused by an infection.

### Concomitant Treatment

### SAD Part (and Integrated FE Arm)

Eighteen subjects in the SAD part (with integrated FE arm) received or took concomitant medication. Fifteen female subjects used contraception during the study. In addition, 4 subjects received concomitant medication as follows:
One subject (Subject 103; 50 mg IMB-1018972 under fasted conditions) received triamcinolone once daily for 2 days because of contact dermatitis on the chest (preferred term: contact dermatitis).
One subject (Subject 116 150 mg IMB-1018972 under fasted conditions) received 500 mg paracetamol once because of dizziness.
One subject (Subject 129; 150 mg IMB-1018972 under fasted conditions [fasted-fed group]) received 1000 mg paracetamol once or twice per day twice because of headache, and once because of muscular cramps of the upper legs (preferred term: muscle spasms).

The same subject also received 5 mg oxycodone 4 times a day for 12 days, 1000 mg paracetamol 4 times a day for 5 days, 80 mg macrogol 4 times a day for 12 days, and 200 mg celecoxib once daily for 5 days because of tonsillitis.
One subject (Subject 131; 150 mg IMB-1018972 under fasted conditions [fasted-fed group]) received 37.5 mg tramadol twice daily for 2 days and 1000 mg paracetamol twice daily for 2 days because of flu like disease (preferred term: influenza like illness).

These medications were not considered to have influenced the outcome of the study

### MAD Part

Seven subjects in the MAD part received or took concomitant medication. Six female subjects used contraception during the study. In addition, 1 subject (Subject 221; placebo q12h under fed conditions) received concomitant 500 mg paracetamol once because of a sore throat (preferred term: oropharyngeal pain).

These medications were not considered to have influenced the outcome of the study.

### Safety Conclusions

Overall, single oral IMB-1018972 doses and multiple oral IMB-1018972 doses of an IR formulation, were generally well tolerated by healthy male and female subjects. There were no findings of clinical relevance with respect to clinical laboratory, vital signs, 12-lead ECG, continuous cardiac monitoring (telemetry), or physical examination. Of note, there were no findings of hemodynamic changes, nor changes in the QTc-interval, after administration of IMB-1018972 as the IR formulations.
During the SAD part, the most common AEs were 6 TEAEs of flushing (reported terms were ‘niacin flush’ and ‘flushingneck’), of which 5 TEAEs were of moderate severity and 1 TEAE was of mild severity. Four subjects reported flushing after a single dose of 400 mg IMB-1018972 under fasted conditions, and 2 subjects of the FE arm reported flushing after a single dose of 150 mg IMB-1018972 under fasted conditions. These TEAEs were all considered by the Investigator to be related to the study drug. No subjects dropped out due to flushing and flushing was not considered a safety issue. Dose escalation beyond 400 mg IMB-1018972 IR did not proceed as
planned based on the PK exposure levels of IMB-1028814 and trimetazidine exceeding the target exposure levels in the 400 mg group and the findings of flushing at that dose. The predefined target exposure level was approximately 3 to 4 ‘trimetazidine equivalents’, ie, the ratio of the combined exposure of the active metabolites of IMB-1018972 to the single oral doses of 35 mg MR trimetazidine as seen in published literature.
There were no deaths reported during the study. Most TEAEs were of mild severity and no severe TEAEs were reported during the study. Overall, 12 of a total of 181 TEAEs were of moderate severity.
Two subjects were withdrawn from the study: 1 subject due to a moderate SAE of influenza like illness (unlikely related) and 1 due to a moderate TEAE of ALT increased (possibly related).
Overall, there was no clear dose dependency of the number and incidence of TEAEs.
Dosing under fed conditions appeared to attenuate the number and incidence of TEAEs in the FE arm of the SAD part.

### Discussion and Overall Conclusions

This was a double-blind, randomized, placebo-controlled study, consisting of a SAD part with integrated FE arm, and a MAD part to assess the safety, tolerability, and PK of ascending single and multiple oral doses of IMB-1018972 (IR formulation in the SAD and MAD parts), and single oral doses of a MR formulation of trimetazidine. The study started with the SAD part

### Safety Discussion

Overall, single oral IMB-1018972 doses and multiple oral IMB-1018972 doses of an IR formulation were generally well tolerated by healthy male and female subjects. There were no findings of clinical relevance with respect to clinical laboratory, vital signs, 12-lead ECG, continuous cardiac monitoring (telemetry), or physical examination. Of note, there were no findings of hemodynamic changes, nor changes in the QTc-interval, after administration of IMB-1018972 as the IR formulations.

Nicotinic acid (niacin) is an immediate hydrolysis product of IMB-1018972 and constitutes approximately 30% of the molecular mass of IMB-1018972. In this study, TEAEs of flushing, of which the characteristics were consistent with the flushing seen with the administration of niacin, were reported. All events were transient and resolved without intervention. No subjects dropped out and no modification of the dose was needed due to the TEAEs of flushing.

In the SAD (IR) part of the study, the most common AEs were 6 TEAEs of flushing (reported terms were ‘niacin flush’ and ‘flushingneck’), of which 5 TEAEs were of moderate severity and 1 TEAE was of mild severity. Four subjects reported flushing after a single dose of 400 mg IMB-1018972 under fasted conditions, and 2 subjects of the FE arm reported flushing after a single dose of 150 mg IMB-1018972 under fasted conditions.

In the 14-day multiple dose part of the study, subjects received 150 or 50 mg IR IMB-1018972 q12h in the fed state. Six subjects in the 150 mg q12h group reported single instances of flushing that were mild in severity. No TEAEs of flushing were reported following administration of 50 mg IR IMB-1018972 q12h.

One subject was withdrawn from the study. One subject of the FE arm Group A4 was withdrawn from the study due to a moderate SAE of ‘influenza like illness’ following administration of a single oral dose of 150 mg IMB-1018972 under fasted conditions. The SAE of influenza like illness was considered by the Investigator unlikely to be related to the study drug.

The most frequently reported TEAEs during the study were of the SOC vascular disorders (mainly TEAEs of flushing), general disorders and administration site conditions, nervous system disorders, gastrointestinal disorders, and musculoskeletal and connective tissue disorders. The majority of the TEAEs reported during the study were considered by the Investigator not to be related to the study drug.

### Pharmacokinetics

Based on the single-dose and multiple-dose PK results obtained for IMB-1028814 and trimetazidine in this study.

### Safety- Conclusion

Overall, single oral IMB-1018972 doses and multiple oral IMB-1018972 doses of an IR formulation were generally well tolerated by healthy male and female subjects. There were no findings of clinical relevance with respect to clinical laboratory, vital signs, 12-lead ECG, continuous cardiac monitoring (telemetry), or physical examination. Of note, there were no findings of hemodynamic changes, nor changes in the QTc-interval, after administration of IMB-1018972 either as the IR or MR formulations.
During the SAD part, the most common AEs were 6 TEAEs of flushing (reported terms were ‘niacin flush’ and ‘flushingneck’), of which 5 TEAEs were of moderate severity and 1 TEAE was of mild severity. Four subjects reported flushing after a single dose of 400 mg IMB-1018972 under fasted conditions, and 2 subjects of the FE arm reported flushing after a single dose of 150 mg IMB-1018972 under fasted conditions. These TEAEs were all considered by the Investigator to be related to the study drug. No subjects dropped out due to flushing and flushing was not considered a safety issue. Dose escalation beyond 400 mg IMB-1018972 IR did not proceed as planned based on the PK exposure levels of IMB-1028814 and trimetazidine exceeding the target exposure levels in the 400 mg group and the findings of flushing at that dose. The predefined target exposure level was approximately 3 to 4 ‘trimetazidine equivalents’, ie, the ratio of the combined exposure of the active metabolites of IMB-1018972 to the single oral doses of 35 mg MR trimetazidine as seen in published literature.
There were no deaths reported during the study. Most TEAEs were of mild severity and no severe TEAEs were reported during the study. Overall, 12 of a total of 181 TEAEs were of moderate severity.
One subject was withdrawn from the study: 1 subject due to a moderate SAE of influenza like illness (unlikely related) and 1 due to a moderate TEAE of ALT increased (possibly related).
Overall, there was no clear dose dependency of the number and incidence of TEAEs.
Dosing under fed conditions appeared to attenuate the number and incidence of TEAEs in the FE arm of the SAD part.

### Pharmacokinetics - Conclusions

IMB-1018972 could be measured in only few plasma samples taken during this study.
When combining the single and multiple IMB-1018972 dose results under fasted and fed conditions, including those of the MR formulations, the initial hydrolysis of IMB-1018972 to IMB-1028814 and subsequent systemic bioavailability of IMB-1028814 was relatively rapid with median tmax ranging between 0.5 hours and 5 hours postdose for IMB-1028814, and between 1.5 hours and 8 hours postdose for trimetazidine. Median tmax did not increase with increasing IMB-1018972 dose.
The predefined stopping criterion for IMB-1028814 plasma exposure of 417,733 and 652,849 ng.h/mL for males and females, respectively, was not reached by any of the subjects during the SAD part or MAD part.
Following single oral IMB-1018972 doses in the range of 50 to 400 mg under fasted conditions, systemic exposure to IMB-1028814 and trimetazidine was dose proportional for Cmax, AUC₀₋ₜ, and AUC_{0-inf}.
No evidence for an effect of food was observed on the IMB-1028814 exposure parameters AUC₀₋ₜ and AUC_{0-inf} following administration of a single dose of 150 mg IMB-1018972. However, Cₘₐₓ was approximately 36% lower following administration of a single dose of 150 mg IMB-1018972 under fed conditions relative to administration under fasted conditions.
No evidence for an effect of food was observed on the trimetazidine exposure parameters Cₘₐₓ, AUC₀₋ₜ, and AUC_{0-inf} following administration of a single dose of 150 mg IMB-1018972.
Within 48 hours following administration of a single oral dose of IMB-1018972 over the range of 50 mg to 400 mg, on average between 3.99% and 5.74% of the dose was excreted in urine as IMB-1028814, and on average between 23.11% and 32.55% of the dose was excreted in urine as trimetazidine.
Within 48 hours following administration of a single oral dose of 35 mg trimetazidine, on average 54.47% of the dose was excreted in urine as trimetazidine.
Following 14 days of twice daily dosing with 150 mg and 50 mg IMB-10818972 under fed conditions, no relevant accumulation was observed of IMB-1028814 (R_{ac} of 1.18 and 1.10 for 150 mg and 50 mg, respectively), and accumulation of trimetazidine was modest (R_{ac} of 1.63 and 1.89 for 150 mg and 50 mg, respectively).

### Overall

In view of the positive risk/benefit profile and the observed PK characteristics of the IMB-1018972 metabolites IMB-1028814 and trimetazidine in this single-dose and multiple-dose FIH study, further clinical development of IMB-1018972 is warranted.

### INCORPORATION BY REFERENCE

References and citations to other documents, such as patents, patent applications, patent publications, journals, books, papers, web contents, have been made throughout this disclosure. All such documents are hereby incorporated herein by reference in their entirety for all purposes.

### EQUIVALENTS

Various modifications of the invention and many further embodiments thereof, in addition to those shown and described herein, will become apparent to those skilled in the art from the full contents of this document, including references to the scientific and patent literature cited herein. The subject matter herein contains important information, exemplification, and guidance that can be adapted to the practice of this invention in its various embodiments and equivalents thereof.

What is claimed is:

1. A method of treating a cardiovascular condition in a subject, the method comprising providing to a subject having, or at risk of developing, a cardiovascular condition at least one dose per day of a composition comprising a compound of formula (X): 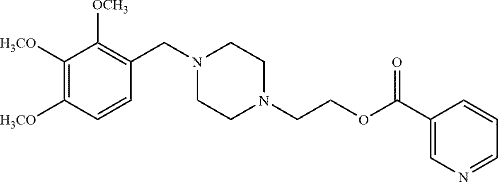 [SMILES: COc1ccc(CN2CCN(CCOC(=O)c3cccnc3)CC2)c(OC)c1OC]
.

2. The method of claim 1, wherein the at least one dose is provided orally.

3. The method of claim 1, wherein the composition is provided in two doses per day.

4. The method of claim 1, wherein the at least one dose comprises from about 25 mg to about 1000 mg of the compound of formula (X).

5. The method of claim 4, wherein the at least one dose comprises from about 100 mg to about 400 mg of the compound of formula (X).

6. The method of claim 5, wherein the at least one dose comprises about 200 mg of the compound of formula (X).

7. The method of claim 1, wherein the at least one dose is provided daily for at least two weeks.

8. The method of claim 1, wherein the cardiovascular condition is selected from the group consisting of aneurysm, angina, atherosclerosis, atherosclerosis, atherosclerosis, atherosclerosis, cardiomyopathy, cerebral vascular disease, congenital heart disease. coronary artery disease, coronary heart disease, diabetic cardiomyopathy, heart attack, heart disease, heart failure, hypertension, ischemic heart disease, pericardial disease, peripheral arterial disease, rheumatic heart disease, stroke, transient ischemic attacks, and valvular heart disease.

9. The method of claim 8, wherein the cardiovascular condition is angina.

10. The method of claim 9, wherein the angina is refractory to other medical interventions.

11. A method of improving cardiac mitochondrial function in a subject, the method comprising providing to a subject at least one dose per day of a composition comprising a compound of formula (X): 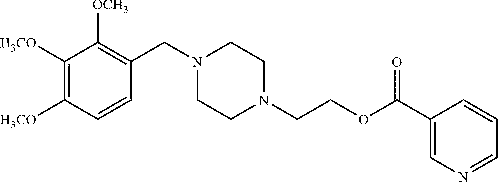 [SMILES: COc1ccc(CN2CCN(CCOC(=O)c3cccnc3)CC2)c(OC)c1OC]
.

12. The method of claim 11, wherein the at least one dose is provided orally.

13. The method of claim 11, wherein the composition is provided in two doses per day.

14. The method of claim 11, wherein the at least one dose comprises from about 25 mg to about 1000 mg of the compound of formula (X).

15. The method of claim 14, wherein the at least one dose comprises from about 100 mg to about 400 mg of the compound of formula (X).

16. The method of claim 15, wherein the at least one dose comprises about 200 mg of the compound of formula (X).

17. The method of claim 11, wherein the at least one dose is provided daily for at least two weeks.

18. The method of claim 11, wherein the subject has, or is at risk developing, a condition selected from the group consisting of aneurysm, angina, atherosclerosis, atherosclerosis, atherosclerosis, atherosclerosis, cardiomyopathy, cerebral vascular disease, congenital heart disease. coronary artery disease, coronary heart disease, diabetic cardiomyopathy, heart attack, heart disease, heart failure, hypertension, ischemic heart disease, pericardial disease, peripheral arterial disease, rheumatic heart disease, stroke, transient ischemic attacks, and valvular heart disease.

19. The method of claim 18, wherein the condition is angina.

20. The method of claim 19, wherein the angina is refractory to other medical interventions.
